(19) United States
(12) Patent Application Publication
(10) Pub. No.: US 2026/0182835 A1
(43) Pub. Date: Jul. 2, 2026

(54) SYSTEM AND METHOD FOR AMBIENT-LIGHT-CORRECTED COMPUTATIONAL PUPILLOMETRY

(71) Applicant: Solvemed Group Sp. z o.o., Poznan (PL)

(72) Inventors: Radoslaw Tomasz CHRAPKIEWICZ, Sunnyvale, CA (US); Hugo CHROST, Campton, NH (US); Marek DZIUBINSKI, Warsaw (PL); Michal WLODARSKI, Bydgoszcz (PL)

(21) Appl. No.: 19/435,611

(22) Filed: Dec. 29, 2025

Related U.S. Application Data
(63) Continuation-in-part of application No. PCT/PL2025/000015, filed on Jun. 11, 2025.
(60) Provisional application No. 63/740,657, filed on Dec. 31, 2024.

(51) Int. Cl. A61B 3/11; A61B 3/00; G16H 10/60

(52) U.S. Cl. CPC A61B 3/112; A61B 3/0016; G16H 10/60; A61B 2560/0223

(57) ABSTRACT

A computational pupillometry system comprises an imaging device configured to capture video frames of a subject's eye and processors executing instructions to perform advanced pupillary assessment. The system employs multi-frame integration techniques, including super-resolution algorithms that utilize sub-pixel shifts between frames, temporal averaging for noise reduction, and parallax-based artifact mitigation to enhance measurement accuracy. Artificial intelligence models, including temporal neural networks, analyze the enhanced pupillary data to determine pupillary parameters and calculate a light-invariant Pupil Reactivity (PuRe) score. The system processes ambient lighting conditions through computational models that analyze video frames before and after controlled stimulation, enabling consistent scoring across varying environmental conditions. Quality assurance mechanisms provide pre-recording and post-recording validation with real-time feedback. The system integrates with electronic medical records through standardized healthcare protocols and supports synchronized, multi-device deployment across healthcare networks.

### CROSS-REFERENCE TO RELATED APPLICATIONS

This application claims the benefit of U.S. Provisional Application No. 63/740,657 filed Dec. 31, 2024, and a Continuation-in-Part of International Application No PCT/PL2025/000015, filed on Jun. 11, 2025. The entire content of each of the applications referenced in this paragraph is hereby incorporated by reference in its entirety herein for all purposes and made a part of this specification.

### FIELD

The present disclosure relates to pupillometry systems and methods for measuring pupillary responses and generating outputs that incorporate ambient illumination context.

### BACKGROUND

Pupillometry encompasses the quantitative measurement and analysis of pupil size and its dynamic responses, including the pupillary light reflex (PLR), and is used as part of neurological examinations in clinical settings. Quantitative pupillometry can provide objective measurements that can be performed on conscious and unconscious patients and can include parameters such as initial pupil diameter, constricted pupil diameter, latency of constriction onset, and constriction and dilation dynamics.

### SUMMARY

The present disclosure relates to computational pupillometry systems and methods. In embodiments, an imaging device captures a sequence of video frames of a subject's eye before, during, and after a controlled light stimulation protocol. A hardware processor executes instructions to measure pupillary response parameters from the video sequence and to generate outputs related to pupillary function.

In embodiments, the system measures or estimates ambient illumination conditions and uses such illumination context when generating a normalized pupil reactivity score. Ambient illumination can be measured with a light sensor (e.g., a photodiode, a dedicated ambient light sensor, or other illumination sensor) and/or can be estimated from the captured video frames. In embodiments, statistical intensity features are extracted from a region of interest in one or more video frames and processed through a computational model or a machine learning model to estimate ambient illumination. The estimation can be calibrated using training data spanning multiple illumination levels, and can include compensating for illumination contributed by the controlled light stimulation. In embodiments, estimating ambient illumination comprises using intensity statistics from pre-stimulus frames and/or from frames spanning a transition into or out of a controlled illumination interval (e.g., a flash-onset transition), optionally normalized or conditioned on one or more camera settings (e.g., exposure time, gain, ISO, white balance, and/or aperture). In embodiments, baseline pupil diameter (INIT) is additionally used as a proxy or auxiliary input for estimating ambient illumination and/or for selecting a light-dependent scoring parameterization, for example when a dedicated light sensor is absent, saturated, or unreliable.

In embodiments, a normalized pupil reactivity score is calculated using a function or a machine learning model that incorporates pupillary response parameters and the measured or estimated ambient illumination conditions. The normalized score can be scaled to a defined range and compared to thresholds to support clinical interpretation. In embodiments, the system classifies a pupil as unreactive when a measured constriction amplitude (CAMP) or change in diameter (Δ) is below a threshold, where the threshold can depend on ambient illumination and baseline pupil diameter (INIT). In embodiments, the system generates outputs indicative of neurological status, including outputs associated with patient severity assessment and outputs indicative of elevated intracranial pressure or intracranial hypertension risk.

In embodiments, a pupillometry device performs quality assessment of acquired data and provides real-time guidance to a user to improve measurement conditions. In embodiments, the pupillometry device operates in a networked deployment by storing results locally and, when connectivity is available, securely uploading and synchronizing results to a server and/or an electronic medical record system. In embodiments, a second pupillometry device can retrieve synchronized results for a patient from the server to support continuity across care settings. In embodiments, updated parameters or models can be distributed from the server to the pupillometry device (and optionally to the second pupillometry device) with model version tracking. In embodiments, the device uses multi-frame processing to improve measurement robustness and precision.

Other technical features may be readily apparent to one skilled in the art from the following figures, descriptions, and claims.

In some aspects, the techniques described herein relate to a pupillometry system, including: an imaging device configured to capture a sequence of video frames of an eye of a subject at least during a stimulus period when the eye is illuminated by a light stimulus; a light source configured to emit the light stimulus toward the eye; a user interface; and at least one processor and a memory storing instructions that, when executed by the at least one processor, cause the at least one processor to: determine a level of ambient illumination associated with the sequence of video frames using one or more of a sensor signal, at least one video frame in the sequence video frames, and a camera exposure setting; determine one or more pupillary response parameters based on the sequence of video frames; compute, based on the level of ambient illumination and the one or more pupillary response parameters, a normalized pupil reactivity score; and transmit the normalized pupil reactivity score to one or both the user interface and an external system separated from the pupillometry system.

In some aspects, the techniques described herein relate to a computer-implemented method for pupillometry performed by a pupillometry system including an imaging device, a light source, a user interface, and at least one processor, the method including, by the at least one processor: capturing, using the imaging device, a sequence of video frames of an eye of a subject at least during a stimulus period when the eye is illuminated by a light stimulus; emitting, using the light source, the light stimulus during capture of the sequence of video frames; determining a level of ambient illumination associated with the sequence of video frames using one or more of a sensor signal, at least one video frame in the sequence video frames, and a camera exposure setting; determining one or more pupillary response parameters from the sequence of video frames; computing, a normalized pupil reactivity score based on the level of ambient illumination and the one or more pupillary response parameters; and transmitting the normalized pupil reactivity score to one or both the user interface and an external system separated from the pupillometry system.

In some aspects, the techniques described herein relate to a networked pupillometry system, including: a server system; and a plurality of pupillometry devices communicatively coupled to the server system, the plurality of pupillometry devices including at least a first pupillometry device and a second pupillometry device, wherein the first pupillometry device includes: an imaging device configured to capture a sequence of video frames of an eye of a subject at least during a light stimulus period; a light source configured to emit a light stimulus toward the eye; and at least one processor and a memory storing instructions that, when executed by the at least one processor, cause the at least one processor to: generate, using the sequence of video frames, a first pupillometry measurement record including one or more pupillary response parameters for the eye of the subject; and transmit the first pupillometry measurement record to the server system, in association with a first patient identifier identifying the subject; wherein the server system includes at least one server processor and a server memory storing instructions that, when executed, cause the server system to: receive a plurality of pupillometry measurement records from the plurality of pupillometry devices; store the plurality of pupillometry measurement records each in association with a patient identifier; and provide, to the second pupillometry device, a synchronized patient record view including at least the first pupillometry measurement record generated by the first pupillometry device for the patient identifier.

In some aspects, the techniques described herein relate to a computer-implemented method, including by a processor of a server system of a networked pupillometry system. receiving, from a plurality of pupillometry devices, measurement pupillometry records including a first measurement pupillometry record, in association with a first patient identifier, from a first pupillometry device of the plurality of pupillometry devices, wherein the first patient identifier identifies a subject; store at least the first pupillometry measurement record and the first patient identifier; and provide, to a second pupillometry device of the plurality of pupillometry devices, a synchronized patient record view including the first pupillometry measurement record generated by the first pupillometry device; wherein the first pupillometry includes: an imaging device configured to capture a sequence of video frames of an eye of the subject at least during a light stimulus period; a light source configured to emit the light stimulus toward the eye; and at least one processor and a memory storing instructions that, when executed by the at least one processor, cause the at least one processor to: generate, using the sequence of video frames, a pupillometry measurement record including one or more pupillary response parameters for the eye of the subject; and transmit the pupillometry measurement record to the server system, in association with the first patient identifier.

In some aspects, the techniques described herein relate to a non-transitory computer-readable medium storing instructions that, when executed, by one or more processors, cause the one or more processors to perform operations including: receiving, from a plurality of pupillometry devices, measurement pupillometry records including a first measurement pupillometry record, in association with a first patient identifier, from a first pupillometry device of the plurality of pupillometry devices, wherein the first patient identifier identifies a subject; store at least the first pupillometry measurement record and the first patient identifier; and provide, to a second pupillometry device of the plurality of pupillometry devices, a synchronized patient record view including the first pupillometry measurement record generated by the first pupillometry device; wherein the first pupillometry includes: an imaging device configured to capture a sequence of video frames of an eye of the subject at least during a light stimulus period; a light source configured to emit the light stimulus toward the eye; and at least one processor and a memory storing instructions that, when executed by the at least one processor, cause the at least one processor to: generate, using the sequence of video frames, a pupillometry measurement record including one or more pupillary response parameters for the eye of the subject; and transmit the pupillometry measurement record to the one or more processors, in association with the first patient identifier.

In some aspects, the techniques described herein relate to a networked pupillometry system, including: a server system; and a plurality of pupillometry devices communicatively coupled to the server system, the plurality of pupillometry devices including at least a first pupillometry device having a first device configuration identifier and a second pupillometry device having a second device configuration identifier different from the first device configuration identifier, wherein each pupillometry device includes: an imaging device configured to capture a sequence of video frames of an eye of a subject at least during a light stimulus period; a light source configured to emit a light stimulus toward the eye; and at least one processor and a memory storing instructions that, when executed by the at least one processor, cause the at least one processor to: determine the device configuration identifier for the pupillometry device; obtain, from the server system, a device-specific calibration parameter set associated with the device configuration identifier; estimate, using at least one video frame in the sequence and one or more camera settings and in accordance with the device-specific calibration parameter set, an ambient illumination level associated with the sequence; determine one or more pupillary response parameters for the eye of the subject from the sequence; compute a normalized pupil reactivity score based on the ambient illumination level and the one or more pupillary response parameters; generate a pupillometry measurement record including at least the normalized pupil reactivity score, the one or more pupillary response parameters, the device configuration identifier, and a version identifier for the device-specific calibration parameter set; and transmit the pupillometry measurement record to the server system in association with a patient identifier identifying the subject, wherein the server system includes at least one server processor and a server memory storing instructions that, when executed, cause the server system to: store a plurality of device-specific calibration parameter sets each in association with a respective device configuration identifier, the plurality of device-specific calibration parameter sets including at least (i) a first device-specific calibration parameter set associated with the first device configuration identifier and (ii) a second device-specific calibration parameter set associated with the second device configuration identifier; receive a plurality of pupillometry measurement records from the plurality of pupillometry devices; store the plurality of pupillometry measurement records each in association with a patient identifier; and provide, to the second pupillometry device, a synchronized patient record view including at least a pupillometry measurement record associated with the patient identifier and generated by the first pupillometry device.

In some aspects, the techniques described herein relate to a computer-implemented method performed by a processor of a server system of a networked pupillometry system, the method including: storing, in the server system, a plurality of device-specific calibration parameter sets each in association with a respective device configuration identifier; receiving, from a first pupillometry device of a plurality of pupillometry devices, a request including a first device configuration identifier; in response to the request, selecting a first device-specific calibration parameter set associated with the first device configuration identifier, and providing the first device-specific calibration parameter set to the first pupillometry device; receiving, from the first pupillometry device and in association with a patient identifier identifying a subject, a first pupillometry measurement record including: (i) a normalized pupil reactivity score computed based on an ambient illumination level estimated in accordance with the first device-specific calibration parameter set and (ii) (ii) one or more pupillary response parameters, and further including a version identifier for the first device-specific calibration parameter set; storing the first pupillometry measurement record in association with the patient identifier; and providing, to a second pupillometry device of the plurality of pupillometry devices, a synchronized patient record view including the first pupillometry measurement record.

### BRIEF DESCRIPTION OF THE DRAWINGS

The preceding and other objects, features, and advantages will be apparent from the following more particular description of the embodiments, as illustrated in the accompanying drawings. Like reference, characters refer to the same parts throughout the different views. The drawings are not necessarily to scale, emphasis instead being placed upon illustrating the embodiments' principles. Unless otherwise stated, arrows and/or connecting lines in the drawings generally indicate an exemplary flow of data, control, and/or stored records between blocks, and such arrows/lines are non-limiting.

FIG. 1A is a block diagram of a computational pupillometry device or system in accordance with embodiments of the present disclosure.

FIG. 1B is a block diagram illustrating operations of selected blocks in the processing unit of the computational pupillometry system shown in FIG. 1A depicting input data, process, and output data for each block.

FIG. 2 is a diagram of a light-invariant pupil reactivity score calculation system in accordance with embodiments of the present disclosure.

FIG. 3 is a diagram of a multi-frame integration architecture system in accordance with embodiments of the present disclosure.

FIG. 4 is a diagram of a comprehensive quality control system in accordance with embodiments of the present disclosure.

FIG. 5 is a diagram of a multi-device synchronization system in accordance with embodiments of the present disclosure.

FIG. 6 is a diagram of a multi-parameter confounder analysis and correction system in accordance with embodiments of the present disclosure.

FIG. 7 is a diagram of a saturation function mathematical framework for light-invariant pupil reactivity score calculation in accordance with embodiments of the present disclosure.

FIG. 8 is a diagram of a clinical validation system in accordance with embodiments of the present disclosure.

FIG. 9 is a diagram of a smartphone hardware platform architecture in accordance with embodiments of the present disclosure.

FIG. 10 is a diagram of a clinical user interface and workflow integration system in accordance with embodiments of the present disclosure.

FIG. 11 is a diagram of a method related to normalizing pupillometry measurements across varying environmental lighting conditions in accordance with embodiments of the present disclosure.

FIG. 12 is a diagram of a method related to enhancing pupillometry measurement precision through multi-frame processing in accordance with embodiments of the present disclosure.

FIG. 13 is a diagram of a method related to ensuring pupillometry measurement quality and reliability in accordance with embodiments of the present disclosure.

FIG. 14 is a diagram of a method related to synchronizing pupillometry measurement data across multiple mobile devices in accordance with embodiments of the present disclosure.

FIG. 15 is a diagram of a method related to correcting pupillometry measurements for confounding factors in accordance with embodiments of the present disclosure.

FIG. 16 is a diagram illustrating an example handheld pupillometry acquisition setup, including optional ambient illumination sensing hardware and physical alignment aids in accordance with embodiments of the present disclosure.

FIG. 17 is a diagram illustrating estimation of ambient illumination using one or more of sensor-based measurements and image-based computational estimation, including calibration and machine learning-based prediction, in accordance with embodiments of the present disclosure.

FIG. 18 is a flow diagram illustrating an end-to-end sequence of operation for acquiring a pupillometry video sequence, performing quality gating, estimating ambient illumination, computing a normalized pupil reactivity score, and outputting and synchronizing results in accordance with embodiments of the present disclosure.

FIGS. 19A-19D are plots illustrating stability of a normalized pupil reactivity score in a sample neuro-intensive-care-unit patient cohort across ambient illumination conditions compared to one or more raw constriction parameters, in accordance with embodiments of the present disclosure.

FIGS. 20A-20D are plots illustrating diagnostic performance of a normalized pupil reactivity score for intracranial hypertension screening, in accordance with embodiments of the present disclosure.

FIGS. 21A-21C illustrate an example two-tier neuromonitoring method comprising a non-invasive pupillometry device that provides high-frequency screening and an invasive ICP monitor that provides confirmatory measurements in accordance with embodiments of the present disclosure.

FIGS. 22A-22G are plots illustrating pupillograms (FIGS. 22A-22C) and pupillometry parameter differences (FIGS. 22D-22G) across intracranial pressure levels, in accordance with embodiments of the present disclosure.

FIGS. 23A-23D are diagrams illustrating example user interface screens for patient registration, measurement presentation, patient history review, and longitudinal trend visualization, in accordance with embodiments of the present disclosure.

FIGS. 24A-24C are diagrams illustrating example normal and abnormal ranges for pupil reactivity score (FIG. 24A), and example test results (FIG. 24B) for an eye with anisocoria, abnormal pupil shape (FIG. 24C), depicting differential pupillary reactivity between eyes, in accordance with embodiments of the present disclosure.

FIG. 25 is a diagram illustrating synchronization of pupillometry devices with a server and integration with an electronic medical record system, in accordance with embodiments of the present disclosure.

FIG. 26 is a diagram illustrating deployment of pupillometry screening across a spectrum of care settings, in accordance with embodiments of the present disclosure.

FIGS. 27A-27B are diagrams illustrating longitudinal monitoring results across multiple days showing patient's stroke recovery, including exemplary frames and pupillograms, in accordance with embodiments of the present disclosure.

FIG. 28 is a block diagram illustrating a biometric authentication or identification subsystem that uses iris features, pupil dynamics, and eye-movement features to generate an authentication decision, in accordance with embodiments of the present disclosure.

### DETAILED DESCRIPTION

### Introduction and Overview of Embodiments

Pupillometry, the quantitative measurement of pupil size and its dynamic responses to light stimulation, represents a fundamental diagnostic technique in modern medicine. The pupillary light reflex (PLR) serves as a critical non-invasive biomarker for neurological function, autonomic nervous system activity, and various pathological conditions. Traditional approaches to pupillary assessment have evolved from subjective manual evaluations using simple penlights to sophisticated infrared-based measurement systems, each contributing to our understanding of ocular physiology and its clinical applications.

Pupillometry encompasses the quantitative measurement and analysis of pupil size and its dynamic responses, including the pupillary light reflex (PLR), and is used as part of neurological examinations in clinical settings. Quantitative pupillometry can provide objective measurements that can be performed on conscious and unconscious patients and can include parameters such as initial pupil diameter, constricted pupil diameter, latency of constriction onset, and constriction and dilation dynamics.

Interpretation of pupillary responses can be influenced by acquisition conditions, including environmental illumination. Ambient illumination can affect baseline pupil diameter and dynamic response characteristics and can complicate comparison of measurements captured across different clinical environments and workflows. Accordingly, there is a need for pupillometry systems that measure or estimate ambient illumination and incorporate such context when generating outputs related to pupillary function.

The historical foundation of pupillometry rests on the recognition that pupil diameter and reactivity provide valuable insights into the integrity of neural pathways connecting the eye to the brain. Early clinical assessments relied primarily on visual estimation using handheld flashlights, a method that, while widely accessible, suffered from significant inter-observer variability and lacked quantitative precision. The subjective nature of these assessments led to inconsistent documentation and limited reproducibility, particularly in critical care environments where accurate neurological monitoring is paramount.

The advent of dedicated hardware pupillometers marked a significant advancement in the field of ophthalmology. These specialized devices typically employ infrared illumination and sophisticated optical systems to capture high-contrast images of the pupil against the iris background. Telecentric lens systems enable precise geometric measurements, while controlled lighting conditions minimize environmental interference. Such devices can extract multiple quantitative parameters, including baseline pupil diameter, constricted diameter, constriction and dilation velocities, latency of response, and various temporal characteristics that collectively provide a comprehensive assessment of pupillary function.

Contemporary infrared-based pupillometers represent the current standard of care in many clinical settings. These systems utilize near-infrared wavelengths that do not elicit pupillary constriction while providing excellent contrast for pupil detection algorithms. Computer vision techniques automatically segment pupil boundaries and track dynamic changes throughout the light stimulus protocol. Advanced signal processing methods extract clinically relevant metrics and generate standardized reports that support objective clinical decision-making.

Despite these technological advances, current pupillometry systems operate within significant constraints that limit their widespread adoption and clinical utility. The requirement for controlled environmental conditions, including subdued ambient lighting and patient immobilization, restricts their use to specialized settings. The computational limitations of embedded hardware systems constrain the sophistication of algorithms that can be deployed for noise reduction, artifact management, and advanced feature extraction. Additionally, the high cost of specialized hardware and the need for regular maintenance and calibration present barriers to deployment across diverse healthcare environments.

The emergence of powerful mobile computing platforms has created new opportunities for advancing pupillometry technology. Modern smartphones incorporate multi-core electronic processors, graphics processing units, and dedicated neural processing units, all of which are capable of executing complex algorithms in real-time. High-resolution cameras with sophisticated auto-focus systems and computational photography capabilities enable the capture of detailed ocular imagery. These ubiquitous devices offer the potential to democratize access to quantitative pupillary assessment while reducing costs and improving workflow integration.

Parallel developments in artificial intelligence and computer vision have introduced novel approaches to medical image analysis. Deep learning algorithms can learn complex patterns from large datasets, enabling robust feature extraction and measurement accuracy that approaches or exceeds human performance. Temporal neural networks can analyze video sequences to extract dynamic information that traditional single-frame approaches cannot capture. Generative artificial intelligence models can enhance image quality, remove artifacts, and compensate for challenging acquisition conditions.

Multi-frame image processing techniques, originally developed for computational photography and astronomical imaging, provide solutions to challenges that have traditionally been addressed through hardware constraints. Super-resolution algorithms can combine information from multiple slightly displaced images to achieve effective resolution beyond the native sensor capabilities. Temporal averaging and advanced filtering methods can significantly reduce noise while preserving signal integrity. These computational approaches transform apparent disadvantages of handheld operation into advantageous features for enhanced data extraction.

The convergence of these technological developments has laid the foundation for a new paradigm in pupillometry, leveraging computational capabilities to overcome traditional hardware limitations and potentially providing superior performance characteristics.

Current pupillometry methodologies face substantial technical challenges that limit their accuracy, reliability, and clinical applicability across diverse healthcare environments. The fundamental limitation of existing systems lies in their dependence on highly controlled acquisition conditions and simplified data processing approaches that fail to capture the full richness of pupillary dynamics.

Ambient light interference represents a primary challenge for accurate pupillary assessment. Some of the existing systems may attempt to minimize this issue by using infrared illumination and controlled environments; however, this approach restricts their application to specialized settings and overlooks the complex interactions between ambient lighting conditions and pupillary responses. Variations in environmental illumination can significantly alter baseline pupil diameter and dynamic response characteristics, resulting in measurement inconsistencies that compromise the clinical utility of the measurement. The lack of robust ambient light compensation mechanisms in existing systems results in measurements that are highly dependent on environmental conditions rather than purely reflecting physiological status.

Motion artifacts present another significant challenge, particularly in handheld or portable applications. Traditional approaches attempt to minimize motion through mechanical stabilization and controlled patient positioning. Still, this strategy limits accessibility and ease of use in critical care scenarios where rapid assessment is essential. Micro-movements during image acquisition can cause blur, misalignment, and measurement errors, degrading the accuracy of extracted parameters. Current systems lack sophisticated motion compensation algorithms that could transform these apparent disadvantages into opportunities for enhanced measurement precision.

The computational limitations of existing hardware platforms constrain the sophistication of algorithms that can be deployed for signal processing and feature extraction. Simple threshold-based segmentation methods struggle with challenging conditions such as dark irises, small pupils, or the presence of reflections and occlusions. Limited processing power prevents the implementation of advanced temporal analysis techniques that could extract more comprehensive information from pupillary video sequences. The absence of machine learning capabilities restricts the ability of current systems to adapt to varying conditions or learn from accumulated measurement data.

Quality assurance and measurement validation present ongoing challenges in current pupillometry systems. The lack of robust pre-recording and post-recording quality checks can result in unreliable measurements that compromise clinical decision-making. Existing systems often fail to detect and compensate for common artifacts such as blinks, excessive motion, or inadequate lighting conditions. The absence of comprehensive quality metrics and confidence measures limits clinicians' ability to assess the reliability of individual measurements.

Integration with electronic health record systems and clinical workflows remains problematic for many existing pupillometry platforms. Proprietary data formats, limited connectivity options, and complex synchronization requirements impede seamless incorporation into hospital information systems. The lack of standardized measurement protocols and reporting formats complicates cross-platform comparisons and longitudinal patient monitoring across different care settings.

Embodiments of the present disclosure address these fundamental limitations through a comprehensive computational approach that leverages the advanced processing capabilities of modern mobile computing devices to achieve superior pupillometry performance across diverse clinical environments. Rather than attempting to control or minimize environmental challenges, embodiments of the present disclosure computationally process these factors to extract enhanced diagnostic information while maintaining measurement accuracy and reliability.

Some of the methods and systems disclosed below may enable acquisition of pupil parameters independent of certain ambient conditions such as ambient illumination.

The core innovation of embodiments of the present disclosure lies in the strategic application of multi-frame integration techniques that transform apparent disadvantages of mobile device operation into advantageous features for enhanced data extraction. Multi-frame super-resolution algorithms utilize the natural sub-pixel shifts that occur between successive video frames due to physiological eye movements or device micro-movements to reconstruct pupil images with effective resolution beyond the native sensor capabilities. This computational approach enables more precise pupil boundary delineation and diameter measurements than can be achieved with traditional single-frame analysis methods.

Temporal averaging and advanced filtering techniques are employed to reduce measurement noise while preserving clinically relevant signal characteristics to a significant extent. By analyzing multiple aligned frames, embodiments of the present disclosure can achieve signal-to-noise ratios that exceed those obtainable from individual images, even under challenging lighting conditions. Sophisticated denoising algorithms, including deep learning-based approaches, learn complex noise characteristics and spatiotemporal correlations to separate measurement signals from environmental interference effectively.

Parallax-based artifact mitigation represents another key advancement, where micro-movements that occur during handheld operation create differential motion patterns between pupil features and artifacts such as corneal reflections. Temporal neural networks are trained to distinguish consistent pupil structures from transient artifacts based on their motion characteristics across frame sequences. This approach enables effective artifact suppression without requiring specialized hardware or controlled environments.

Advanced artificial intelligence models, including convolutional neural networks, recurrent architectures, and transformer-based systems, are employed to analyze complex spatiotemporal patterns in pupillary video data. These models can learn from extensive datasets that encompass diverse populations, lighting conditions, and physiological states, achieving robust pupil detection and parameter extraction across challenging scenarios. Attention mechanisms enable dynamic focus on relevant image regions while down-weighting artifacts or occlusions.

Embodiments of the present disclosure implement sophisticated ambient light estimation and compensation algorithms that enable accurate measurements across a wide range of environmental conditions. Rather than avoiding ambient light variations, the system analyzes video frames captured before and after light stimulation to extract statistical features that characterize environmental conditions. Machine learning models process these features to accurately predict ambient light levels, enabling computational normalization of pupillary responses independent of environmental factors.

The Pupil Reactivity (PuRe) scoring system represents a fundamental advancement in pupillometry interpretation, providing a standardized metric that maintains consistency across varying lighting conditions while preserving sensitivity to clinically relevant changes. This scoring system employs sophisticated mathematical models that incorporate both pupillary response parameters and ambient light measurements to generate normalized assessments suitable for clinical decision-making and longitudinal monitoring.

Comprehensive quality assurance frameworks are integrated throughout the measurement process, including pre-recording validation of positioning, lighting, and eye detection, as well as post-recording analysis of measurement validity and artifact detection. Real-time feedback guides users to optimal acquisition conditions while automated quality metrics provide confidence measures for individual measurements. These quality control mechanisms ensure reliable results across diverse users and environmental conditions.

Embodiments of the present disclosure incorporate seamless integration capabilities with electronic health record systems through standardized healthcare data protocols and middleware solutions. In embodiments, a pupillometry device stores measurement results locally and, when network connectivity is available, uploads and synchronizes results to a server and/or an electronic health record system so that patient records remain consistent across care settings. In embodiments, a second pupillometry device can download or otherwise retrieve the synchronized results for the same patient. In embodiments, offline operation includes queuing measurements on the pupillometry device and automatically synchronizing when connectivity resumes, thereby maintaining continuous functionality in challenging connectivity environments.

The system architecture supports comprehensive confounder management, incorporating demographic factors such as age and gender, medication histories, and physiological parameters to distinguish between neurologically relevant changes and confounding influences. This multi-dimensional approach enables more accurate clinical interpretation and supports personalized assessment protocols tailored to individual patient characteristics.

A representative implementation of embodiments of the present disclosure was deployed in a neurological intensive care unit where traditional pupillometry assessment methods had proven inadequate for the complex monitoring requirements of critically ill patients. The clinical scenario involved monitoring twelve adult patients with diverse neurological conditions, including hemorrhagic stroke, traumatic brain injury, hydrocephalus, brain tumors, and intracranial hypertension. These patients required frequent pupillary assessments to track their neurological status and guide therapeutic interventions; however, environmental constraints and workflow disruptions limited the use of conventional approaches.

The implemented system demonstrated exceptional measurement accuracy, achieving frame-to-frame pupil diameter precision of ±0.025 mm across all iris colors and lighting conditions encountered in the clinical environment. This accuracy surpassed that of conventional infrared pupillometers while operating in uncontrolled ambient conditions ranging from 4 to 1,200 lux. The system's light-invariant PuRe scoring successfully normalized measurements across this broad range of environmental conditions, with scores showing no significant correlation to ambient light levels while maintaining high sensitivity to neurological changes.

Clinical validation demonstrated a strong correlation between PuRe scores and established neurological assessment metrics, with correlation coefficients exceeding 0.74 with Glasgow Coma Scale scores. The system achieved 84.8% sensitivity and 90.0% specificity for detecting severe neurological impairment, with an area under the receiver operating characteristic curve of 0.940. Prognostic analysis revealed significant differences in PuRe scores between patient outcome groups, with non-survivors showing median scores of 0.00 compared to 2.82 for survivors.

The system's computational approach may enable a reduction in measurement time from 65 seconds, e.g., using existing methods, to 21 seconds per assessment, resulting in approximately 2 hours of nursing time saved per typical ICU patient over a 7-day stay. The seamless integration with electronic health records eliminated manual documentation requirements while providing real-time clinical decision support through automated alerting for concerning pupillary changes.

This clinical implementation demonstrated the practical benefits of computational pupillometry in a demanding healthcare environment, showing how advanced processing techniques can overcome traditional limitations while providing superior clinical utility and workflow integration.

While the foregoing example describes implementation in a neurological intensive care setting, embodiments of the present disclosure are not limited to this specific application. The computational pupillometry systems and methods described herein can be adapted and deployed across a wide range of clinical and research applications where quantitative pupillary assessment provides valuable diagnostic or monitoring information. These applications may include emergency medical services, primary care settings, specialty clinics, rehabilitation facilities, research institutions, and other healthcare environments where portable, accurate, and cost-effective pupillometry would provide clinical benefit. The flexibility of the computational approach enables customization for specific use cases while maintaining the core advantages of enhanced accuracy, environmental adaptability, and seamless workflow integration.

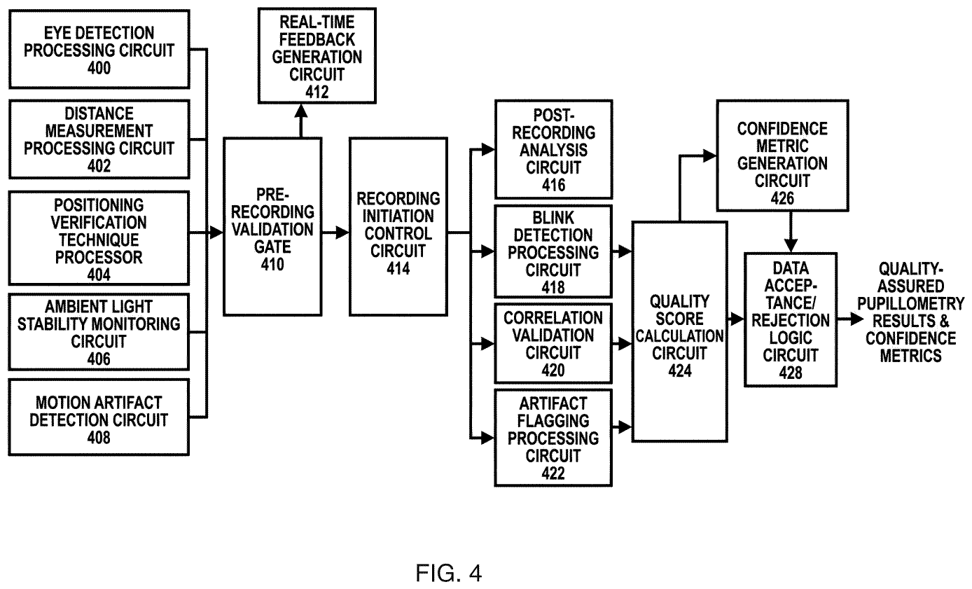

In embodiments, the system performs a quality assessment prior to outputting results (e.g., the pupil reactivity score or other measured or estimated parameters and biomarkers). For example, FIG. 4 illustrates a quality control system that evaluates, among other factors, gaze stability, motion artifacts, blinks, occlusions, focus, distance, and illumination stability. In various implementations, the quality assessment may comprise multiple phases, including an initial validation phase and a final post-recording validation phase that generates the operative quality score. Intermediate enhancement or evaluation steps are optional, and omission of such steps does not preclude generation of the final quality score. In some embodiments, the quality assessment comprises a two-tier process. In a first tier (soft guidance), the system provides real-time guidance to a user (e.g., prompts for repositioning, reducing tilt/rotation, and/or improving alignment) while optionally allowing scoring and output with an associated quality flag. In a second tier (hard gating), the system inhibits outputting the normalized score and/or related classifications, prompts and/or requires recapture, and suppresses storage and/or transmission of compromised results (e.g., to an electronic medical record system) when one or more hard quality criteria are not satisfied (e.g., excessive distance, insufficient focus, severe motion, or occlusion above a threshold). In embodiments, hard gating is applied post-recording and/or in real time during acquisition.

In embodiments, the computational pupillometry platform supports additional, non-limiting embodiments and extensions that can be implemented using the same captured video sequence and/or repeated sequences acquired over time, optionally in combination with other patient data. These embodiments are described to illustrate breadth of application and to support later claiming in a continuation or continuation-in-part.

In embodiments, the processing unit 106 applies one or more models to PuRe measurements and related pupillometry parameters (e.g., INIT, Δ, velocities, latency, and/or morphology features) to output an estimated intracranial pressure (ICP) value and/or a risk probability for intracranial hypertension (e.g., ICP meeting or exceeding a threshold). In embodiments, the model updates the estimate based on repeated measurements over hours or days and outputs early warning scores (e.g., a probability that ICP will cross a threshold within a prediction window), and can generate alerts (e.g., visual, audible, and/or electronic messages).

In embodiments, the system performs multimodal ICP prediction by combining pupillometry features with one or more additional non-invasive ICP surrogates, including optic nerve sheath diameter, ocular ultrasound parameters, transcranial Doppler features, blood pressure, heart rate, and/or other physiological measurements. In embodiments, corneal reflection geometry and/or eye-surface pulsation features derived from flash reflections are used as additional inputs, for example to estimate intraocular pressure and/or to support ICP-risk estimation.

In embodiments, the system receives electroencephalography (EEG) features and combines them with pupillometry features to output a coma state classification, a prognosis score, and/or an awakening probability. Non-limiting EEG feature examples include band-power features (e.g., delta, theta, alpha, and beta power), burst-suppression ratio, and reactivity indices. In embodiments, the model learns patient-specific baselines (e.g., based on an initial recording window) and flags discordant patterns between ocular and EEG features as special risk states.

In embodiments, the system receives imaging-derived features (e.g., from CT, MRI, and/or fMRI) including midline shift, lesion volume, edema metrics, and/or diffusion-related features, and combines them with PuRe and pupillometry parameters to personalize interpretation (e.g., patient-specific thresholds) and/or to estimate risk of neurological deterioration. In embodiments, repeated pupillometry measurements and imaging at multiple time points are used to fit a patient-specific trajectory model.

In embodiments, the system implements a continual learning and deployment loop in which devices collect measurements, quality metrics, ambient illumination, and optional clinical labels or outcomes; models are retrained or recalibrated; and updated models and/or parameters are deployed to devices with model version tracking. In embodiments, a first pupillometry device uploads measurements and associated metadata to a server (or queues such uploads when offline), and the server aggregates datasets to retrain or recalibrate one or more models. In embodiments, the server distributes an updated model and/or updated parameters back to the first pupillometry device and/or to a second pupillometry device, enabling consistent operation across devices. In embodiments, federated learning is used such that local model updates are computed on-device and aggregated without transmitting raw video.

In embodiments, the system may derive additional biomarkers or health metrics from one or more video frames, e.g., the one or more video frames used to estimate or generate the pupil reactivity score. In some cases, a biomarker or health metric that can be measured, estimated, or derived based on one or more video frames, may be referred to as a video-based biomarker. In some cases, multiple video-based biomarkers may be measured, estimated or derived based on the same video frames, or a subset of video frames captured with the same video recording or acquisition period.

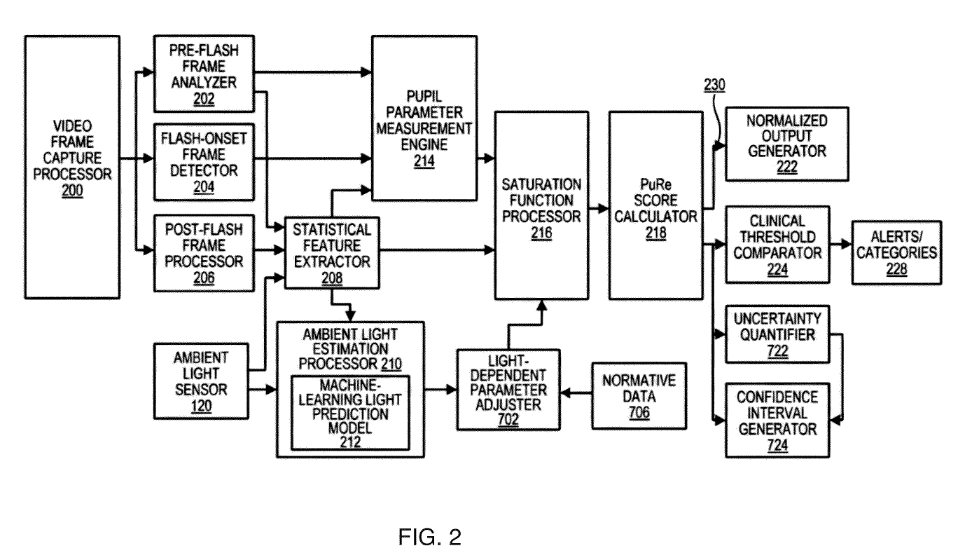

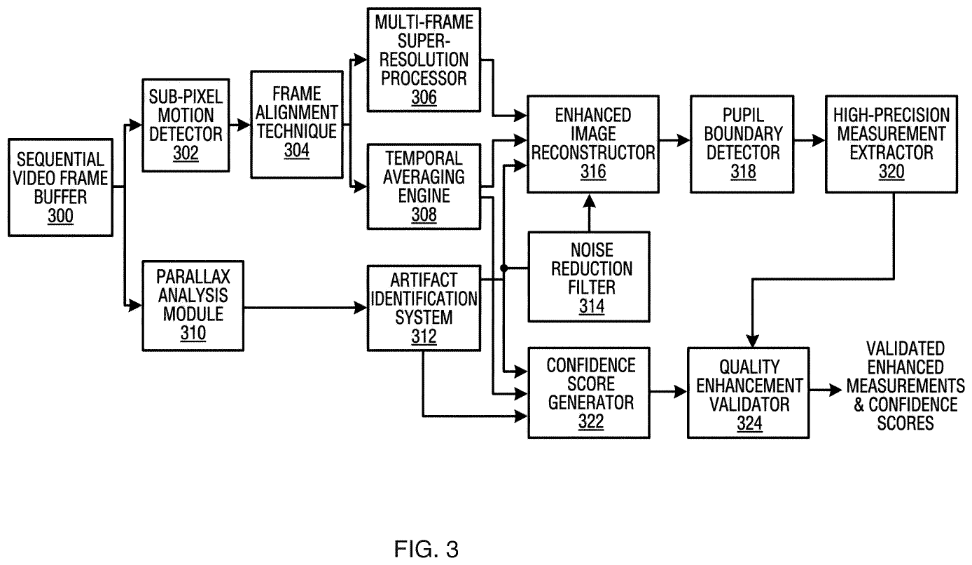

In various embodiments, the additional biomarkers may include but are not limited to: pupil shape/morphology descriptors (e.g., eccentricity, circularity, contour irregularity), anisocoria metrics (static and/or dynamic), pupil roundness scores, eyeball position/orientation and gaze deviation, eyelid metrics (e.g., palpebral fissure height and blink dynamics), spontaneous and/or evoked eye movement patterns (e.g., saccades and nystagmus), and neuro-autonomic oscillation features derived from frequency-domain analysis of a pupillogram. In embodiments, one or more of these biomarkers may be used as additional markers or indicators used to assess neurological health of a subject or patient. For example, one or more of the video-based biomarkers, may be provided as inputs to one or more neurological status models to determine a neurological health or diagnose a neurological disorder. In some cases, one or more of these biomarkers may be derived or determined using a model (neurological status model) and based on at least one input parameter measured or derived using one or more video frames. In some cases, a biomarker (or value of a biomarker) generated by a model or derived from one or more video frames, may be used as an additional clinical decision support indicators (e.g., in addition to pupil reactivity score). As such, some of the methods and systems described above may be used to measure, derive, evaluate, correct/adjust, biomarkers other than pupil reactivity score. For example, the system shown in FIG. 2 may include modules and processes that may extract or derive a video-based biomarker different from pupil reactivity score. As another example, system shown in FIG. 3 may generate a confidence score for a video-based biomarker different from pupil reactivity score.

In embodiments, pupil shape and/or pupil morphology is used as a diagnostic component. In embodiments, the device determines a pupil region using an object detection model that outputs a pupil bounding box (e.g., a YOLO-family detector trained to localize pupil and/or iris regions), and optionally refines the region using a segmentation model to obtain a contour or mask. In embodiments, the device computes one or more shape metrics from the bounding box and/or contour, including aspect ratio, eccentricity, circularity, roundness, contour irregularity, and/or temporal variation of such metrics across frames. In embodiments, the device compares the shape metrics to normative distributions (e.g., stratified by one or more of ambient illumination, baseline pupil diameter, iris characteristics, age, and acquisition geometry) to generate a diagnostic indicator and/or an alert when a deviation exceeds a threshold. Non-limiting examples include alerting on an abnormally non-circular pupil, irregular contour, or abnormal shape dynamics, and/or using such morphology features as additional inputs to neurological status models.

In embodiments, eye movement dynamics are used as an additional diagnostic component derived from the same acquisition using computer vision. In embodiments, the device tracks one or more landmarks over time (e.g., pupil center position, iris features, eyelid landmarks, and/or a glint centroid) using one or more of optical flow, feature tracking, Kalman filtering, and/or a trained neural network (e.g., a detector outputting bounding boxes for pupil/iris regions). In embodiments, the device computes one or more eye movement metrics including gaze deviation, fixation stability, saccade count, saccade amplitude, saccade velocity, smooth pursuit gain, and/or nystagmus signatures (e.g., beat frequency and/or slow-phase velocity). In embodiments, the device compares the eye movement metrics to normative distributions (e.g., stratified by one or more of ambient illumination, acquisition geometry, and/or sedation state) and outputs an additional neurological diagnostic indicator and/or an alert when a deviation exceeds a threshold. In embodiments, such eye movement metrics are additionally used as inputs to one or more neurological status models in combination with PuRe and/or pupillary response parameters.

In embodiments, the same acquisition additionally provides biometric information and can be used as a biometric scan. For example, the device captures an iris image (e.g., iris texture features), pupil dynamics (e.g., a pupillogram and associated response parameters), and eye-movement features (e.g., saccades, fixation patterns, and/or nystagmus), and combines such features to generate a biometric template for subject identification and/or authentication. In embodiments, such identification and/or authentication is used to reduce patient misidentification, to associate a measurement with a correct patient record, and/or to provide liveness checks (e.g., based on dynamic responses and/or eye-movement signatures) beyond a static iris image match. In embodiments, biometric processing is performed on-device and/or using access-controlled comparison against stored templates, and raw video need not be transmitted for such biometric determination.

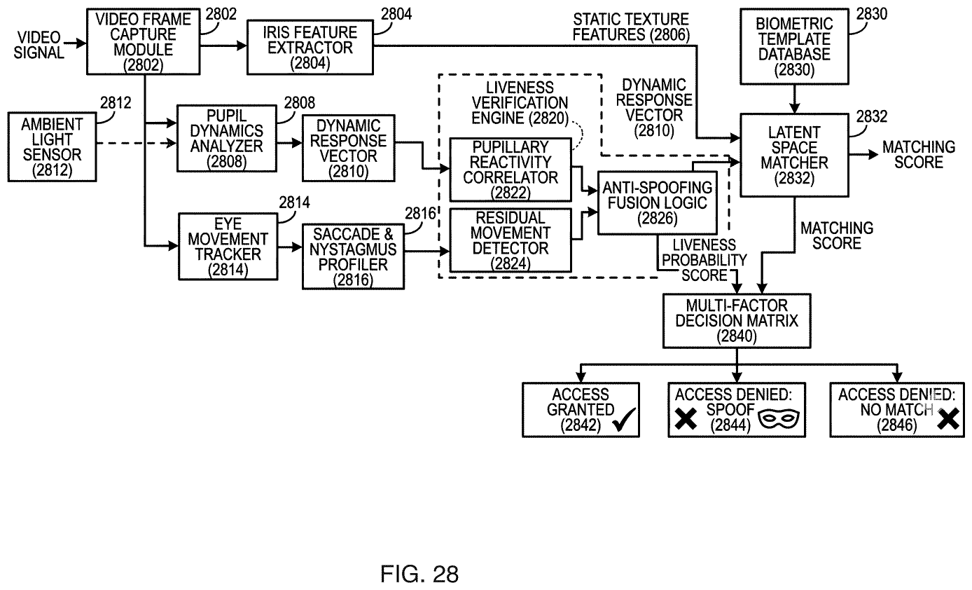

FIG. 28 is a block diagram illustrating a biometric authentication or identification subsystem that uses iris features, pupil dynamics, and eye-movement features to generate an authentication decision, in accordance with embodiments of the present disclosure. In some cases, the biometric authentication or identification subsystem shown in FIG. 28 may comprise a liveness assessment subsystem. In some cases, the subsystem shown in FIG. 28 may comprise a biometric authentication and liveness assessment subsystem 2800 that may be implemented by one or more electronic processors of a pupillometry device (e.g., processing unit 106 of device 100) using the same video sequence captured for computational pupillometry. In embodiments, the subsystem 2800 generates (i) a matching score indicative of similarity between acquired biometric features and stored biometric templates and (ii) a liveness probability score indicative of whether the acquisition is consistent with a living subject, and produces an authentication outcome based on such outputs.

As indicated by the arrows in FIG. 28, a video frame capture module 2802 receives a video signal representing a sequence of frames of an eye acquired during a pupillometry protocol and provides video frames to an iris feature extractor 2804, a pupil dynamics analyzer 2808, and an eye movement tracker 2814. In embodiments, the iris feature extractor 2804 isolates an iris region in one or more frames and outputs static texture features 2806 comprising one or more iris feature descriptors derived from the iris region. In embodiments, the pupil dynamics analyzer 2808 measures pupil diameter over time and outputs a dynamic response vector 2810 comprising one or more pupillary response parameters and/or time-series response features, including one or more of baseline diameter, minimum diameter, constriction amplitude, latency, constriction velocity, dilation velocity, and recovery dynamics. In embodiments, an ambient light sensor 2812 provides ambient illumination context to the pupil dynamics analyzer 2808 and/or other processing blocks, and the ambient illumination context is used to normalize, condition, and/or quality-gate the dynamic response vector 2810.

In embodiments, the eye movement tracker 2814 determines one or more eye-movement features from the video frames and provides such features to a saccade and nystagmus profiler 2816. In embodiments, the profiler 2816 outputs one or more eye-movement signatures and/or summary metrics, including one or more of fixation stability, saccade count, saccade amplitude, saccade velocity, and nystagmus signatures.

In embodiments, a liveness verification engine 2820 receives the dynamic response vector 2810 and one or more eye-movement outputs and generates a liveness probability score. In embodiments, a pupillary reactivity correlator 2822 evaluates whether the dynamic response vector 2810 exhibits an expected temporal relationship to a controlled light stimulus protocol, based on a known stimulus timing and/or a detected stimulus onset derived from the acquisition. In embodiments, a residual movement detector 2824 evaluates whether residual physiological eye movements are present in the acquisition using outputs of the saccade and nystagmus profiler 2816. In embodiments, anti-spoofing fusion logic 2826 combines outputs of the pupillary reactivity correlator 2822 and the residual movement detector 2824 to generate the liveness probability score.

In embodiments, a biometric template database 2830 stores one or more enrolled biometric templates associated with authorized users and/or patient identifiers. In embodiments, a latent space matcher 2832 receives the static texture features 2806 and optionally one or more portions of the dynamic response vector 2810, and compares the received features to one or more stored templates in the biometric template database 2830. In embodiments, the latent space matcher 2832 generates a feature embedding (latent representation) for at least one of the static texture features 2806 and the dynamic response vector 2810 and computes a similarity measure between the embedding and at least one stored template embedding, thereby producing a matching score.

In embodiments, a multi-factor decision matrix 2840 receives the matching score and the liveness probability score and determines an authentication outcome. In embodiments, access is granted 2842 when the matching score satisfies a match criterion and the liveness probability score satisfies a liveness criterion. In embodiments, access is denied as spoof 2844 when the matching score satisfies the match criterion and the liveness probability score fails the liveness criterion. In embodiments, access is denied as no match 2846 when the matching score fails the match criterion. In embodiments, the authentication outcome is used to associate a pupillometry measurement record with a correct patient record and/or to control access to stored pupillometry measurement records and synchronized clinical records.”

In embodiments, the system supports protocol adherence optimization by determining that testing frequency is below a prescribed protocol and generating user notifications to prompt recapture at a target cadence.

### Technical Definitions

As used herein, the term stimulus illumination refers to light emitted by a controlled source (e.g., the light emitter 104) that is applied according to a stimulus protocol to elicit a pupillary response. The term ambient illumination refers to environmental illumination incident on the eye that is present before and during the stimulus protocol and that is not solely attributable to the controlled stimulus illumination. The term estimated ambient illumination refers to a computed estimate of ambient illumination derived from sensor measurements, image data, or both. The term computational model refers to one or more algorithms that map one or more input features to one or more outputs, including a parameterized function, a calibrated lookup table, a regression model, and/or a machine learning model trained using labeled or calibrated data.

As used herein, the term computer vision refers to algorithmic and model-based techniques for extracting measurements from images or video, including deep learning and machine-learning approaches. Non-limiting examples include object-detection models (e.g., models producing bounding boxes for pupil and/or iris regions), segmentation models (e.g., models producing pixel-level masks or contours of ocular structures), convolutional neural networks, and transformer-based vision models. Such techniques are used to compute eye-movement metrics, pupil geometry, and related features from video sequences.

As used herein, the term normalized pupil reactivity score (also referred to herein as a PuRe score) refers to a scalar value computed from one or more pupillary response parameters and ambient illumination context to provide a reactivity measure that is interpretable across differing ambient illumination conditions. In embodiments, the PuRe score is computed using a function (including a saturation-type mapping) and/or a machine learning model. In embodiments, the PuRe score is scaled to a defined range (e.g., from 0 to a maximum value PuReMAX). As used herein, the term unreactive refers to a classification in which a measured pupillary constriction (e.g., CAMP and/or Δ) does not exceed a threshold during a response window, where the threshold can depend on ambient illumination and baseline pupil diameter (INIT).

As used herein, the term substantially light-invariant refers to an output score, classification, or other derived value that, when evaluated across a set of measurements spanning multiple ambient illumination conditions, exhibits limited dependence on ambient illumination. In embodiments, limited dependence is demonstrated by an absolute Spearman rank correlation coefficient between the output score and log₁₀(lux) (or another monotone transform of illumination) that does not exceed a threshold (e.g., |ρ|≤0.15) when evaluated across a practical operating range (e.g., about 0.1 to about 3000 lux) and/or by other statistical bounds. Such thresholds are provided as examples and can be selected based on application context, evaluation dataset, and clinical workflow.

As used herein, the term synchronization (or synchronized operation) in a multi-device context refers to coordination in which a first pupillometry device stores one or more measurements locally and exchanges one or more records with a server such that a second pupillometry device can access consistent patient records across care locations. Unless explicitly stated otherwise, synchronization does not require strict clock-phase synchronization between devices and can be implemented using networked data coordination, conflict resolution, and audit logging.

As used herein, normative Data (e.g., normative data 706 in FIG. 2 below) comprises one or more normative datasets derived from pupillometry measurements collected from reference subjects (e.g., neurologically healthy subjects) across multiple ambient illumination conditions. In embodiments, the normative data are measured by acquiring pupillometry video sequences under controlled protocols at different illumination levels and extracting corresponding pupillary response parameters. The normative data are used to derive illumination-dependent relationships that support parameter adjustment and normalization. In embodiments, the normative data and/or parameters derived therefrom are stored in non-volatile memory of the computing device and/or in a remote server and accessed by the Light-Dependent Parameter Adjuster 702 during score computation.

As used herein, “encodes” means that the calibration data 705 stores, in a machine-readable form, one or more representations of the empirically determined relationships (e.g., as a lookup table, coefficients for one or more equations, a mapping function, and/or trained model parameters).

As used herein, geometrically corrected frame data refers to video frames that have been spatially registered into a common coordinate system to compensate for detected inter-frame motion.

Pupillometry System with Ambient Light Correction

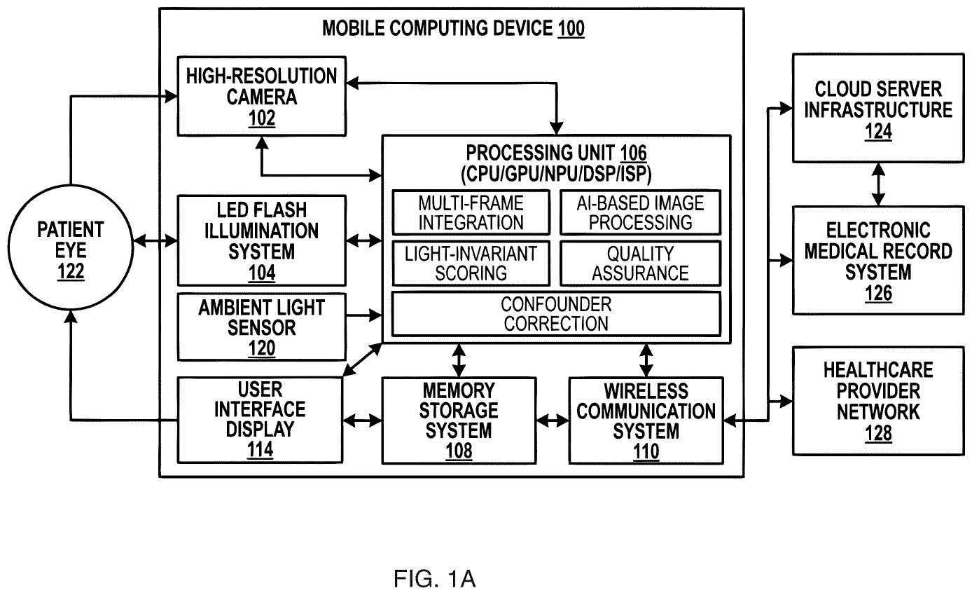

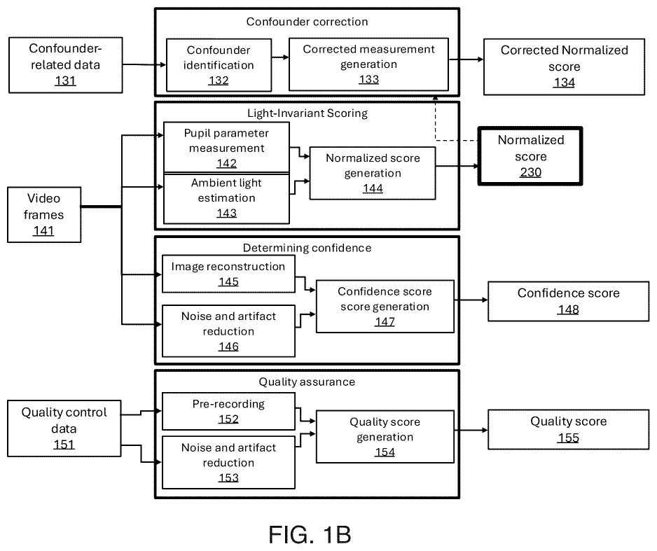

FIG. 1A illustrates an embodiment of a computational pupillometry system. In embodiments, a computing device 100 (e.g., a mobile computing device such as a smart phone) includes an camera or imaging device 102 (e.g., a high-resolution camera) configured to capture video frames of an eye of a patient or subject 122 during pupillometry assessment that may comprise a controlled stimulus protocol generated by a light emitter 104. The computing device 100 may further comprise a processing unit 106 and a data storage system or memory 108, e.g., a non-transitory memory. The processing unit 106 may comprise a hardware processor (e.g., a CPU, a GPU, an NPU, a DSP, an ISP, and other types of processors) configured to execute machine-readable instructions to (i) acquire and store one or more video sequences using the imaging device 102, (ii) extract pupillary measurements from the video sequences received from the imaging device 102, (iii) measure and/or estimate ambient illumination conditions and (iv) compute and output one or more scores and classifications related to or indicative of a pupillary function. In some embodiments, the computing device 100 may comprise or can be in communication with an ambient sensor 120 configured to generate a sensor signal indicative of a level light in an environment surrounding the computing device 100. In some embodiments, the processing unit 106 may measure and/or estimate the ambient illumination conditions (e.g., a level of ambient light) using one or both the sensor signal generated by the ambient light sensor 120 and an image or a video frame received from the camera 102. In some cases, the processing unit 106 may estimate the level of ambient light by processing the image data associated with the image or the video). In various implementations, the level of ambient light may comprise illuminance (e.g., measured in Lux), irradiance (e.g., measured in optical power/unit area), radiant intensity (e.g., power per unit solid angle), or intensity, of ambient light. However, embodiments are not so limited and other quantitative measures of light.

In some embodiments, the computing device 100 may comprise a user interface 114 (e.g., a touch screen display) configured to received user inputs from a user and a wireless communication system 110 configured to communicate with one or more of a cloud server infrastructure 124, an electronic medical record system 126, a healthcare provider network 128, or another remote computing system. In some embodiments, the computing device 100 may further comprise an illumination system 104 (e.g., a light emitting diode flash illumination system) configured to provided controlled illumination to patient's eye 122. For example, the processing unit 106 may use the illumination system 104 to provide a predetermined or calculated amount of light within a specified wavelength to the patient's eye 122. In some cases, the processing unit 106 may determine an amount of light provided by the illumination system 104 to the patient's eye 122 based at least in part on one or more of a measured level of ambient light, data stored in the data storage 108, a sensor signal received from the ambient light sensor 120, data received via a user interface (e.g., user interface 114), data received via the wireless communication system 110.

FIG. 1B illustrates example input data, output data, and processes flow for selected blocks in the processing unit 106 of the computing device 100 or computational pupillometry system shown in FIG. 1A. In some examples, these processes may be by the processing unit 106 by executing machine-readable instructions stored in the data storage system 108. In embodiments, a plurality of video frames 141 (e.g., captured using camera 102) may be provided to a light-invariant scoring pipeline that may include a pupil parameter measurement process 142 and ambient light estimation process 143, and can be configured to generate a normalized score 230 via normalized score generation process 144. In embodiments, the video frames 141 may be also provided to a confidence determination pipeline that may include an image reconstruction process 145 and a noise and artifact reduction process 146, and may be configured to generate a confidence score 148 via a confidence score generation process 147. In embodiments, the confidence score 148 may comprise a quantitative reliability metric associated with one or more pupillary response parameters and/or the normalized score 230. In some cases, the confidence score 148 may be determined based on one or more of a reconstruction fidelity, a noise metric, an artifact metric, and pupil boundary detection quality. In embodiments, quality control data 151 may be provided to a quality assurance pipeline that may include a pre-recording process 152 and noise and artifact reduction process 153 and may be configured to generate a quality score 155 via quality score generation 154. In embodiments, confounder-related data 131 may be provided to a confounder correction pipeline that may include a confounder identification process 132 and a corrected measurement generation process 133, and may be configured to generate a corrected normalized score 134. In some embodiments, the corrected measurement generation process 133 may receive the normalized score 230 from the light-invariant scoring pipeline and generate the corrected normalized score 134 based on the normalized score 230.

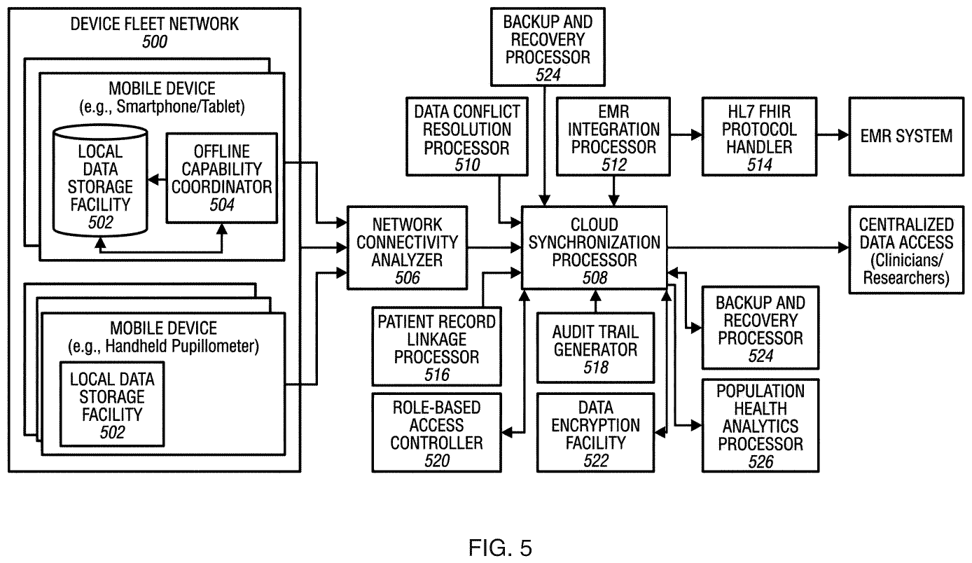

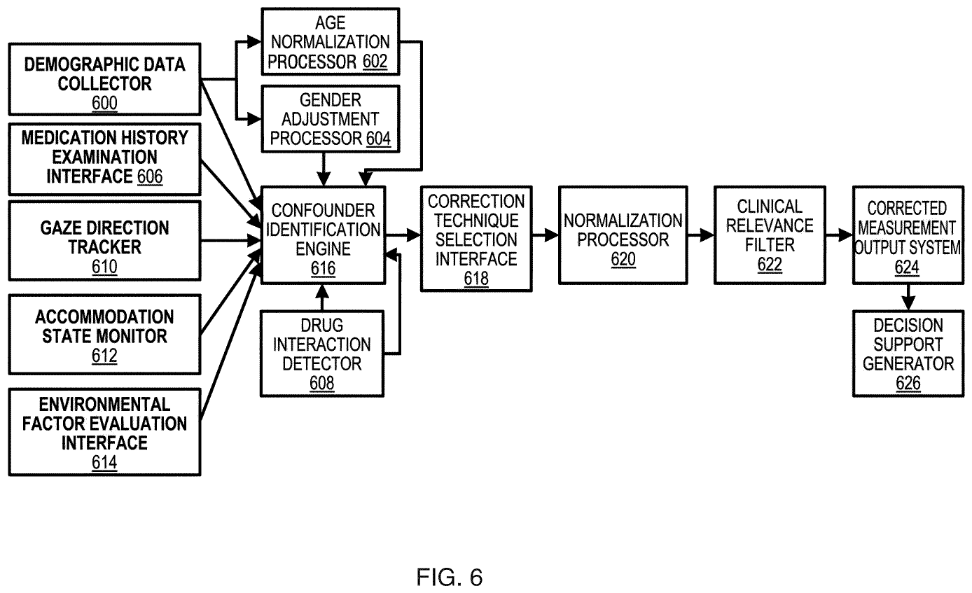

Regarding FIG. 1, a mobile computing device (e.g., a smartphone or tablet) 100 represents a sophisticated computational platform specifically engineered to overcome the fundamental technical limitations inherent in conventional pupillometry systems through advanced error detection, compensation mechanisms, and multi-frame processing techniques, thereby transforming traditional measurement challenges into computational advantages. In embodiments, FIG. 1 provides an overall architecture, and additional detail about example subsystems is described with respect to FIG. 2 (light-aware scoring pipeline), FIG. 3 (multi-frame integration), FIG. 4 (quality control and gating), FIG. 5 (synchronization), and FIG. 6 (confounder correction).

In embodiments, as indicated by the arrows in FIG. 1, the camera 102 captures video frames of the patient eye 122 and provides the frames to the processing unit 106, which stores frames and derived parameters in memory 108 and controls the light emitter 104 according to a stimulus protocol. The processing unit 106 also receives ambient illumination input from an ambient light sensor 120 and uses such context with video-derived pupillary response parameters to generate a normalized score and/or classification, which is presented on the user interface 114 and optionally stored and/or exported to an electronic medical record system 126 and/or synchronized to a cloud server 124 through a wireless communication system 110 for access by a healthcare provider network 128.

In embodiments, the mobile computing device 100 implements a comprehensive error detection and compensation architecture that addresses specific technical problems that traditional dedicated hardware systems cannot solve due to processing limitations and architectural constraints. The mobile computing device 100 comprises heterogeneous processing cores including central processing units (CPUs) that operate at, for example, 2.4-3.8 GHZ, graphics processing units (GPUs) with, for example, 1000-2000 computational cores, and neural processing units (NPUs) that deliver, for example, 15-20 trillion operations per second (TOPS), though other specifications such as 8-25 TOPS for NPUs, 1.8-4.2 GHz for CPUs, or 500-3000 GPU cores may be utilized depending on specific device implementations and computational requirements. This distributed processing architecture enables real-time execution of sophisticated error detection techniques that continuously monitor measurement quality through multi-parameter analysis, including temporal consistency validation, spatial coherence assessment, and physiological plausibility verification.

In embodiments, the high-resolution camera 102 constitutes a precision imaging subsystem specifically engineered to identify and compensate for measurement artifacts that compromise traditional pupillometry systems. The high-resolution camera 102 interfaces with the processing unit 106 through high-bandwidth communication pathways that support, for example, transfer rates of 1-4 Gbps, enabling real-time artifact detection through frame-by-frame analysis of pixel intensity distributions, edge detection techniques, and temporal consistency monitoring. However, other transfer rates, such as 500 Mbps to 8 Gbps, may be utilized based on device capabilities and processing requirements.

The processing unit 106 executes multi-stage artifact detection techniques through the high-resolution camera 102 that identify and categorize measurement errors including: (1) corneal reflection artifacts that the system detects through specular analysis techniques that distinguish reflections from genuine pupil boundaries by analyzing intensity gradients exceeding, for example, 80-90% of maximum pixel values within localized regions, though other thresholds such as 70-95% may be applied; (2) motion blur artifacts that the system identifies through edge sharpness analysis using Sobel gradient operators with threshold detection below, for example, 0.3 normalized edge strength, though other thresholds ranging from 0.2-0.5 may be utilized; (3) occlusion artifacts from eyelids or eyelashes that the system detects through boundary continuity analysis that identifies gaps in pupil circumference exceeding, for example, 15-20% of total boundary length, though other percentages ranging from 10-25% may be implemented; and (4) lighting variation artifacts that the system detects through histogram analysis identifying intensity changes exceeding, for example, 20-30% between consecutive frames, though other change thresholds ranging from 15-35% may be applied.

In embodiments, the processing unit 106 implements corneal reflection analysis to detect, quantify, and mitigate a specular flash reflection (also referred to herein as a glint) that can obscure the pupil region and/or indicate suboptimal acquisition geometry. In some embodiments, the corneal surface is modeled as an approximately spherical convex reflective surface with a radius of curvature R (e.g., about 7-8 millimeters). The system treats the light source (e.g., an LED flash) as an object at a distance dₒ from the corneal apex (e.g., about 15-30 millimeters) and computes a virtual image distance dᵢ using a spherical-mirror equation 1/f=1/dₒ+1/dᵢ, where f=−R/2 for a convex mirror. By way of non-limiting example, with R≈7.8 mm and dₒ≈25 mm, the virtual image can be located at dᵢ≈−3.5 mm relative to the corneal surface. A camera lens (e.g., focal length of about 3-6 mm at a camera-to-cornea distance of about 20-40 mm) images the virtual source to produce an approximately circular specular highlight in the captured video frames. In embodiments, the processing unit 106 uses the corneal reflection analysis to: (i) detect and mask reflection pixels prior to pupil boundary fitting or segmentation; (ii) compute a quality indicator reflecting whether the reflection overlaps a pupil region of interest above a threshold; and/or (iii) estimate acquisition geometry (e.g., camera-to-eye distance and/or incidence angle) using relative placement between a reflection centroid and a pupil center. In some embodiments, the estimated geometry is used to guide the user (e.g., adjust distance and/or angle) and/or to adjust stimulus parameters.

In embodiments, corneal reflection detection can be performed using intensity-threshold segmentation of saturated pixels, morphological filtering to enforce compactness and circularity, Hough-transform circle detection, and/or temporal consistency checks across multiple frames. In embodiments, the system trains a machine learning model using datasets in which reflections are manually annotated to produce labels (e.g., masks, contours, or bounding boxes). Non-limiting examples include object detection models (e.g., YOLO) that output reflection bounding boxes, segmentation models (e.g., U-Net, Mask R-CNN, or DeepLab) that output reflection masks, and transformer-based vision models that output reflection segmentation and/or confidence values. In embodiments, the reflection model is trained jointly with, or as an auxiliary head to, a pupil segmentation model, and its output is used to exclude reflection regions from subsequent pupillometry parameter estimation.

The processing unit 106 executes sophisticated compensation techniques that correct artifacts identified by the system through multiple technical approaches, which traditional systems cannot implement due to computational limitations. In embodiments, the processing unit 106 utilizes specular reflection compensation through inpainting techniques based on partial differential equation solvers that reconstruct occluded pupil regions by analyzing surrounding pixel patterns and temporal consistency across multiple frames. The processing unit 106 implements edge-preserving smoothing filters including bilateral filtering with spatial sigma parameters of, for example, 5-15 pixels and intensity sigma parameters of, for example, 20-50 intensity levels, though other parameters such as spatial sigma ranging from 3-25 pixels or intensity sigma ranging from 10-80 may be utilized based on specific image characteristics and processing requirements.

The processing unit 106 executes motion compensation techniques that implement sophisticated multi-frame registration methods using phase correlation techniques, achieving sub-pixel accuracy, for example, of ±0.1 pixels through Fourier domain analysis. Other accuracy levels, ranging from ±0.05 to ±0.2 pixels, may be achieved based on processing capabilities and algorithmic implementations. These techniques specifically address the technical problem of handheld device instability by transforming natural micro-movements into advantageous data sources through parallax-based super-resolution processing. In embodiments, the processing unit 106 analyzes displacement vectors between consecutive frames using optical flow techniques, including Lucas-Kanade methods with pyramid levels of, for example, 3-5 levels and window sizes of, for example, 15×15 to 31×31 pixels, though other configurations, such as 2-7 pyramid levels or window sizes ranging from 11×11 to 41×41 pixels may be implemented.

The processing unit 106 implements multi-frame super-resolution processing that solves the fundamental technical problem of limited spatial resolution that constrains traditional pupillometry accuracy. Unlike some of the existing systems that rely on single-frame analysis, the processing unit 106 combines information from, for example, 50-200 video frames to reconstruct higher-resolution pupil boundary information with effective resolution enhancement factors of, for example, 2×-4× beyond native sensor capabilities, though other frame counts ranging from 20-500 frames or enhancement factors ranging from 1.5×-6× may be achieved based on computational resources and quality requirements. This technical approach leverages sub-pixel shifts caused by natural micro-movements to sample the pupil boundary at multiple spatial positions, enabling the reconstruction of pupil contours with precision of, for example, ±0.025 mm, compared to the ±0.03-0.10 mm accuracy limitations of traditional infrared systems. However, precision levels ranging from ±0.015 to ±0.035 mm may be achieved.

In embodiments, the LED flash illumination system 104 implements adaptive error correction mechanisms that solve the technical challenge of ambient light variability through real-time compensation techniques. The LED flash illumination system 104 incorporates flash precompensation techniques that anticipate changes in camera exposure and minimize recording artifacts through the predictive adjustment of illumination parameters. The processing unit 106 calculates optimal flash intensity using distance-corrected inverse square law calculations combined with ambient light measurements to maintain consistent pupillary stimulus energy of, for example, 0.1-1.0 photopic cd·s/m², though other energy levels such as 0.05-2.0 cd·s/m² may be utilized based on clinical requirements and safety considerations. In embodiments, the processing unit 106 controls one or more stimulus parameters of the light source 104, including intensity, duration, pulse waveform (e.g., a single pulse or a pulse train), and/or spectral characteristics. In embodiments, the stimulus parameters are selected based on ambient illumination and/or acquisition geometry (e.g., distance estimates from image analysis and/or corneal reflection analysis), and can be adjusted to maintain a desired stimulus at the eye. In embodiments, different stimulus parameters are applied for different recording intervals (e.g., pre-stimulus, stimulus, and post-stimulus intervals). In embodiments, a stimulus protocol comprises two or more distinct illumination levels (e.g., a baseline illumination level during a pre-stimulus interval, a stimulus illumination level during a stimulus interval, and a post-stimulus illumination level during a post-stimulus interval), and the post-stimulus illumination level can differ from the baseline illumination level. In some embodiments, the stimulus is delivered at a fixed, predetermined intensity, temporal profile, and geometry. In some cases, ambient illumination estimates may be used to adjust stimulus parameters (e.g., increasing stimulus intensity in bright environments above a threshold) to probe residual reactivity, subject to hardware limits. Such adaptive control is non-essential and may be omitted.

The LED flash illumination system 104 specifically addresses the technical problem of stimulus standardization across diverse environmental conditions by implementing baseline flash techniques with intensity modulation techniques that adjust output parameters based on real-time analysis of environmental factors. In embodiments, the light source 104 is additionally configured to emit a weak baseline illumination (also referred to herein as a base flash) during at least a portion of a pre-stimulus interval. In embodiments, the baseline illumination has an intensity at the eye that is less than about 50 lux, and in some embodiments less than about 30 lux, thereby enabling visible-light video capture in near-darkness or low light condition without substantially constricting the pupil prior to the light stimulus. In some embodiments, low-light condition may comprise an ambient illumination less than about 2 lux, less than about 1 lux, less than about 0.5 lux or smaller values. In embodiments, the baseline illumination provides a standardized background level that improves stability of pre-stimulus measurements (e.g., INIT and baseline intensity statistics) and reduces variability in subsequent response dynamics by reducing abrupt transitions from darkness to a bright stimulus. In embodiments, the baseline illumination is produced by the same light source used for the stimulus or by a separate light source, and is delivered as a continuous low-intensity output and/or as a sequence of pulses with a duty cycle selected to maintain a desired mean illuminance. In embodiments, the device additionally applies a post-stimulus illumination level during at least a portion of the post-stimulus interval (e.g., to improve measurement of recovery dynamics), and the post-stimulus illumination level differs from the baseline illumination level (e.g., higher or lower). the processing unit 106 analyzes ambient light measurements from the ambient light sensor 120. It implements compensation functions including logarithmic scaling relationships where flash intensity scales according to I_flash=I_base×(1+a×log₁₀(L_ambient/L_reference)), where a represents an adaptation coefficient of, for example, 0.2-0.8, and L_reference represents a standardized illumination level of, for example, 100 lux, though other coefficient ranges such as 0.1-1.0 or reference levels ranging from 50-200 lux may be utilized.

In non-limiting examples, pre-stimulus baseline illumination is below about 50 lux (e.g., about 20 lux or near 0 lux), stimulus illumination exceeds about 300 lux (e.g., up to about 800 lux depending on hardware), and post-stimulus illumination returns to the baseline level or an intermediate level to probe recovery.

The processing unit 106 implements sophisticated temporal neural network architectures that solve the technical problem of robust pupil tracking under challenging conditions that defeat traditional segmentation approaches. In embodiments, the processing unit 106 executes 3D convolutional neural networks with architectures including, for example, 5-15 convolutional layers, temporal kernel sizes of 3×3×3 to 7×7×7, and feature map channels ranging from 32-512 per layer, though other configurations such as 3-20 layers, kernel sizes ranging from 2×2×2 to 9×9×9, or feature channels ranging from 16-1024 may be implemented. These networks analyze spatiotemporal patterns across video sequences to distinguish genuine pupillary features from transient artifacts through feature representations that the system trains on datasets exceeding, for example, 1 million annotated pupil images. However, other dataset sizes, ranging from 500,000 to 2 million images, may also be utilized.

The neural network processing specifically implements attention mechanisms that dynamically focus computational resources on pupil regions while suppressing artifact-prone areas. In embodiments, the attention techniques utilize self-attention mechanisms with multi-head configurations including, for example, 8-16 attention heads and embedding dimensions of, for example, 256-1024, though other configurations such as 4-32 attention heads or embedding dimensions ranging from 128-2048 may be implemented, enabling the system to assign differential importance weights to spatial regions and temporal frames based on measurement quality indicators.

The processing unit 106 executes temporal averaging techniques that implement sophisticated noise reduction techniques that exceed simple frame averaging through weighted integration schemes. In embodiments, the processing unit 106 applies temporal weights based on frame quality metrics, including sharpness scores calculated using Laplacian variance methods, stability scores derived from optical flow magnitude analysis, and illumination consistency scores computed through histogram correlation analysis. The system assigns reduced weights in the averaging process to frames with quality scores below predetermined thresholds, such as 0.7 on a normalized scale. Other thresholds, ranging from 0.5 to 0.9, may be applied based on specific quality requirements.

The processing unit 106 implements parallax-based artifact rejection techniques that solve the technical problem of distinguishing genuine pupillary features from spurious reflections through depth-based analysis. In embodiments, these techniques analyze differential motion patterns between features at different optical depths, where the system classifies corneal reflections exhibiting motion vectors with magnitudes exceeding, for example, 1.5× pupil boundary motion vectors as artifacts and excludes them from boundary detection techniques. However, other threshold ratios, ranging from 1.2 to 2.0 times the reference value, may be utilized based on measurement conditions.

The processing unit 106 implements error propagation analysis, providing comprehensive uncertainty quantification that traditional systems cannot offer due to computational constraints. In embodiments, the processing unit 106 calculates measurement uncertainties through Monte Carlo sampling methods with, for example, 1000-10000 iterations, propagating uncertainties from individual frame measurements through multi-frame integration techniques to provide final measurement confidence intervals of, for example, ±0.015-0.035 mm depending on environmental conditions and measurement quality, though other iteration counts ranging from 500-20000 or confidence intervals ranging from ±0.010-0.050 mm may be achieved.

In embodiments, the memory storage system 108 implements sophisticated data management architectures that support real-time error detection and compensation processing. The memory storage system 108 maintains high-speed buffers for temporal processing including circular frame buffers of, for example, 2-8 GB capacity that store recent video frames with timestamps accurate to, for example, 1 microsecond precision for temporal correlation analysis, though other buffer sizes ranging from 1-16 GB or timestamp precision ranging from 0.1-10 microseconds may be implemented based on processing requirements.

The processing unit 106 executes quality validation techniques that implement multi-stage verification processes that ensure measurement reliability through comprehensive analysis of physiological plausibility, temporal consistency, and measurement precision indicators. In embodiments, physiological plausibility validation verifies that pupil diameter measurements fall within expected ranges of, for example, 1.5-8.0 mm and that constriction velocities remain within physiological limits of, for example, 0.5-6.0 mm/s maximum constriction velocity, though other diameter ranges such as 1.0-9.0 mm or velocity limits ranging from 0.3-8.0 mm/s may be applied based on patient populations.

In embodiments, the wireless communication system 110 enables distributed error detection and validation through cloud-based comparison techniques that leverage aggregated measurement data for outlier detection. The wireless communication system 110 transmits measurement metadata, including quality indicators, environmental conditions, and processing parameters, to cloud server infrastructure 124 for population-based validation analysis with response times of, for example, less than 500 milliseconds, though other response times ranging from 100-1000 milliseconds may be achieved based on network conditions and computational load.

The processing unit 106 implements advanced calibration mechanisms that provide continuous system validation and drift correction capabilities, which traditional dedicated hardware cannot achieve due to its limited onboard processing. In embodiments, the system utilizes calibration techniques that include reference patterns, such as synthetic pupil boundaries generated through computer graphics techniques and known geometric calibration targets. These techniques verify measurement accuracy with validation precision of, for example, ±0.005 mm against reference standards, although other precision levels, ranging from ±0.003 to ±0.010 mm, may also be achieved.

In embodiments, the ambient light sensor 120 integrates with sophisticated light estimation techniques that address the technical challenge of accurately characterizing the environment across dynamic lighting conditions. The processing unit 106 combines direct sensor measurements with computer vision-based light estimation, utilizing statistical analysis of video frame characteristics, including root-mean-square intensity values, histogram percentile analysis, and spatial gradient magnitude assessments, to achieve ambient light estimation accuracy of, for example, ±10% across illumination ranges from 0.1 to 50,000 lux. However, other accuracy levels, such as ±5-15%, or illumination ranges from 0.01 to 100,000 lux, may be achieved based on sensor capabilities and processing techniques.

In embodiments, the patient eye 122 represents the biological measurement target that interfaces with the system through controlled optical interactions with both the high-resolution camera 102 and the LED flash illumination system 104. The patient's eye 122 provides physiological responses, including pupillary light reflexes, that form the basis for advanced computational analysis performed by the processing unit 106, enabling the assessment of neurological function, autonomic nervous system activity, and other clinically relevant parameters through standardized measurement protocols.

The measurement process specifically addresses technical challenges in extracting accurate pupillary response parameters from biological systems by implementing advanced signal processing techniques that compensate for natural variations in ocular anatomy, physiological responses, and environmental measurement conditions. In embodiments, the system analyzes multiple physiological parameters, including baseline pupil diameter, constriction amplitude, response latency, and temporal dynamics, to generate comprehensive clinical assessments with quantified confidence intervals.

In embodiments, a pupillometry device 100 transmits measurement results (and in some embodiments associated video and/or derived features) to cloud server infrastructure 124 through secure wireless communication systems. The cloud server infrastructure 124 provides scalable computational resources and data management capabilities that store and process the uploaded records, thereby enabling synchronization such that a second pupillometry device can retrieve consistent patient records across institutional environments. In embodiments, the cloud server infrastructure 124 performs advanced analytics, population health monitoring, and system performance optimization, and supports continuous improvement of one or more models through analysis of aggregated (and privacy-protected) clinical data.

In embodiments, the cloud server infrastructure 124 implements data processing pipelines that perform computationally intensive analyses exceeding the capabilities of an individual pupillometry device while maintaining real-time responsiveness for clinical applications. In embodiments, the cloud server infrastructure 124 returns to the pupillometry device (and/or to a second pupillometry device) one or more derived outputs and/or updated parameters or models (e.g., calibration tables or scoring parameters). In embodiments, the cloud server infrastructure 124 facilitates secure data exchange between healthcare institutions, including implementing robust security protocols, privacy protections, and audit trail maintenance for regulatory compliance.

In embodiments, the electronic medical record system 126 represents a healthcare information infrastructure that integrates with the pupillometry platform through standardized healthcare data protocols, including HL7 FHIR and other interoperability standards. The electronic medical record system 126 enables seamless incorporation of measurement data into clinical workflows and patient records through bidirectional data exchange protocols that maintain data integrity while supporting comprehensive clinical documentation and longitudinal patient monitoring across multiple care episodes with transaction processing rates of, for example, 1000+ messages per minute. However, other rates, ranging from 100 to 10,000 messages per minute, may also be achieved.

The integration between the mobile computing device 100 and the electronic medical record system 126 specifically addresses technical challenges in healthcare data interoperability by implementing standardized data formats, secure transmission protocols, and automated data validation mechanisms that ensure accurate clinical documentation while reducing administrative burden on healthcare providers. In embodiments, this integration enables real-time clinical decision support and the automated generation of clinical reports based on pupillometry measurement data, with customizable formatting for different healthcare specialties. The data synchronization latency is typically less than 5 seconds, although other latency ranges, such as 1-30 seconds, may also be achieved.

In embodiments, a pupillometry device 100 used by a healthcare provider uploads measurement records to the cloud server infrastructure 124 and, in return, can receive synchronized patient data and/or updated protocols or model parameters. In embodiments, a second pupillometry device used by another provider (e.g., in a different care setting) retrieves the same synchronized patient records from the cloud server infrastructure 124. In embodiments, aggregated and privacy-protected data support distributed clinical research, population health analytics, and continuous improvement of measurement protocols.

The healthcare provider network 128 specifically addresses technical challenges in healthcare system interoperability and care coordination by implementing standardized communication protocols, shared data governance frameworks, and collaborative quality assurance mechanisms. In embodiments, the healthcare provider network 128 enables patient tracking across multiple care settings while maintaining data security and privacy through advanced encryption, access control, and audit logging mechanisms that comply with healthcare regulatory requirements. In embodiments, a first pupillometry device used at a first healthcare facility uploads a patient record to the cloud server infrastructure 124, and a second pupillometry device used at a second healthcare facility retrieves the synchronized patient record, thereby supporting multi-site data synchronization across, for example, 10 or more facilities, in some cases, substantially simultaneously. However, other scales, such as 5-100+ facilities, may also be supported.

The interconnected system architecture enables sophisticated bidirectional data flow patterns, where the mobile computing device 100 functions as both an autonomous measurement platform and an integrated component within a comprehensive healthcare information ecosystem (e.g., substantially simultaneously). In embodiments, pupillary measurement data captured by the high-resolution camera 102 and processed by the processing unit 106 through advanced AI models becomes immediately available through the user interface 114 (e.g., via a display of the user interface 114) for real-time clinical decision-making while being automatically transmitted through the wireless communication system 110 to the cloud server infrastructure 124 for integration with the electronic medical record system 126 and broader healthcare provider network 128.

This comprehensive system architecture transforms raw video data into clinically actionable insights through sophisticated computational pipelines that the processing unit 106 executes. The system enables superior clinical performance compared to traditional dedicated hardware while providing unprecedented accessibility and integration capabilities that transform pupillometry from specialized equipment-dependent procedures to ubiquitous point-of-care assessments available across the entire healthcare continuum with measurement precision of, for example, ±0.025 mm and light-invariant scoring across ambient conditions ranging from, for example, 4-1,200 lux, though other precision levels ranging from ±0.015-0.035 mm or ambient ranges from about 0.1-3000 lux (and in some embodiments up to about 10,000 lux) may be achieved based on specific implementation requirements and clinical applications.

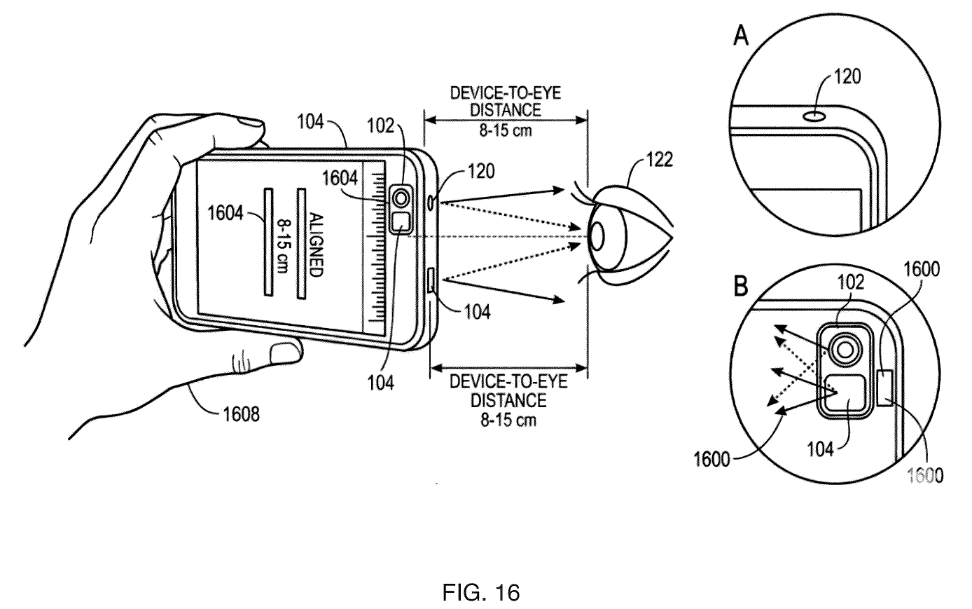

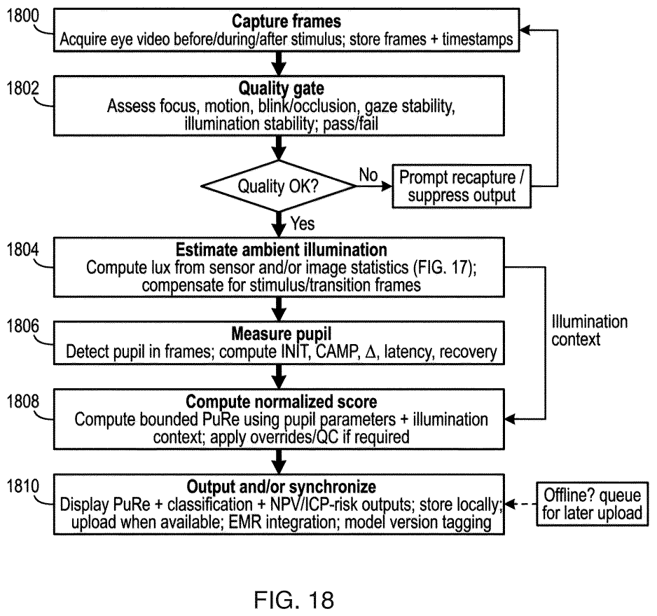

In embodiments, FIG. 16 illustrates an example handheld pupillometry acquisition setup. In embodiments, the mobile computing device 100 is positioned relative to the subject eye 122 using an eye cup 1602 and/or a distance indicator 1604 to promote consistent acquisition geometry. In embodiments, ambient illumination is measured using an ambient light sensor 120 and/or an auxiliary photodiode module 1600. In embodiments, an ambient shield 1606 reduces stray illumination and reflections during acquisition, and an operator hand 1608 positions the device during capture. In embodiments, the acquisition setup of FIG. 16 supports the processing and scoring pipeline described with respect to FIG. 2 and the end-to-end sequence of operation described with respect to FIG. 18.

In embodiments, as indicated by the arrows in FIG. 16, the pupillometry device establishes acquisition geometry using the eye cup 1602 and/or the distance indicator 1604, captures a video sequence of the subject eye 122, and concurrently obtains ambient illumination context using the ambient light sensor 120 and/or the photodiode module 1600. In embodiments, the captured video and illumination context are provided to downstream modules to compute one or more pupillary response parameters, a normalized score, and/or one or more classifications.

FIG. 2 illustrates an embodiment of a light-aware scoring pipeline. In some embodiments, the light scoring pipeline show in FIG. 2 may be implemented by the computing device 100 (e.g., by the processing unit 106) by executing machine readable instructions stored in the data storage system 108. In embodiments, the system (e.g., the computing system 100) captures a video sequence comprising the patient's eye 122, using a video frame processor 200. In some cases, the video sequence can include one or more frames acquired before, during, and after a stimulus (e.g., a stimulus provided by the processing unit 106 using the illumination system 104). The system (e.g., the processing unit 106) may process the video sequence to determine one or more pupillary response parameters (e.g., baseline pupil diameter (INIT), constriction amplitude (CAMP), and change in diameter (4)). In some cases, the processing unit 106 may further process the video sequences to determine and/or estimate a level of ambient light or ambient illumination. In embodiments, the estimated level of ambient illumination or light can be derived from statistical features extracted from one or more regions of interest in one or more video frames using an ambient light estimator (also referred to as ambient light estimation processor) 210. In some embodiments, the ambient light estimator 210 may comprise a machine learning (ML) light prediction model 212 (e.g., an ML module trained to to extract a level of ambient illumination using one or more input image or video frames). In some cases, a region of interest can be selected by the processing unit 106, e.g., using ambient light estimator 210. In some cases, a score calculator 218 computes a normalized pupil reactivity score that incorporates (directly or indirectly) ambient illumination context and one or more pupillary parameters. In some embodiments, the video frame may be transmitted from the video frame processor 200 to one or more of a pre-flash frame analyzer 202, a flash-onset frame detector 204, and a post-flash frame processor 206. In some embodiments, the processing unit 106 may implement a statistical feature extractor 208 and the post-flash frame processor 206 and extract one of more features (e.g., characteristics of selected regions of a video frame) and provide the extracted features to one or both a pupil parameter measurement engine 214 and the ambient light estimation processor 210. In some embodiments, the light-aware scoring pipeline generates a normalized pupil reactivity score output 230. The normalized score output 230 is provided to a clinical threshold comparator 224, which compares the normalized score output 230 to one or more thresholds and generates one or more alerts and/or categorical classifications output via an alerts/categories module 228 (e.g., reactive vs. unreactive). In some cases, the computing system 106 outputs the normalized score output 230 and any alerts or categories 228 via the user interface 114 and/or transmits such outputs to an external system (e.g., the could server infrastructure 124, the healthcare provider network 128, and/or the electronic medical record system 126).

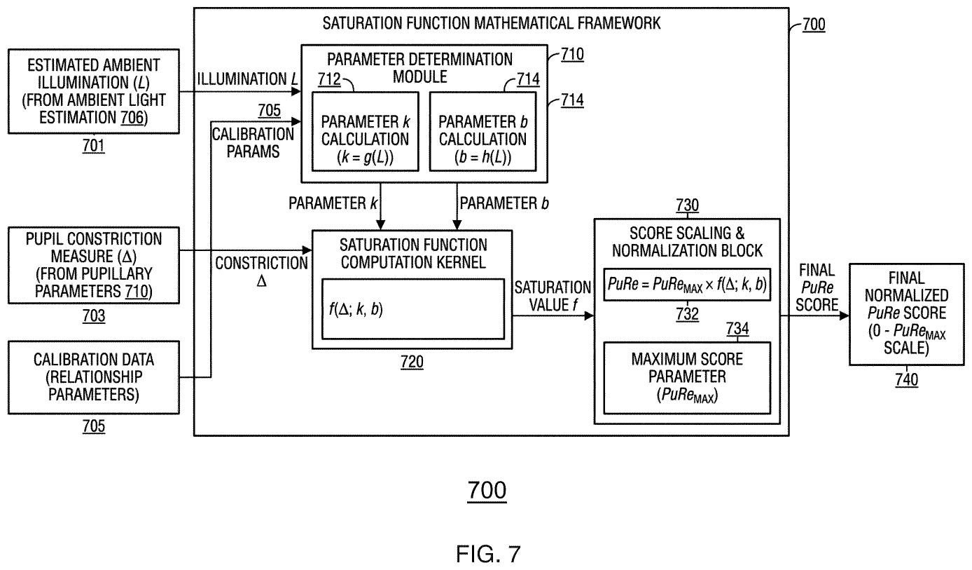

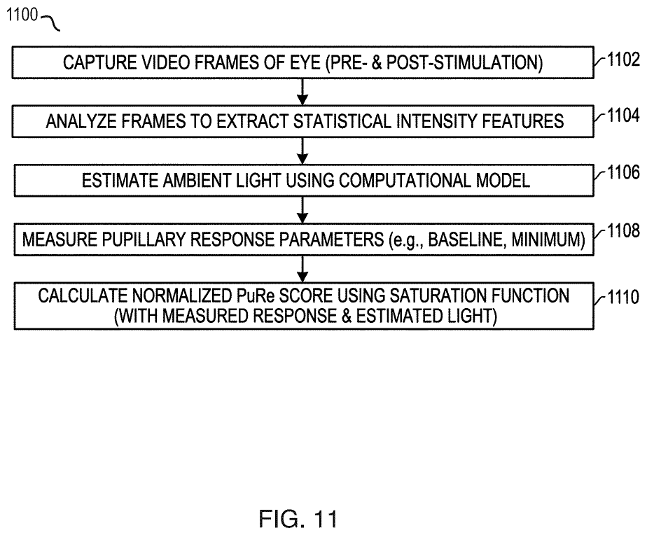

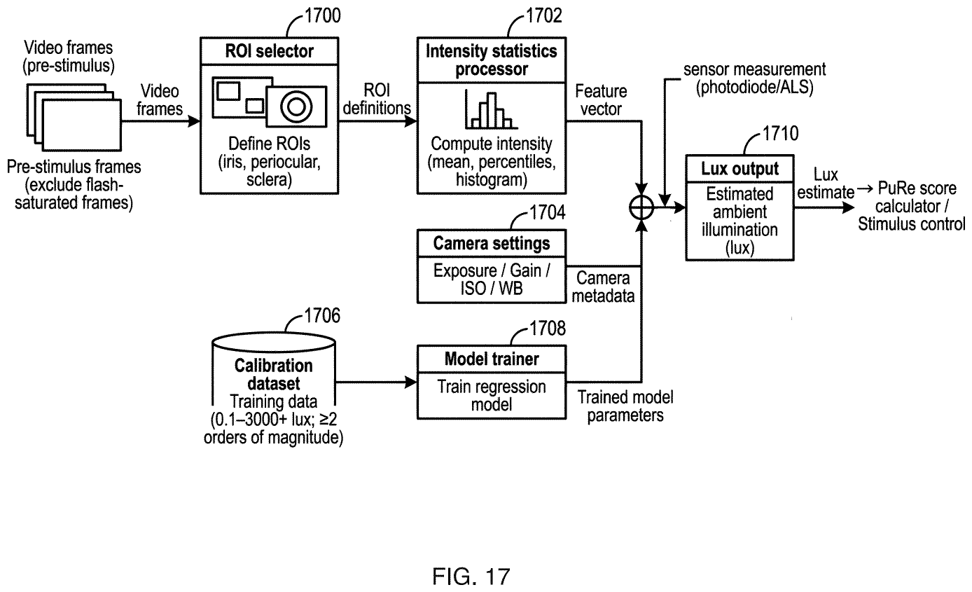

With continued reference to FIG. 2, In some embodiments, the video frame capture processor 200 operates as the foundational element of the light-invariant pupillometry system, establishing the primary data acquisition pathway for the entire computational pipeline. This system addresses the fundamental challenge of ambient light variation in pupillometry, which refers to the quantitative measurement of pupil size and dynamic responses for diagnostic assessment of neurological function and autonomic nervous system activity, by implementing a multi-stage processing architecture that can extract statistical features from video frames captured before, during, and after controlled illumination events. The pipeline can leverage machine learning prediction models and mathematical saturation functions to generate consistent Pupil Reactivity (PuRe) scores, which are standardized measurements that quantify pupillary light reflex responses on a predetermined scale, regardless of environmental lighting conditions, thereby enabling reliable neurological assessment across diverse clinical settings. The term “light-invariant” refers to the system's capability to provide consistent measurements that remain stable and accurate despite variations in ambient lighting conditions, which represents a significant technological advancement over traditional pupillometry methods. Through integration of advanced ambient light estimation, pupil parameter measurement, and quality validation processes, the system can provide clinically actionable measurements that maintain accuracy while compensating for confounding environmental factors. In embodiments, ambient illumination estimation details are described with respect to FIG. 17, a bounded scoring framework is described with respect to FIG. 7, and an example method flow is described with respect to FIG. 11.

In embodiments, as indicated by the arrows in FIG. 2, the video frame capture processor 200 provides a video stream to one or more baseline and timing modules (e.g., a pre-flash frame analyzer 202 and a flash-onset frame detector 204), and the system derives pupillary response parameters and ambient illumination features from the stream. In embodiments, an ambient illumination estimate is produced using an ambient light estimator 210 and/or a machine learning light prediction model 212 and is provided, together with one or more pupillary response parameters, to a saturation function processor 216 and a PuRe score calculator 218. In embodiments, the resulting normalized score output 230 and any classification are output via the user interface 114 and optionally stored and/or exported to an electronic medical record system 126.

In embodiments, a video frame capture processor 200 may comprise one or more camera control modules implemented as executable software instructions executed by a mobile device processor (e.g., the processing unit 106) that may comprise one or more CPU cores. In some cases, a number of the CPU cores in the mobile device or the computing system can be from one to fivr, from five to ten, form ten to twenty, from twenty to thirty-two or larger numbers. In some cases, a CPU core may operate at 1-5 GHz frequencies. In some cases, the video frame capture processor 200 may utilize camera API frameworks such as AVCaptureSession, Camera2 API, or alternative control systems to manage video stream acquisition. In some embodiments, the video frame capture processor 200 may be implemented using dedicated image signal processors, field-programmable gate arrays with 1,000-10,000,000 logic elements, application-specific integrated circuits, system-on-chip solutions, or distributed processing architectures utilizing combinations of CPU cores (1-64), GPU compute units (100-10,000), neural processing units (1-1000 TOPS), or digital signal processors. In embodiments, the video frame capture processor 200 can include frame rate control logic with microsecond to nanosecond precision, circular buffer data structures with capacities ranging from 100 MB to 32 GB, and exposure parameter adjustment utilizing automatic bracketing with real-time histogram analysis. In embodiments, a video acquisition period, may comprise a pre-stimulus period, during which low intensity light is provided to the patient's eye, a stimulus period, during which high intensity light is provided to the patient's eye, and a post-stimulus period, during which the patient's eye is not controllably illuminated but may be exposed to ambient light. Accordingly, the captured video sequence during these periods may comprise a pre-stimulus interval, a stimulus interval, and a post-stimulus interval. In one example, the pre-stimulus interval can include a set of video frames used to establish baseline pupil diameter (INIT) and baseline intensity statistics for ambient illumination estimation, and the post-stimulus interval can include a set of video frames used to quantify constriction and recovery dynamics. In one non-limiting configuration, the video acquisition period and corresponding captured video sequence can have a duration of approximately 5 seconds. In some cases, the captured video sequence can be acquired at approximately 60 frames per second, thereby, in some cases, approximately 300 video frames may be captured during a video acquisition period. In this example, the pre-stimulus period can be approximately 1 second (the pre-stimulus interval can include approximately 60 frames), the stimulus period, during which the light stimulus is delivered, can be approximately 1 second (the stimulus interval can include approximately 60 frames) and the post-stimulus period can be approximately 3 seconds (the post-stimulus interval can include approximately 180 frames), e.g., to capture recovery. However, the durations of one or more periods and number of video frames in one or more intervals can vary; for example, the pre-stimulus interval can be shortened or omitted, the stimulus period can have a duration from approximately 300 milliseconds to approximately 10 seconds, and the post-stimulus period can be shortened or omitted. In embodiments, during each period different stimulus parameters may be used, including different flash intensities. For example, during a pre-stimulus period baseline illumination or a first illumination intensity (e.g., a base flash) may be sued, during the stimulus period interval a pulse and/or a pulse train comprising two or more intensity levels may be used, and during the post-stimulus period a second illumination intensity that differs from the first illumination intensity (baseline illumination intensity). In embodiments, the video frame capture processor 200 can operate at frame rates between 5 Hz and 1000 Hz, preferably between 30 Hz and 120 Hz, most preferably at 60 Hz. In embodiments, the video frame capture processor 200 can utilize alternative sensor configurations, including rolling/global shutter cameras, time-of-flight systems, multi-spectral imaging, and stereo configurations (2-8 cameras), with pixel sizes ranging from 0.5 to 15 μm, resolutions from 480p to 16 K, and dynamic ranges from 60 to 150 dB.

In embodiments, a pre-flash frame analyzer 202 receives the video stream (e.g., comprising the pre-stimulus interval) from the video frame capture processor 200, e.g., during a pre-stimulus period, and performs baseline analysis before controlled stimulation occurs. The pre-flash frame analyzer 202 comprises image processing engines that execute on GPUs (100-10,000 compute units), CPUs (1-128 cores), or NPUs (1-1000 TOPS), utilizing computer vision libraries such as OpenCV, Metal Performance Shaders, CUDA/OpenCL, or proprietary frameworks. In embodiments, the pre-flash frame analyzer 202 can include baseline establishment using moving average filters (3-200 frame windows), weighted averaging, adaptive filtering, pupil detection utilizing Hough circle transform, contour detection, template matching, or machine learning approaches, and environmental parameter extraction through histogram analysis and statistical moment calculations. In embodiments, the pre-flash frame analyzer 202 can process frame sequences over 0.5-30 seconds, establishing stable baseline measurements accounting for natural oscillations. In embodiments, the pre-flash frame analyzer 202 can implement alternative processing approaches, including parallel processing (2-1024 threads), distributed computing, specialized vision processing units (1-64 GB memory), or custom ASIC solutions with communication interfaces including PCIe, DDR memory, Ethernet (10 Mbps-10 Gbps), WiFi 6/7, or 5G/6G cellular.

In embodiments, a flash-onset frame detector 204 operates in parallel with the pre-flash frame analyzer 202, monitoring the video stream to identify the precise moment of controlled illumination initiation. The flash-onset frame detector 204 comprises temporal analysis engines executing on NPU, DSP with dedicated floating-point units, CPU with vector processing, or distributed systems, utilizing signal processing libraries for real-time frame comparison. In embodiments, the flash-onset frame detector 204 can include intensity transition detection using finite difference calculations with adaptive thresholding, cross-correlation functions with template matching, and timing marker generation with 1 μs to 1 ns accuracy through hardware timer interfaces. In embodiments, the flash-onset frame detector 204 can utilize alternative sensor configurations, including photodiodes (1 ns-10 ms response), photomultiplier tubes, avalanche photodiodes (e.g., 100×-1,000,000×gain), photodetectors optimized for UV/visible/NIR wavelengths, or time-correlated single photon counting systems. In embodiments, the flash-onset frame detector 204 can implement processing architectures including FPGA implementations, dedicated DSP chips (1-1000 GMAC/s), or distributed processing with synchronized timing references.

In embodiments, a post-flash frame processor 206 may receives the continuing video stream (e.g., comprising the post-stimulus interval) and timing information from the flash-onset frame detector 204 to analyze the pupillary recovery phase. The post-flash frame processor 206 comprises temporal tracking engines executing on GPU (100-20,000 shader cores), CPU (4-256 cores), or specialized computer vision processors, employing frameworks optimized for dynamic tracking. In embodiments, the post-flash frame processor 206 can include Kalman filtering for motion prediction using state estimation matrices, extended/unscented Kalman filters, particle filtering, temporal curve fitting utilizing exponential decay models with least-squares optimization, and artifact detection using outlier detection with z-score calculations. In embodiments, the post-flash frame processor 206 can implement alternative hardware including multi-core processors with SIMD capabilities (AVX, AVX-512, ARM NEON), dedicated motion estimation units, specialized tracking processors (1-32 GB memory), or distributed computing architectures with communication protocols including shared memory, message passing interfaces, or high-speed interconnects (10 Gbps-1 Tbps).

In embodiments, a statistical feature extractor 208 serves as a central data aggregation point, receiving frame data from the pre-flash frame analyzer 202, flash-onset frame detector 204, and post-flash frame processor 206. The statistical feature extractor 208 computes statistical measures over pixel intensity distributions within one or more regions of interest (ROI).

In embodiments, an ROI can be determined using eye detection results to localize an ocular region and pupil segmentation results, wherein pupil segmentation comprises identifying a pupil boundary, contour, or mask within the ocular region. In some cases, eye detection or eye segmentation may comprise identifying the broader ocular region (e.g., eye opening, iris, sclera, or eyelids), which constrains and contextualizes the pupil segmentation. In some cases, pupil segmentation can be performed based on video frames selected from one or more pre-flash frames to characterize ambient illumination conditions before the controlled stimulus. In embodiments, the statistical feature extractor 208 comprises feature computation engines that execute on a CPU with vectorized SIMD processing, a GPU (100-15,000 compute cores), or specialized mathematical coprocessors, employing libraries such as Intel MKL, NVIDIA cuBLAS, OpenBLAS, or ARM Performance Libraries. In embodiments, the statistical feature extractor 208 can include comprehensive statistical analysis implementing moment calculations (mean, variance, skewness, kurtosis), percentile computations using quickselect approaches or histogram-based methods, and multi-dimensional feature generation employing principal component analysis (PCA) through eigenvalue decomposition. PCA is a dimensionality reduction technique that transforms a set of correlated features into a set of orthogonal components that capture a portion of the variance in the original features. In embodiments, the statistical feature extractor 208 can implement advanced features including higher-order statistical moments, percentile measures (5th-95th percentiles), spatial gradient analysis, windowing techniques (3-100 frame neighborhoods), or dimensionality reduction using PCA, ICA, or t-SNE. In embodiments, the statistical feature extractor 208 can utilize alternative architectures, including parallel computing (2-2000 threads), distributed edge-cloud computing, tensor processing units (1-1000 TOPS), or quantum computing approaches.

In embodiments, an ambient light estimation processor 210 operates as a data fusion center, receiving statistical features from the statistical feature extractor 208 and environmental sensor readings to generate comprehensive ambient light assessments. The ambient light estimation processor 210 comprises sensor fusion engines executing on a multi-core CPU, specialized fusion processors, or distributed architectures, utilizing data fusion libraries that implement Bayesian inference frameworks and calibration systems with polynomial regression models (degrees 1-15). In embodiments, the ambient light estimation processor 210 can include multi-modal data combination using weighted fusion with confidence-based weighting, Bayesian inference, Dempster-Shafer evidence theory, calibration utilizing polynomial regression with device-specific coefficients, and adaptive estimation employing recursive least squares filtering or neural network-based models. In embodiments, the ambient light estimation processor 210 can utilize alternative sensor configurations including photodiodes (280-2500 nm spectral response), photoresistors (1 kΩ-10 MΩ), amplified photodiodes (1×-100,000× gain), colorimeters, lux meters (+1%-+15% accuracy), or specialized light measurement circuits.

In embodiments, a machine learning light prediction model 212 receives feature data from the statistical feature extractor 208 and initial estimates from the ambient light estimation processor 210, applying advanced computational techniques to generate ambient light predictions. The machine learning light prediction model 212 comprises neural network inference engines executing on NPU (1-1000 TOPS), GPU (100-20,000 cores), or specialized AI accelerators, utilizing frameworks such as Core ML, TensorFlow Lite, PyTorch Mobile, ONNX Runtime, or Intel OpenVINO. In embodiments, the machine learning light prediction model 212 can include trained architectures such as random forest classifiers (10-50,000 trees), gradient boosting (50-10,000 rounds), support vector machines with various kernels, or neural networks with 2-100 layers utilizing ReLU, GELU, Swish activation functions and dropout regularization (0.1-0.8 rates), achieving 90%-99.9% prediction accuracy. In embodiments, the machine learning light prediction model 212 can implement alternative AI architectures, including ensemble learning (3-10 model types), federated learning, continual learning, transfer learning, or hybrid classical-quantum approaches with hardware acceleration, including neuromorphic chips (100-100,000 neurons), photonic processors, or specialized inference engines.

In embodiments, a pupil parameter measurement engine 214 receives video data from the post-flash frame processor 206, timing information from the flash-onset frame detector 204, and baseline measurements from the pre-flash frame analyzer 202 to perform comprehensive quantification of pupillary responses. The pupil parameter measurement engine 214 comprises computer vision analysis engines that execute on GPUs with specialized tensor processing units, CPUs with vector processing, or distributed processing systems, utilizing deep learning frameworks optimized for real-time inference. In embodiments, the pupil parameter measurement engine 214 can include pupil segmentation implementing convolutional neural network architectures (U-Net, Mask R-CNN, DeepLab, custom architectures with 10-500 layers), boundary detection utilizing edge detection methods (Canny with 50-300 thresholds, Sobel, Laplacian) with sub-pixel accuracy through ellipse fitting, and temporal parameter extraction employing velocity calculations with numerical differentiation and smoothing filters. In embodiments, the pupil parameter measurement engine 214 can implement alternative approaches including parallel processing (4-2048 threads), distributed computing, specialized vision processing units (1-128 GB memory), custom ASIC solutions, or sensor configurations including stereo cameras, depth cameras (1 mm-10 cm accuracy), structured light systems, or time-of-flight sensors (0.1-10 m range).

In embodiments, a saturation function processor 216 serves as the computational core for generating light-invariant measurements, receiving pupillary parameters from the pupil parameter measurement engine 214, and ambient light data from the machine learning light prediction model 212. The saturation function processor 216 comprises mathematical computation engines that execute on a CPU with dedicated floating-point units, a GPU with compute capabilities, or specialized mathematical coprocessors with 16-128 bit precision, utilizing mathematical libraries for transcendental function calculations. In embodiments, the saturation function processor 216 can include exponential saturation function implementation utilizing f(x)=1-e\hat{ }(−kx\hat{ }b), sigmoid functions, hyperbolic tangent functions, power law functions, or piecewise linear functions with parameter optimization using gradient descent (learning rates 0.001-1.0), Newton-Raphson methods, genetic approaches, light-dependent parameter adjustment using interpolation techniques (linear, spline degrees 1-10, polynomial) or lookup table methods (100-100,000 entries), and normalization employing linear scaling, logarithmic scaling, z-score normalization, or min-max scaling. In embodiments, the saturation function processor 216 can implement alternative approaches, including approximation techniques, table-based function evaluation, polynomial approximations (degrees 3-50), vector processors for SIMD operations (e.g., 128-bit to 1024-bit widths), or distributed mathematical computing.

In embodiments, a Light-Dependent Parameter Adjuster 702 receives (i) an estimated ambient illumination level and (ii) normative data 706 encoding illumination-dependent reference relationships. The Light-Dependent Parameter Adjuster 702 processes the ambient illumination input by selecting, interpolating, or computing one or more illumination-conditioned parameters for a saturation function (e.g., rate and/or shape parameters) using the normative data 706. The Light-Dependent Parameter Adjuster 702 outputs the illumination-conditioned parameters to the saturation function processor 216 for use in generating standardized measurements.

The saturation function processor 216 combines the pupillary response parameters received from the pupil parameter measurement engine 214, one or more statistical intensity features received from the statistical feature extractor 208, and illumination-conditioned parameters supplied by the Light-Dependent Parameter Adjuster 702 to apply a saturation-type mapping that compensates for ambient illumination effects and produces standardized measurements. The PuRe score calculator 218 receives the standardized measurements from the saturation function processor 216 and scales and normalizes the measurements to generate the normalized pupil reactivity score output 230.

In embodiments, the saturation function processor 216 receives (i) pupillary response parameters from the pupil parameter measurement engine 214 and (ii) ambient illumination information, and applies a light-dependent saturation mapping using illumination-conditioned parameters to generate standardized, light-compensated measurement values for downstream scoring.

In embodiments, a Light-Dependent Parameter Adjuster 702 receives an estimated ambient illumination level and normative data 706. The Light-Dependent Parameter Adjuster 702 processes the ambient illumination level by selecting, interpolating, or computing illumination-conditioned parameters for a saturation function using the normative data 706. The Light-Dependent Parameter Adjuster 702 outputs the illumination-conditioned parameters to the saturation function processor 216 for use in generating standardized measurements.

Accordingly, one or more saturation function parameters used to compute the normalized pupil reactivity score are selected or calculated based on the determined ambient illumination level.

In embodiments, a PuRe score calculator 218 receives standardized measurements from the saturation function processor 216 and integrates them with light compensation data to generate the final standardized pupil reactivity score. The PuRe score calculator 218 comprises score computation engines that execute on CPUs with specialized arithmetic logic units, utilizing mathematical computation libraries optimized for high-precision numerical processing. In embodiments, the PuRe score calculator 218 can include standardized scoring implementing PuRe=PuReMAX×(1−e^{-kΔ}^{^{b}}), where PuReMAX represents a configurable maximum value of a PuRe scoring range, and k and b represent parameters selected based on calibration data and can be dependent on ambient illumination. In embodiments, Δ denotes a constriction metric. In some embodiments, Δ is a relative constriction defined as Δ=CAMP/INIT (optionally expressed as a percent), where CAMP=INIT-MIN, INIT denotes a baseline pupil diameter prior to a stimulus, and MIN denotes a minimum pupil diameter after the stimulus. In other embodiments, Δ is defined as an absolute constriction amplitude (e.g., CAMP) and/or a robust constriction depth estimate derived from a smoothed pupillogram while excluding blink frames.

In embodiments, the PuRe score calculator 218 scales and normalizes the standardized measurements received from the saturation function processor 216 to produce the normalized pupil reactivity score 230 on a defined scoring range.

In embodiments, the PuRe score calculator 218 computes PuRe using a bounded mapping that depends on (i) one or more pupillometry-derived response parameters and/or a pupillogram time-series summary and (ii) one or more context variables representing illumination and/or confounding conditions. In some embodiments, the processing unit forms: (a) a response feature vector x and (b) a context vector c. Non-limiting examples of x include Δ, CAMP, latency, constriction/dilation velocities, recovery time, and/or a time-series summary. Non-limiting examples of c include an estimated ambient illumination metric \hat{L} (e.g., lux or log₁₀(lux)), INIT used as an illumination proxy, camera exposure and gain parameters, stimulus intensity settings, and quality metrics.

In embodiments, PuRe is computed as:

$$Re = Post ( PuReMAX \cdot F ( x ; θ ( c ) ) ) .$$

Where F(·) is a bounded, monotone saturation mapping and θ(c) denotes parameters selected based on context c. In embodiments, Post(·) applies one or more post-processing operations including clipping to a bounded range (e.g., [0, PuReMAX]), rounding/quantization to a clinical increment, mapping to a minimum nonzero value when a response is detected, and/or applying quality-control overrides that output a quality flag and/or suppress output values when quality conditions are met.

In embodiments, F(·) is selected from one or more curve families, including: (i) an exponential family with an optional nonzero lower asymptote L, where F(Δ; k, b, L)=L+(1−L)·(1−exp (−kΔ^{b})); (ii) a logistic/Hill family operating on a multi-parameter response magnitude M, where F (M; d₀, w, L)=L+(1−L). (1+exp (−(M−d₀)/w))−1; (iii) a lookup table surface over (Δ, INIT) and/or (M, Î) with interpolation and optional monotonic constraints; and/or (iv) a hybrid model combining a parametric mapping with a learned correction trained to reduce residual dependence on illumination and/or confounders. In embodiments, k, b, L, d₀, and/or w are obtained by fitting the mapping to normative datasets across multiple illumination levels and/or by storing parameters in one or more lookup tables indexed by ambient illumination and/or baseline diameter INIT.

In embodiments, the processing unit 106 implements a light-dependent, double-threshold exponential mapping in which an ambient illumination metric L₁₀=log₁₀(lux) (or another monotone transform of illumination) is used to select one or more constriction thresholds. In embodiments, L₁₀ is clipped to an operating range (e.g., about −1 to about 4). In embodiments, the processing unit computes a light-dependent abnormal threshold Δ_{abn}(L₁₀) and a light-dependent healthy threshold Δ_{healthy}(L₁₀) by interpolation between reference thresholds defined at a dim reference illumination and a bright reference illumination. In embodiments, the thresholds are expressed as a constriction percentage and span from single-digit percent values in bright conditions to tens of percent in dim conditions. In embodiments, the processing unit selects parameters k>0 and P₀ such that PuRe (Δ_{abn})=PuRe₁ and PuRe(Δ_{healthy})=PuRe₂, where PuRe₁ and PuRe₂ are target scores within [0, PuReMAX]. The processing unit then computes an exponential mapping, for example:

$$Re = Clip ( U - ( U - {P}_{0} ) exp ( - k Δ ) ) ,$$

where U=PuReMAX and Clip(·) bounds the output to a defined range. In embodiments, when Δ=0 the processing unit overrides the mapping and outputs PuRe=0 to indicate no detected constriction.

In embodiments, the processing unit 106 implements an init-dependent sigmoid mapping in which baseline diameter INIT is used as a proxy for illumination context when a light sensor is unavailable or unreliable. In embodiments, the processing unit computes a context-dependent midpoint 40 (INIT) using a sigmoid function:

$${Δ}_{0} ( INIT ) = {Δ}_{min} + \frac{{Δ}_{max} - {Δ}_{min}}{1 + exp ( - s ( INIT - {INIT}_{0} ) )} ,$$

where Δₘᵢₙ is a lower asymptote (which can be below zero or near zero), Δₘₐₓ is an upper asymptote (e.g., about 10% to about 40%), s is a steepness parameter, and INIT₀ is a midpoint within a typical pupil diameter range. The processing unit then computes a bounded sigmoid mapping, for example:

$$Re = \frac{U}{1 + exp ( - k ( Δ - {Δ}_{0} ( INIT ) ) )} ,$$

where U=PuReMAX. In embodiments, the processing unit applies quantization and minimum-nonzero coercion as described above. In embodiments, the processing unit additionally applies a fail-safe quality check in very bright conditions (e.g., when L₁₀ exceeds a configured threshold) by outputting a quality flag and suppressing an output score below a healthy threshold, thereby prompting user guidance and/or recapture.

In embodiments, confidence metrics are calculated using bootstrap sampling or Bayesian estimation. In embodiments, the PuRe score calculator 218 can utilize alternative architectures, including ensemble scoring, adaptive scoring that adjusts to patient populations, personalized scoring using individual baselines (days to years of historical data), or dedicated scoring engines (1 GFLOPS to 100 TFLOPS), parallel processing, or specialized arithmetic units.

In embodiments, a light compensation controller 220 receives ambient light estimates from both the ambient light estimation processor 210 and the machine learning light prediction model 212, managing dynamic system adjustments based on real-time environmental conditions. The light compensation controller 220 comprises control system engines that execute on a CPU with real-time scheduling, specialized control processors, or dedicated control hardware, utilizing control systems libraries that implement feedback control frameworks. In embodiments, the light compensation controller 220 can include adaptive control implementing PID control with proportional, integral, and derivative gains, and predictive control utilizing model predictive control with one or more prediction horizons, state estimation using Kalman filtering or observer-based approaches, and parameter adjustment employing gain scheduling with environmental condition mapping through multi-dimensional lookup tables. In embodiments, the light compensation controller 220 can implement alternative architectures including distributed control across multiple nodes, hierarchical control (2-10 levels), event-driven control, fuzzy logic control, neural network-based control, or quantum control approaches with communication interfaces including real-time Ethernet (<1 ms latency), CAN bus (125 kbps-5 Mbps), I2C, SPI (1-100 MHZ), or wireless protocols.

In embodiments, a normalized output generator 222 receives the normalized score output 230 from the PuRe score calculator 218 and formats the normalized score output 230 for clinical integration and compatibility with healthcare systems. In embodiments, the normalized score output 230 produced by the PuRe score calculator 218 is distributed (as copies) to the normalized output generator 222, the clinical threshold comparator 224, the uncertainty quantifier 722, and the confidence interval generator 724. The normalized score output generator 222 comprises data formatting engines executing on CPUs with dedicated data processing capabilities, utilizing data serialization libraries that implement standardized healthcare data formats, including HL7 FHIR compliance frameworks. In embodiments, the normalized score output generator 222 can include report generation implementing template-based formatting with healthcare data structures (XML, JSON serialization, HL7 v2.x-3.x, DICOM), data validation utilizing schema checking with comprehensive error handling, and communication protocols implementing secure transmission with end-to-end encryption (AES-128 to AES-256, TLS 1.2-1.3) and authentication mechanisms. In embodiments, the normalized score output generator 222 can implement alternative architectures, including microservices-based formatting, containerized processing (e.g., Docker, Kubernetes), serverless functions (with execution times ranging from 1 ms to 15 minutes), edge-to-cloud synchronization, or blockchain-based integrity verification with communication protocols (e.g., RESTful APIs, GraphQL, gRPC, with data transfer rates ranging from 1 Mbps to 10 Gbps, and message queues).

In embodiments, a clinical threshold comparator 224 receives the normalized score output 230 produced by the PuRe score calculator 218 (e.g., via the normalized output generator 222) and evaluates the normalized score output 230 against established clinical thresholds to provide immediate diagnostic insights. The clinical threshold comparator 224 comprises decision support engines executing on a CPU with specialized comparison logic units, distributed decision systems, or dedicated decision processing hardware, utilizing decision logic libraries implementing rule-based inference engines. In embodiments, the clinical threshold comparator 224 can include threshold comparison implementing evidence-based comparison logic with statistical significance testing (t-tests, Mann-Whitney U, chi-square, ANOVA), multi-threshold systems supporting 3-50 threshold levels, adaptive threshold approaches adjusting based on patient demographics, and alert generation utilizing priority-based notification systems with escalation protocols (immediate alerts<1 second response, delayed notifications 1 second-24 hours) and validation request methods employing cross-referencing with quality assurance protocols. In embodiments, the clinical threshold comparator 224 can implement alternative architectures including machine learning-based decision systems (support vector machines, random forests, neural networks), expert systems with rule-based inference (10-10,000 rules), fuzzy logic systems, ensemble decision-making, or quantum computing with alerting mechanisms (push notifications, SMS, email, hospital communication systems, voice alerts, wearable device integration).

In embodiments, a quality validation engine 226 operates as the final verification element, receiving data inputs from multiple processing stages, including the flash-onset frame detector 204, light compensation controller 220, and clinical threshold comparator 224. The quality validation engine 226 comprises multi-parameter validation systems that execute across CPU, GPU, and NPU resources, utilizing distributed processing capabilities. These systems employ comprehensive validation frameworks that incorporate statistical analysis libraries and machine learning-based quality assessment models. In embodiments, the quality validation engine 226 can include quality metric computation implementing signal-to-noise ratio calculations with frequency domain analysis (Fast Fourier Transform, wavelet analysis, spectral analysis 0.1-1000 Hz), measurement consistency utilizing correlation analysis (Pearson/Spearman correlation thresholds 0.5-0.99) and regression techniques (linear, polynomial degrees 1-20, machine learning regression), and artifact detection employing anomaly detection based on statistical process control methods and machine learning techniques (isolation forests, one-class SVM, autoencoders, ensemble detection). In embodiments, the quality validation engine 226 can utilize alternative architectures including distributed validation across multiple nodes, real-time validation with streaming data processing (1 MB/s-10 GB/s), batch validation (1 minute-24 hour windows), cloud-based validation with edge computing, or blockchain-based validation with quality metrics including image quality assessments (0-100 scores), temporal consistency measures, cross-device validation, population-based scoring (1000-1,000,000 subject databases), or adaptive quality thresholds.

The light-invariant processing pipeline represents a significant technological advancement over conventional pupillometry approaches through a multi-stage computational architecture combining statistical feature extraction, machine learning-based ambient light prediction utilizing neural network models (1000-10,000,000 parameters), and mathematical saturation function processing employing exponential transformation methods generating consistent measurements across environmental lighting conditions (e.g., about 0.1-3000 lux, and in some embodiments up to about 10,000 lux). The system achieves unprecedented measurement accuracy of ±0.025 mm or better, surpassing some of the existing infrared pupillometers by factors of 2 to 10 through advanced computer vision techniques that utilize convolutional neural networks (10 to 100 layers) and multi-frame analysis procedures. Real-time processing capabilities, implemented through distributed computing across CPU cores (4-256), GPU compute units (100-20,000), and NPU processing elements (1-1000 TOPS), enable immediate clinical feedback while maintaining computational efficiency. The comprehensive quality validation framework ensures clinical-grade reliability through multi-parameter assessment techniques evaluating signal quality using 10-200 individual statistical metrics, measurement consistency through correlation analysis and regression methods, and artifact detection using statistical process control and machine learning approaches. These technical advances collectively enable deployment of accurate, reliable pupillometry assessment using ubiquitous mobile computing devices with alternative hardware configurations (smartphones, tablets, dedicated medical devices, wearable systems, distributed sensing architectures), democratizing access to quantitative neurological evaluation while providing multiple technological implementation pathways for enhanced clinical effectiveness, operational reliability, and competitive differentiation.

In embodiments, FIG. 17 illustrates estimation of ambient illumination using one or more of sensor-based measurements and image-based computational estimation. In embodiments, a region of interest selector 1700 identifies one or more ROIs in one or more frames, an intensity statistics processor 1702 computes statistical features over pixel intensities, and one or more camera settings 1704 (e.g., exposure or gain) are optionally used as additional inputs. In embodiments, a calibration dataset 1706 is collected across multiple illumination levels spanning at least two orders of magnitude and is used to train or fit a model using a model trainer 1708, and the model outputs a lux estimate 1710 used by downstream scoring. In embodiments, estimating ambient illumination comprises compensating for stimulus illumination using one or more pre-stimulus frames and/or excluding one or more frames affected by the light stimulus. In embodiments, estimating ambient illumination from video frames comprises computing one or more statistics (e.g., mean, median, trimmed mean, variance, percentiles, histogram features, saturated pixel fraction, and/or per-channel features) over one or more ROIs comprising one or more of sclera, iris, eyelid, periocular skin, and/or background regions, and optionally using raw or linearized pixel values (e.g., before tone mapping) when available. In embodiments, estimating ambient illumination further comprises using one or more frames spanning a controlled-illumination transition (e.g., a flash-onset or flash-offset transition) to infer ambient illumination and/or stimulus contribution, for example, by analyzing a pair of transition frames, including a frame immediately before and immediately after flash onset or offset, to compute an intensity change (e.g., via RMS or finite-difference measures) prior to auto-exposure compensation. Single-frame estimation may be used as an alternative when combined with calibrated camera metadata. In embodiments, such transition-based estimation supports compensation for multi-level illumination protocols (e.g., a base illumination level, a stimulus pulse and/or pulse train, and a post-stimulus illumination level that differs from the base illumination level). In embodiments, camera settings 1704 comprise one or more of exposure time, analog gain, digital gain, ISO, white balance gains, aperture, focus distance, frame rate, and/or HDR mode, and such settings are used to normalize or interpret intensity statistics for ambient illumination estimation and/or to detect saturation or under-exposure conditions. In embodiments, sensor-based measurements comprise a photodiode, photoresistor, ambient light sensor, or other light sensor integrated with the device, the camera module, and/or an eye cup, and the sensor measurement is used alone or fused with image-based estimation (e.g., weighted averaging, Kalman filtering, or model-based fusion). In embodiments, baseline pupil diameter (INIT) is used as an auxiliary proxy for ambient illumination (e.g., via a learned mapping conditioned on age and/or acquisition geometry) and/or as a fallback when sensor readings or image-based estimators are unavailable, unreliable, or affected by stimulus illumination. In embodiments, the ambient illumination estimate of FIG. 17 is used by the scoring pipeline of FIG. 2 and by the end-to-end operation of FIG. 18.

In embodiments, as indicated by the arrows in FIG. 17, the region of interest selector 1700 provides one or more ROI definitions to the intensity statistics processor 1702, which generates feature values that are combined with camera settings 1704 and/or sensor measurements. In embodiments, the model trainer 1708 uses the calibration dataset 1706 to generate one or more calibrated models, and the calibrated model outputs a lux estimate 1710 that is provided to downstream scoring and/or stimulus control to support substantially light-invariant outputs.

FIG. 3 is a diagram of a multi-frame integration architecture system in accordance with embodiments of the present disclosure. In some embodiments, the multi-frame integration architecture system, may comprise a sequential video frame buffer 300 configure to receive and store a plurality of video frames from the imaging device 102. In some cases, the sequential video frame buffer 300 may comprise a dedicated high-speed memory circuit configured with a ring buffer architecture and multi-port access controllers to temporarily store a continuous sequence of video frames captured by the imaging device 102. In embodiments, as indicated by the arrows in FIG. 3, the video frames may be transmitted from the sequential video frame buffer 300 to one or both a sub-pixel motion detector 302 and a parallax analysis model. In some cases, the output of the sub-pixel motion detector 302 may be provided to a frame alignment system 304, and then in parallel to a multi-frame super-resolution processor 306 and a temporal averaging engine 308 configured to identify and suppress random noise variations. In embodiments, enhanced frame data generated by the multi-frame super-resolution processor 306 and a temporal averaging engine 308 may be provided to an enhanced image reconstructor 316. In some cases, the output of the enhanced image reconstructor 316 may be transmitted to a pupil boundary detector 318 configured to generate boundary coordinate data (e.g., pupil boundary coordinate data). In some cases, the enhanced image reconstructor 316 may receive noise reduction data from a noise reduction filter 314 and further enhance an image based on the noise-reduction data. In some cases, the output of the pupil boundary detector 318 may be transmitted to a high-precision measurement extractor 320 configured extract specified parameters extraction from an enhanced image generated by the enhanced image reconstructor 316 based on boundary coordinate data generated by the pupil boundary detector. In some cases, the output of the high-precision measurement extractor 320 may be transmitted to a quality enhancement validator 324 configured to validate the output of the high-precision measurement extractor 320 prior to downstream pupillometry parameter extraction and scoring. In some embodiments, the multi-frame integration architecture system shown in FIG. 3, may comprise a confidence score generator 322 configured to generate a confidence score based at least in part on the noise-reduced data received from the temporal averaging engine 308 and provide the confidence score to the quality enhancement validator 324 that may output validated enhanced measurements and the confidence score based at least in part on data received from the high-precision measurement extractor 320 and the quality enhancement validator 324. In some cases, the parallax analysis module 310 may use the video frames received from the sequential video frame buffer 300 to generate depth classification data and transmit the depth classification data to the artifact identification system 312. In some cases, the artifact identification system 312 may generate artifact classification data and provide the artifact classification data to the enhanced image reconstructor 316, noise reduction filter 314, and a confidence score generator 322. As such in some cases, the confidence score generator 322 may generate the confidence score based on one or both the artifact classification data received from the artifact identification system 312 and the noise-reduced data received from the temporal averaging engine 308.

In embodiments, the sequential video frame buffer 300 receives raw video frames and outputs ordered frame sequences; the sub-pixel motion detector 302 processes the frame sequence to output motion vectors; the frame alignment system 304 uses the motion vectors to output geometrically corrected frame data; the multi-frame super-resolution processor 306 and temporal averaging engine 308 process aligned frames to output enhanced and noise-reduced image data; the enhanced image reconstructor 316 outputs reconstructed images; the pupil boundary detector 318 outputs pupil boundary coordinates; and the high-precision measurement extractor 320 outputs pupillary measurement parameters with associated uncertainty for downstream scoring. In embodiments, the validated measurements and enhanced imagery produced by the multi-frame integration architecture of FIG. 3 are provided to the pupillary parameter measurement engine 214 and statistical feature extractor 208 of FIG. 2 to improve accuracy of pupillary response parameter extraction and subsequent score calculation.

In some embodiments, the multi-frame integration architecture may be used (e.g., primarily) for lower-level measurement of pupil diameter versus time. It operates independently of the quality scoring engine. Outputs of multi-frame integration may optionally contribute inputs to quality assessment but are not sufficient on their own to generate a quality score or normalized reactivity score

In embodiments, the sequential video frame buffer 300 can include DRAM memory banks with specialized memory controllers that implement circular addressing logic and frame indexing circuits to maintain temporal ordering of incoming frame data. The sequential video frame buffer 300 incorporates DMA transfer circuits and parallel data pathways that provide rapid access to both current and previously captured frames for subsequent processing operations. In embodiments, the sequential video frame buffer 300 can utilize cache coherency circuits and memory management units that efficiently handle continuous data streams without memory overflow conditions through hardware-based pointer management and automatic garbage collection circuits. The sequential video frame buffer 300 transmits frame data through high-bandwidth data buses to the sub-pixel motion detector 302 while providing (e.g., substantially simultaneously) parallel access to frame sequences for the frame alignment system 304.

In embodiments, the sub-pixel motion detector 302 comprises dedicated correlation processing circuits and optical flow computation hardware that may operate in direct communication with the sequential video frame buffer 300 through dedicated data interface circuits. In embodiments, the sub-pixel motion detector 302 can include specialized digital signal processing (DSP) cores and parallel correlation engines that analyze pixel intensity variations across sequential frames using phase correlation hardware and interpolation processing circuits. In some implementations, the sub-pixel motion detector 302 may employ dedicated arithmetic logic units (ALUs) and floating-point processing circuits combined with lookup table memory to detect, estimate, and/or determine relative motion between an imaged object (e.g., eye of a patient) and the camera, resulting in the motion of the image scene in the camera video frame, between camera frames, in some cases, with sub-pixel accuracy using hardware-accelerated mathematical operations. In embodiments, the sub-pixel motion detector 302 can incorporate vector processing units and SIMD (Single Instruction, Multiple Data) circuits that detect motion displacements through parallel computation hardware. The sub-pixel motion detector 302 generates motion vector data through dedicated output buffer circuits that feed directly into input staging circuits of the frame alignment system 304 via dedicated signal pathways.

In embodiments, the frame alignment system 304 may comprise a geometric transformation processing circuit and dedicated matrix multiplication hardware that receives motion vector data from the sub-pixel motion detector 302 through dedicated input buffer circuits. In embodiments, the frame alignment system 304 can include specialized coordinate transformation engines and affine processing circuits that apply geometric registration operations across the frame sequence using dedicated transformation matrix computation hardware. The frame alignment system 304 utilizes dedicated interpolation circuits and resampling hardware combined with coordinate mapping circuits to compensate for detected motion patterns through real-time geometric correction processing. In embodiments, the frame alignment system 304 can incorporate dedicated warping engines and perspective correction circuits that achieve registration accuracy through specialized geometric processing units. The frame alignment system 304 may produce geometrically corrected frame data through output formatting circuits that feed the resulting geometrically corrected frame data to one or both the multi-frame super-resolution processor 306 and temporal averaging engine 308, e.g., through dedicated parallel data distribution circuits.

In embodiments, the multi-frame super-resolution processor 306 may comprise a dedicated reconstruction processing circuit and frequency domain computation hardware that operates on aligned frame sequences received from the frame alignment system 304 through dedicated high-speed data interfaces. In embodiments, the multi-frame super-resolution processor 306 can include specialized Fourier transform processing units and upsampling circuits that combine information from multiple aligned frames, e.g., output by the frame alignment system 304, using a dedicated fusion algorithm implemented in hardware acceleration circuits. In some implementations, the multi-frame super-resolution processor 306 implements an iterative reconstruction circuit and deconvolution processing hardware that can exploit sub-pixel sampling differences through a dedicated mathematical processing engine and/or a convolution acceleration circuit. In embodiments, the multi-frame super-resolution processor 306 can incorporate dedicated an edge enhancement circuit and detail reconstruction hardware that achieve resolution enhancement through specialized image processing pipelines implemented in configurable logic circuits. In some embodiments, the multi-frame super-resolution processor 306 may generate enhanced resolution data, and transmit the enhanced resolution data, e.g., through a dedicated output buffer circuit, to the enhanced image reconstructor 316. In some such embodiments, the multi-frame super-resolution processor 306 may providing data (e.g., enhanced resolution data) to the artifact identification system 312 through a data pathway (not shown) independent and parallel to a data pathway established between the multi-frame super-resolution processor 306 and the enhanced image reconstructor 316.

In embodiments, the temporal averaging engine 308 may comprise a statistical processing circuit and noise reduction hardware configured to processes aligned frame sequences received from the frame alignment system 304 through dedicated input staging circuits. In embodiments, the temporal averaging engine 308 can include a dedicated accumulation circuit and weighted averaging hardware configured to apply statistical operations across temporal sequences using specialized arithmetic processing units and variance computation circuits. In some cases, the temporal averaging engine 308 may utilize a dedicated outlier detection circuit and quality weighting hardware combined with a temporal consistency analysis circuit to identify and suppress random noise variations through hardware-implemented statistical algorithms. In embodiments, the temporal averaging engine 308 can incorporate dedicated filtering circuits and noise suppression hardware that process frame sequences through configurable processing pipelines implemented in specialized signal processing circuits. In some cases, the temporal averaging engine 308 may generate noise-reduced data and transmit the noise-reduced data, e.g., through a dedicated output formatting circuit, to the enhanced image reconstructor 316. In some cases, the temporal averaging engine 308 may provide a temporal consistency metric to the quality enhancement validator 324, e.g., through a dedicated measurement pathway. In embodiments, the random noise variations comprise frame-to-frame intensity fluctuations and measurement noise arising from sensor noise and illumination variability.

In embodiments, the parallax analysis 310 may comprise a depth discrimination processing circuit and motion analysis hardware configured tot examines differential motion characteristics using dedicated computational engines connected to the frame alignment system 304 through specialized data interface circuits. In embodiments, the parallax analysis 310 can include dedicated stereo processing circuits and depth estimation hardware that can distinguish between consistent anatomical structures and transient artifacts using specialized feature tracking circuits and motion vector analysis engines. In some cases, the parallax analysis 310 may leverage a dedicated viewpoint processing circuit and perspective analysis hardware to create multiple viewpoints through specialized geometric computation engines and coordinate transformation circuits. In embodiments, the parallax analysis 310 can incorporate dedicated depth mapping circuits and feature classification hardware that detect depth differences through specialized range computation processors and spatial analysis circuits. In some embodiments, the parallax analysis module 310 may transmit depth classification data, e.g., through a dedicated output buffer circuit, to the artifact identification system 312. In some cases, the parallax analysis module may provide motion analysis results to the enhanced image reconstructor 316 through a signal routing circuit independent of a data pathway through which the depth classification data is transmitted to the artifact identification system 312.

In embodiments, the artifact identification system 312 may comprise a pattern recognition processing circuit and classification hardware configured to utilize depth map data received from the parallax analysis 310 and resolution-enhanced data received from the multi-frame super-resolution processor 306, e.g., through a dedicated multi-input interface circuit. In embodiments, the artifact identification system 312 can include dedicated machine learning acceleration circuits and neural network processing hardware that apply classification algorithms using specialized inference engines and feature extraction circuits. The artifact identification system 312 may analyze temporal consistency data through dedicated sequence processing circuits and geometric analysis hardware combined with intensity characteristic evaluation circuits to identify unwanted image elements through real-time classification processing. In embodiments, the artifact identification system 312 can incorporate dedicated segmentation circuits and boundary detection hardware that detect artifacts through specialized contour analysis processors and region classification engines. In some cases, the artifact identification system 312 can produce artifact classification data, and transmit the artifact classification data to the noise reduction filter 314, e.g., through a dedicated output formatting circuit. In some cases, the artifact identification system 312 may provide artifact location information to the enhanced image reconstructor 316 through a data distribution circuit independent of a data pathway established between the artifact identification system 312 and the noise reduction filter 314.

In embodiments, the noise reduction filter 314 may comprise an adaptive filtering circuits and signal processing hardware configured to operate on processed frame sequences while receiving artifact identification data from the artifact identification system 312 through dedicated control interface circuits. In embodiments, the noise reduction filter 314 can include dedicated adaptive algorithm circuits and filtering coefficient computation hardware that adjust filtering characteristics using specialized parameter adaptation engines and response optimization circuits. The noise reduction filter 314 applies spatiotemporal filtering operations through dedicated convolution processing circuits and frequency domain filtering hardware that exploit both spatial and temporal correlation through parallel processing engines. In embodiments, the noise reduction filter 314 can incorporate dedicated noise estimation circuits and suppression control hardware that achieve noise reduction through specialized signal enhancement processors and quality optimization circuits. The noise reduction filter 314 generates cleaned signal data through dedicated output buffer circuits that feed the enhanced image reconstructor 316 while providing noise reduction metrics to the quality enhancement validator 324 through dedicated measurement interface circuits.

In embodiments, the enhanced image reconstructor 316 may comprise multi-input fusion processing circuits and image synthesis hardware configured to combine data streams from the multi-frame super-resolution processor 306, temporal averaging engine 308, parallax analysis 310, and noise reduction filter 314 through dedicated multi-channel input interface circuits. In embodiments, the enhanced image reconstructor 316 can include dedicated blending circuits and quality-weighted fusion hardware that preserve benefits from each processing stage using specialized combination algorithms implemented in dedicated processing engines. The enhanced image reconstructor 316 implements quality-aware processing circuits and adaptive blending hardware that weight contributions from different processing paths through dedicated quality assessment circuits and weighting computation engines. In embodiments, the enhanced image reconstructor 316 can incorporate dedicated optimization circuits and image enhancement hardware that produce final processed imagery through specialized reconstruction pipelines implemented in configurable processing circuits. The enhanced image reconstructor 316 transmits optimized image data through dedicated high-speed data pathways to the pupil boundary detector 318 while simultaneously providing quality metrics to the confidence score generator 322 through independent measurement circuits.

In embodiments, a pupil boundary detector 318 comprises edge detection processing circuits and boundary analysis hardware that analyzes enhanced imagery received from the enhanced image reconstructor 316 through dedicated input buffer circuits. In embodiments, the pupil boundary detector 318 can include dedicated gradient computation circuits and contour detection hardware that identify pupillary edges using specialized edge processing engines and elliptical fitting acceleration circuits. The pupil boundary detector 318 utilizes dedicated shape analysis circuits and geometric fitting hardware combined with sub-pixel precision processing circuits to achieve accurate boundary localization through real-time contour analysis engines. In embodiments, the pupil boundary detector 318 can incorporate dedicated validation circuits and boundary refinement hardware that ensure detection accuracy through specialized verification processors and consistency checking circuits. The pupil boundary detector 318 generates precise boundary coordinate data through dedicated output formatting circuits that feed the high-precision measurement extractor 320 while providing boundary quality metrics to the confidence score generator 322 through dedicated measurement interface pathways.

In embodiments, the high-precision measurement extractor 320 may comprise a geometric computation circuit and precision measurement hardware configured to use detected boundary coordinates received from the pupil boundary detector 318, e.g., through a dedicated input staging circuit, to compute a geometrical parameter of the patient's eye 122 (e.g., a pupil related geometrical parameter). In embodiments, the high-precision measurement extractor 320 can include a dedicated parameter calculation circuit and statistical analysis hardware that compute pupillary diameter and/or related geometric parameters (e.g., ellipse-fit parameters and/or pupil area) using specialized mathematical processing engines and coordinate transformation circuits. In some cases, the high-precision measurement extractor 320 may implement a dedicated uncertainty quantification circuit and measurement validation hardware that can achieve precise parameter extraction through specialized error analysis processors and accuracy assessment engines. In embodiments, the high-precision measurement extractor 320 can incorporate a dedicated calibration circuit and scaling computation hardware configured to ensure measurement consistency through specialized normalization processors and reference comparison circuits. In some embodiments, the high-precision measurement extractor 320 may transmit measured parameter data, e.g., through dedicated output buffer circuits to the confidence score generator 322. In some embodiments, the high-precision measurement extractor 320 may provide measurement uncertainty data to the quality enhancement validator 324, e.g., through a dedicated reliability interface circuit that can be independent of a data pathway established between the high-precision measurement extractor 320 and the confidence score generator 322. In embodiments, the extracted metrics include high-precision pupil diameter measurements and related geometric parameters with associated uncertainty estimates.

In embodiments, the confidence score generator 322 may comprise a statistical processing circuit and reliability assessment hardware configured to receives measurement data from the high-precision measurement extractor 320, quality metrics from the enhanced image reconstructor 316, and boundary quality data from the pupil boundary detector 318 through dedicated multi-input interface circuits. In embodiments, the confidence score generator 322 can include dedicated scoring algorithm circuits and confidence computation hardware that produce reliability metrics using specialized statistical processing engines and quality assessment circuits. The confidence score generator 322 utilizes dedicated weighting circuits and composite scoring hardware combined with uncertainty propagation processing circuits to generate comprehensive confidence measures through real-time quality analysis engines. In embodiments, the confidence score generator 322 can incorporate dedicated validation circuits and reliability threshold hardware that ensure confidence accuracy through specialized verification processors and consistency monitoring circuits. The confidence score generator 322 generates confidence metric data through dedicated output formatting circuits that feed the quality enhancement validator 324 while providing confidence information through dedicated result interface pathways for final system output. In some embodiments, the quality enhancement validator 324 may be configured to validate results received from the confidence score generator 322, e.g., through real-time verification engines. As used herein, a confidence score comprises a quantitative measure of reliability associated with extracted pupillary measurements and/or derived scores, determined based on measurement uncertainty, boundary quality, noise levels, and consistency across multiple frames.

In embodiments, the quality enhancement validator 324 may comprise a validation processing circuit and quality verification hardware configured to performs final verification using enhanced results from the enhanced image reconstructor 316, measurement reliability data from the high-precision measurement extractor 320, noise metrics from the temporal averaging engine 308 and noise reduction filter 314, and confidence scores from the confidence score generator 322 through dedicated multi-input validation interface circuits. In embodiments, the quality enhancement validator 324 can include dedicated consistency checking circuits and validation algorithm hardware that ensure measurement validity using specialized verification engines and quality control processors. The quality enhancement validator 324 implements dedicated threshold comparison circuits and acceptance criteria hardware combined with comprehensive quality assessment processing circuits to validate final results through real-time verification engines. In embodiments, the quality enhancement validator 324 can incorporate dedicated reporting circuits and result certification hardware that provide final validation confirmation through specialized quality assurance processors and compliance monitoring circuits. The quality enhancement validator 324 generates validated output data through dedicated result interface circuits that provide certified enhanced pupillometry measurements with associated quality metrics and validation status indicators.

In some embodiments, the processing unit 106 may execute machine readable instructions to implement the quality control system show in FIG. 4. In some embodiments, the quality control system shown in FIG. 4 may comprise a comprehensive quality control framework for one or both pre-recording and post-recording quality assurance in pupillometry measurements performed by the computing system 100.

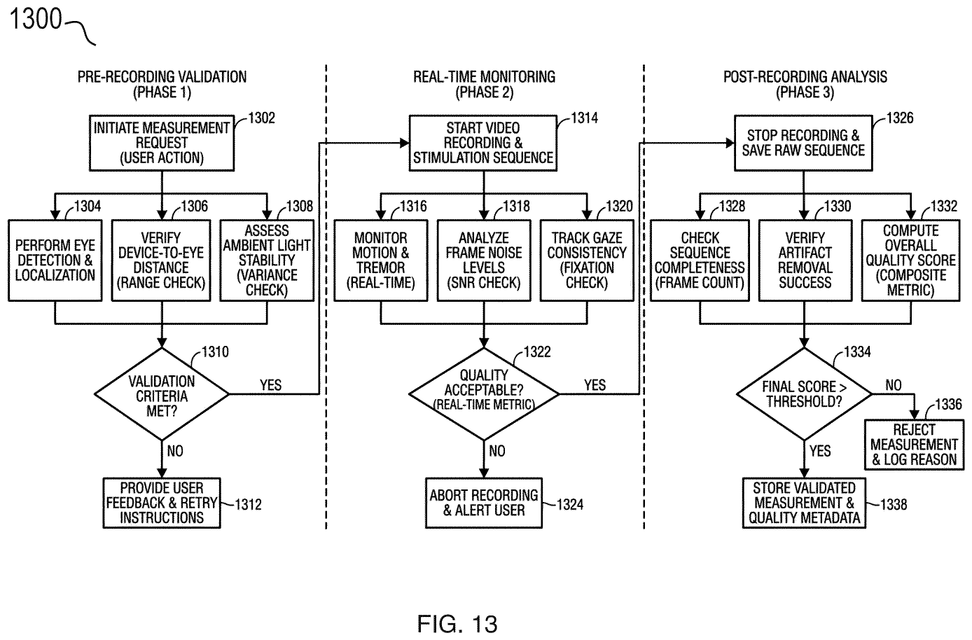

In embodiments, the eye detection processing circuit 400 may initiate the quality control process by analyzing incoming video frames to identify and locate ocular structures within the field of view. In embodiments, the eye detection processing circuit 400 can be associated with an exemplary method flow described with respect to FIG. 13. In embodiments, the eye detection processing circuit 400 can implement convolutional neural network architectures specifically trained to distinguish eye features from surrounding facial anatomy, background elements, and potential obstructions. The eye detection processing circuit 400 employs real-time image processing techniques that analyze pixel intensity patterns, edge gradients, and morphological characteristics to establish precise boundaries of the eye region. In embodiments, the eye detection processing circuit 400 can generate confidence scores ranging from 0.0 to 1.0, where values above 0.85 typically indicate successful eye detection suitable for subsequent processing stages.

In embodiments, as indicated by the arrows in FIG. 4, incoming video frames may be analyzed by the eye detection processing circuit 400, to detect an eye region, and one or more quality input components configured to measure and/or compute complementary parameters such as, distance (e.g., using a distance measurement processing circuit 402), position (e.g., using a positioning verification processor 404), illumination stability (e.g., using an ambient stability monitoring circuit 406), and motion and/or occlusion (e.g., using a motion artifact detection circuit 408). In embodiments, outputs of the quality input components can be integrated by a pre-recording validation gate 410 to determine whether to allow capture, to provide guidance via a real-time feedback generation circuit 412, and/or to inhibit capture. In embodiments, a recording initiation control circuit 414 may begin recording when some of the measured conditions satisfy a specified criterion, and a post-recording analysis and quality scoring pipeline (e.g., a quality score calculation circuit 424, a confidence metric generation circuit 426, and a data acceptance/rejection logic circuit 428) determines whether results can be accepted for display, storage, and/or export.

In embodiments, the distance measurement processing circuit 402 operates in conjunction with the eye detection processing circuit 400 to determine the spatial relationship between the imaging device 102 and the patient's eye 122. In embodiments, the distance measurement processing circuit 402 can utilize multiple complementary techniques, including iris diameter analysis, autofocus lens position calibration, facial landmark detection, and hardware-based sensing methods such as LiDAR or TrueDepth capabilities when available on the device. The distance measurement processing circuit 402 implements mathematical models that convert pixel measurements to physical distances by leveraging the biological constraint that human iris diameter averages approximately 11.4 millimeters across populations. In embodiments, the distance measurement processing circuit 402 can achieve accuracy within ±2 millimeters across a working range of about 150 to about 350 millimeters. However, these values are provided as examples, and actual performance may vary based on environmental conditions and device specifications.

In embodiments, the positioning verification processor 404 may monitor (e.g., continuously monitor) a geometric relationship between the device (e.g., the imaging device 102 of the computing system 100) and the subject or the patient's eye 122 to ensure optimal measurement conditions. In embodiments, the positioning verification processor 404 can analyze frame-to-frame variations in eye position, orientation angles, and apparent size to detect deviations from ideal positioning parameters. In some cases, the positioning verification processor 404 may use a coordinate transformation matrice and geometrical analysis to quantify angular misalignment and lateral displacement. In embodiments, the positioning verification processor 404 can generate real-time positioning guidance signals when the device orientation exceeds predetermined thresholds, such as angular deviations greater than 15 degrees from perpendicular alignment or lateral displacement exceeding 5 millimeters from the center position. However, these values serve as examples and may be adjusted based on specific implementation requirements.

In embodiments, the ambient light stability monitoring circuit 406 may track environmental illumination conditions throughout the measurement process to identify variations that could affect pupillary response measurements. In embodiments, the ambient light stability monitoring circuit 406 can integrate data from dedicated photodiode sensors, camera exposure parameters, and frame intensity analysis to generate comprehensive lighting assessments. The ambient light stability monitoring circuit 406 implements temporal filtering techniques to distinguish between gradual environmental changes and rapid fluctuations caused by external light sources or shadows. In embodiments, the ambient light stability monitoring circuit 406 can detect illumination changes as small as 10 lux within a measurement timeframe of 5 seconds, enabling the system to flag recordings potentially compromised by unstable lighting conditions. However, these sensitivity specifications are provided as examples.

In embodiments, the motion artifact detection circuit 408 may analyze video sequences to identify and quantify unwanted movement that could compromise measurement accuracy. In embodiments, the motion artifact detection circuit 408 can implement optical flow analysis, feature tracking techniques, and frame difference calculations to characterize both device movement and subject head motion. The motion artifact detection circuit 408 processes accelerometer and gyroscope data from the mobile device to distinguish between intentional positioning adjustments and uncontrolled tremor or shake. In embodiments, the motion artifact detection circuit 408 can establish motion thresholds based on statistical analysis of movement patterns, typically flagging recordings with peak acceleration values exceeding 0.5 g or angular velocity changes greater than 10 degrees per second. However, these thresholds are example and may be modified based on clinical requirements.

In embodiments, a pre-recording validation gate 410 integrates outputs from the eye detection processing circuit 400, distance measurement processing circuit 402, positioning verification processor 404, ambient light stability monitoring circuit 406, and motion artifact detection circuit 408 to make binary decisions about recording initiation. In embodiments, the pre-recording validation gate 410 can implement weighted scoring techniques where each input parameter contributes to an overall readiness score ranging from 0 to 100. The pre-recording validation gate 410 employs decision logic that requires simultaneous satisfaction of multiple criteria before enabling data acquisition. In embodiments, the pre-recording validation gate 410 can require eye detection confidence above 0.90, distance measurements within the 150-350 millimeter range, positioning angles within ±10 degrees, ambient light stability within ±20 lux variation, and motion levels below defined thresholds. However, these criteria represent examples that may be adjusted for specific use cases.

In embodiments, a real-time feedback generation circuit 412 processes validation gate outputs to provide immediate guidance to device operators. In embodiments, the real-time feedback generation circuit 412 can generate visual indicators, audio prompts, and haptic signals to guide users toward optimal measurement conditions. The real-time feedback generation circuit 412 implements state machine logic that translates technical parameter deviations into actionable user instructions. In embodiments, the real-time feedback generation circuit 412 can display color-coded indicators where green represents acceptable conditions, yellow indicates marginal performance requiring attention, and red signals conditions incompatible with accurate measurement, providing intuitive guidance that reduces operator training requirements.

In embodiments, a recording initiation control circuit 414 manages the transition from preparation to active data acquisition based on validation gate approval. In embodiments, the recording initiation control circuit 414 can coordinate camera activation, flash timing, exposure settings, and frame capture sequencing to ensure synchronized data collection. The recording initiation control circuit 414 implements timing protocols that account for device latencies and settling times. In embodiments, the recording initiation control circuit 414 can introduce programmable delays ranging from about 10 to about 1000 milliseconds between validation approval and recording start to allow for system stabilization. However, these timing parameters are provided as examples.

In embodiments, a post-recording analysis circuit 416 examines completed measurement sequences to verify data quality and extract relevant parameters. In embodiments, the post-recording analysis circuit 416 can implement comprehensive quality assessment techniques, including temporal consistency analysis, signal-to-noise ratio evaluation, and physiological plausibility checks. The post-recording analysis circuit 416 processes the entire recorded sequence to identify segments compromised by artifacts or technical issues. In embodiments, the post-recording analysis circuit 416 can generate detailed quality reports that quantify measurement uncertainty and provide confidence intervals for extracted parameters, enabling clinical users to make informed decisions about result reliability.

In embodiments, a blink detection processing circuit 418 identifies eyelid closure events that temporarily occlude the pupil during measurement sequences. In embodiments, the blink detection processing circuit 418 can analyze frame-to-frame changes in visible pupil area, iris contrast, and eyelid position to distinguish between voluntary blinks and measurement artifacts. The blink detection processing circuit 418 employs temporal pattern recognition to characterize normal blink dynamics versus pathological conditions. In embodiments, the blink detection processing circuit 418 can detect blink events with onset precision within ±16 milliseconds when operating at 60 frames per second, enabling accurate exclusion of compromised data segments, though this timing specification is exemplary.

In embodiments, a correlation validation circuit 420 examines relationships between stimulation intensity and pupillary response to verify physiological consistency. In embodiments, the correlation validation circuit 420 can calculate cross-correlation coefficients between flash illumination profiles and measured pupil diameter changes to identify recordings where expected stimulus-response coupling is absent or abnormal. The correlation validation circuit 420 implements statistical analysis techniques to establish confidence bounds for normal pupillary responses. In embodiments, the correlation validation circuit 420 can flag measurements where stimulus-response correlation falls below 0.7, indicating potential technical or physiological issues that warrant clinical attention. However, this threshold value is provided as an example.

In embodiments, an artifact flagging processing circuit 422 systematically identifies and categorizes various forms of measurement contamination. In embodiments, the artifact flagging processing circuit 422 can detect corneal reflections, eyelash occlusions, mascara interference, and electronic noise patterns through pattern recognition techniques. The artifact flagging processing circuit 422 employs machine learning models trained on diverse artifact examples to achieve robust detection across different subject populations and environmental conditions. In embodiments, the artifact flagging processing circuit 422 can classify artifacts into categories such as temporary occlusions requiring frame exclusion versus systematic issues requiring measurement repetition, providing specific guidance for result interpretation.

In embodiments, a quality score calculation circuit 424 may synthesize outputs from multiple validation and analysis circuits to generate one or more quality metrics (e.g. a quality score). In embodiments, the quality score calculation circuit 424 can implement weighted averaging techniques where different quality factors contribute proportionally to overall assessment scores. In some cases, the quality score calculation circuit 424 may process inputs including detection confidence, positioning stability, lighting consistency, motion levels, blink frequency, correlation strength, and artifact severity to generate standardized quality scores. In embodiments, the quality score calculation circuit 424 can produce scores ranging from 0 to 100, where values above 80 typically indicate high-quality measurements suitable for clinical interpretation, though these scoring ranges are exemplary.

In embodiments, a confidence metric generation circuit 426 translates quality scores into statistical confidence intervals and uncertainty estimates. In embodiments, the confidence metric generation circuit 426 can implement Bayesian inference techniques to propagate measurement uncertainties through parameter extraction processes. The confidence metric generation circuit 426 generates probabilistic assessments of measurement reliability based on observed or determined quality factors or scores. In embodiments, the confidence metric generation circuit 426 can provide confidence intervals with coverage probabilities of 95% for extracted pupillary parameters, enabling clinical users to assess measurement precision. However, this confidence level is provided as an example.

In embodiments, a data acceptance/rejection logic circuit 428 makes final determinations about measurement validity based on integrated quality assessments. In embodiments, the data acceptance/rejection logic circuit 428 can implement multi-criteria decision techniques that account for clinical safety requirements and measurement precision goals. The data acceptance/rejection logic circuit 428 employs configurable thresholds that can be adjusted based on clinical applications and institutional requirements. In embodiments, the data acceptance/rejection logic circuit 428 can automatically flag measurements falling below acceptance criteria while providing detailed justification for rejection decisions, supporting quality assurance protocols and regulatory compliance requirements. The decision logic accounts for the criticality of different measurement parameters and clinical contexts to ensure appropriate sensitivity and specificity in quality determinations.

FIG. 6 is a block diagram of a multi-parameter confounder analysis and correction system that may be implemented by the processing unit 106 to identify and, quantify confounding effects and correct or adjust a pupillary parameter measured and/or generated, e.g., by the light invariant pupil reactivity score calculation system shown in FIG. 2. In some embodiments, the multi-parameter confounder analysis and correction system shown in FIG. 6 may comprise a confounder identification engine 616 configured to receive confounder-related data from one or more confounder assessment systems (e.g., upstream confounder assessment systems) and identify one or more confounding factors that may influence a pupillary measurement, e.g., a pupillary measurement performed by the computing system 100. In some embodiments, the confounder identification engine 616 may receive confounder-related data from one or more of a demographic data collector 600, medication history examination interface 606, gaze direction tracker 610, accommodation state monitor 612, environmental evaluation interface 614, and other systems that may store and provide confounder-related data. In some embodiments, confounder-related data may comprise data that nay be used to identify and adjust for confounding effects during a statistical analysis, ensuring that observed associations more accurately reflect true causal relationships.

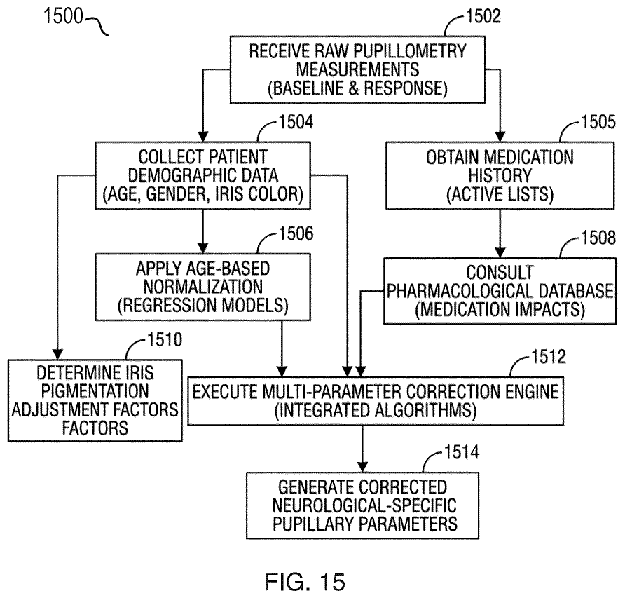

In some embodiments, the demographic data collector 600 that operates as a specialized data acquisition interface and can be configured to receive and process patient demographic information relevant to pupillary response normalization. In embodiments, the demographic data collector 600 interfaces directly with the user through a graphical user interface displayed on the mobile computing device, presenting input fields for manual entry of demographic parameters including age, gender, iris color, and other physiologically relevant characteristics. In embodiments, the demographic data collector 600 can execute retrieval protocols through standardized application programming interfaces that establish secure connections with electronic medical record systems, automatically extracting patient demographic information to minimize manual data entry. In embodiments, the demographic data collector 600 transmits structured demographic data packets to an age normalization processor 602 through dedicated data buses within the processing architecture. In embodiments, an exemplary method flow for confounder correction is described with respect to FIG. 15.

In embodiments, as indicated by the arrows in FIG. 6, patient context captured by the demographic data collector 600 is provided through one or more normalization and correction processors (e.g., an age normalization processor 602 and a gender adjustment processor 604), and additional confounder context (e.g., medication history examination interface 606, drug interaction detector 608, and video-derived context such as gaze direction tracker 610 and accommodation state monitor 612) is combined to produce confounder-aware corrected pupillometry outputs and/or decision support.

In embodiments, an age normalization processor 602 receives the demographic data and executes computational operations specifically designed to compensate for age-related physiological variations in pupillary response parameters. In embodiments, the age normalization processor 602 implements processing circuits that apply mathematical transformation functions to raw pupillary measurements, adjusting these measurements based on established age-dependent relationships between pupil size, constriction amplitude, and response velocities. In embodiments, the age normalization processor 602 accesses normative database tables stored in memory that contain stratified reference distributions organized by age ranges and applies scaling transformations to measured pupillary parameters, subtracting age-specific baseline values and dividing by age-specific normalization factors to produce age-invariant measurements. In embodiments, the age normalization processor 602 forwards age-corrected measurement data to a gender adjustment processor 604 through dedicated signal pathways.

In embodiments, a gender adjustment processor 604 receives the age-normalized measurements and executes computational operations that account for gender-based physiological differences in pupillary function. In embodiments, the gender adjustment processor 604 implements processing circuits that access gender-stratified reference data stored in memory and execute comparative analysis operations that evaluate measured pupillary parameters against gender-specific normal ranges. In embodiments, the processing circuits within the gender adjustment processor 604 generate gender-normalized output values by applying multiplicative scaling factors and additive offset corrections derived from population studies. In embodiments, the gender adjustment processor 604 transmits demographically corrected measurement data to a medication history examination interface 606 through internal data pathways.

In embodiments, the medication history examination interface 606 may operate as a specialized processing subsystem configured to receive, parse, and analyze pharmaceutical agent information relevant to pupillary function. In embodiments, the medication history examination interface 606 implements data acquisition circuits that retrieve medication lists from electronic medical record systems through secure API connections and can present interactive displays to healthcare providers, enabling manual entry when automated retrieval is unavailable. In embodiments, the processing architecture within the medication history examination interface 606 maintains a comprehensive pharmaceutical database stored in memory that catalogs known effects of various drug classes on pupillary function, including anticholinergics, opioids, sympathomimetics, and sedatives. In embodiments, the medication history examination interface 606 performs classification operations that categorize each identified medication according to its potential influence on pupillary measurements and forwards structured medication influence data to a drug interaction detector 608.

In embodiments, a drug interaction detector 608 receives the medication influence data and implements specialized processing circuits that identify potential synergistic or antagonistic interactions between multiple pharmaceutical agents affecting pupillary function. In embodiments, the drug interaction detector 608 executes combinatorial analysis operations that evaluate all possible combinations of identified medications, accessing a drug interaction database stored in memory. In embodiments, the drug interaction detector 608 implements scoring mechanisms that quantify the expected compound effect of multiple medications on pupillary measurements and generate flags or alerts when detected interactions exceed predetermined significance thresholds. In embodiments, the drug interaction detector 608 transmits medication interaction analysis results to a gaze direction tracker 610 through internal data buses.

In embodiments, the gaze direction tracker 610 operates as a computer vision processing subsystem that executes real-time analysis of eye orientation and fixation characteristics during pupillometry measurements. In embodiments, the gaze direction tracker 610 receives video frame data from the camera system and implements pupil center detection circuits that identify the spatial position of the pupil centroid within the imaging field. In embodiments, the gaze direction tracker 610 implements geometric transformation circuits that convert two-dimensional pupil positions into three-dimensional gaze vector estimates, taking into account the camera perspective, eye anatomy, and head position. In embodiments, the processing architecture executes deviation calculations that quantify the angular difference between the current gaze direction and an ideal straight-ahead fixation target and implements quality scoring operations that evaluate gaze stability. In embodiments, the gaze direction tracker 610 forwards gaze characterization data to an accommodation state monitor 612.

In embodiments, the accommodation state monitor 612 may receive information and executes specialized processing operations that estimate the eye's accommodative focus state during pupillometry measurements. In embodiments, the accommodation state monitor 612 implements lens analysis circuits that examine video frames to detect changes in pupil size or iris configuration associated with accommodation responses. In embodiments, the accommodation state monitor 612 performs distance estimation calculations to determine the subject's viewing distance to the mobile device, accessing distance measurement data from multimodal distance estimation systems that include autofocus sensors, iris diameter analysis, LiDAR, or TrueDepth capabilities. In embodiments, the processing circuits implement accommodation influence quantification techniques that estimate the magnitude of pupil size change attributable to accommodative activity and generate correction factors applied to measured pupillary parameters. In embodiments, the accommodation state monitor 612 transmits accommodation characterization data to an environmental factor evaluation interface 614.

In embodiments, the environmental factor evaluation interface 614, which may operates as a comprehensive processing subsystem, may be configured to receive, integrate, and analyze multiple environmental parameters affecting pupillary measurements. In some cases, the environmental parameters may comprise ambient light, temperature, acceleration, motion, and the like. In embodiments, the processing architecture. In embodiments, the environmental factor evaluation interface 614 implements sensor data acquisition circuits that receive inputs from various environmental monitoring systems, including ambient light sensors, temperature sensors, and accelerometers for motion detection. In embodiments, the processing architecture performs temporal stability analysis on environmental parameters, calculating variance or drift metrics that indicate whether measurement conditions remained sufficiently stable during the pupillometry recording. In embodiments, the environmental factor evaluation interface 614 executes correlation analysis operations that examine relationships between environmental variations and observed pupillary response characteristics. In embodiments, the environmental factor evaluation interface 614 forwards comprehensive environmental characterization data to a confounder identification engine 616.

In embodiments, a confounder identification engine 616 may receives data from one or more upstream confounder assessment systems, described above or other system, and may implement integrated processing circuits configured to synthesize multidimensional information (e.g., confounder-related data) to identify which one or more confounding factors that can influence a pupillary measurements (e.g., a currently pupillary measurement), by a amount or level exceeding a threshold level. In some embodiments, the confounder identification engine 616 may identify a confounding factor as significant when an estimated impact of the confounder on a pupillary measurement (e.g., one or more pupillary response parameters and/or a normalized score output 230) exceeds a predefined significance threshold and/or when statistical significance testing indicates a meaningful effect. In embodiments, the predefined significance threshold is configurable and can be selected based on one or more of an uncertainty metric propagated through the correction pipeline and a clinical relevance criterion. In embodiments, the confounder identification engine 616 executes multivariate analysis operations that evaluate the relative contribution of demographic factors, pharmaceutical influences, gaze characteristics, accommodation state, and environmental conditions to observed pupillary response patterns. In embodiments, the confounder identification engine 616 performs significance testing operations that quantify the statistical likelihood that each identified confounder has a meaningful effect on the measurements. In embodiments, the processing circuits generate structured confounder characterization outputs that specify which correction approaches should be applied and in what sequence, forwarding these directives to a correction technique selection interface 618.

In embodiments, a correction technique selection interface 618 receives the confounder characterization data and implements decision logic circuits that determine optimal correction strategies for each identified confounding factor. In embodiments, the correction technique selection interface 618 accesses a library of correction techniques stored in memory, with each technique designed to address specific confounder types through different mathematical transformation approaches, including linear adjustments, nonlinear scaling, statistical normalization, or machine learning-based correction models. In embodiments, the correction technique selection interface 618 performs parameter optimization calculations that tune correction technique parameters based on the specific characteristics of detected confounders. In embodiments, the correction technique selection interface 618 transmits correction technique specifications to a normalization processor 620.

In embodiments, a normalization processor 620 receives correction specifications and raw pupillometry measurements, then executes the selected correction transformations through dedicated arithmetic processing circuits. In embodiments, the normalization processor 620 implements sequential correction operations that systematically remove confounding influences from measured pupillary parameters. These operations apply demographic corrections first, followed by pharmacological adjustments, gaze corrections, accommodation corrections, and environmental compensations in an optimized processing sequence. In embodiments, the normalization processor 620 executes uncertainty propagation calculations that track how measurement uncertainties and correction uncertainties combine throughout the normalization process. In embodiments, the processing circuits generate intermediate corrected measurements after each correction stage and final, fully corrected measurements after all selected corrections have been applied, forwarding these to a clinical relevance filter 622.

In embodiments, a clinical relevance filter 622 receives the corrected measurements and implements processing circuits that evaluate whether the confounder-corrected pupillary changes reflect clinically significant neurological alterations. In embodiments, the clinical relevance filter 622 executes threshold comparison operations that evaluate corrected measurements against clinically established boundaries between normal and abnormal pupillary function. In embodiments, the clinical relevance filter 622 implements temporal analysis operations when longitudinal measurement series are available, evaluating whether changes over time in corrected measurements exceed normal day-to-day variation. In embodiments, the clinical relevance filter 622 generates binary classification outputs indicating clinical significance and forwards both the corrected measurements and clinical significance indicators to a corrected measurement output system 624.

In embodiments, a corrected measurement output system 624 may receive the confounder-corrected pupillary parameters, e.g., from the clinical relevance filter 622, and implement formatting and presentation processing circuits to structure the received information (e.g., confounder-corrected pupillary parameters) for clinical use. In embodiments, the corrected measurement output system 624 executes data packaging operations that combine corrected Pupil Reactivity scores, corrected diameter measurements, corrected velocity parameters, and other pupillary metrics into standardized output formats suitable for display on the mobile device, transmission to electronic medical records, or storage in research databases. In embodiments, the corrected measurement output system 624 can generate graphical visualizations that present corrected measurements alongside indicators showing which confounders were detected and corrected. In embodiments, the corrected measurement output system 624 transmits formatted corrected measurements to a decision support generator 626 and external systems.

In embodiments, a decision support generator 626 receives the corrected measurements and implements clinical decision logic circuits that provide contextualized interpretation guidance to healthcare providers. In embodiments, the decision support generator 626 executes comparative analysis operations that evaluate corrected measurements against normative reference databases stratified by age, gender, diagnosis, and other relevant factors, generating percentile rankings or severity scores. In embodiments, the decision support generator 626 performs alert generation operations that produce notifications when corrected measurements cross critical thresholds, such as Pupil Reactivity scores falling below 3.0, which indicates severe neurological impairment, enabling timely clinical intervention. This numerical value serves merely as an illustrative example. In embodiments, the processing circuits generate structured decision support outputs including recommended actions, suggested follow-up timing, and relevant clinical considerations based on the confounder-corrected pupillometry results.

In embodiments, the comprehensive confounder correction framework illustrated in FIG. 6 enables the computational pupillometry system to isolate neurologically-relevant pupillary changes from the numerous physiological, pharmacological, and environmental factors that influence pupillary measurements in real-world clinical settings.

Regarding FIG. 7, a saturation function mathematical framework 700 implements the core computational approach for generating light-invariant PuRe scores from pupillary measurements. In embodiments, the saturation function mathematical framework 700 combines ambient illumination estimates, calibration data, and measured pupil constriction metrics to produce normalized scores that exhibit minimal correlation with environmental lighting conditions while preserving neurological sensitivity.

In embodiments, as indicated by the arrows in FIG. 7, the framework receives an estimated ambient illumination input 701 (L) from ambient light estimation subsystems and a pupil constriction measure 703 (A) from pupillary parameter measurement engines. In embodiments, calibration data 705 encoding relationships between illumination levels and model parameters are provided to a parameter determination module 710 that computes illumination-conditioned parameters. In embodiments, the parameter determination module 710 outputs a rate parameter k via a rate parameter k calculation 712 and a shape parameter b via a shape parameter b calculation 714. In embodiments, these parameters are provided to a saturation function computation kernel 720 that evaluates the saturation function, and the result is processed by a score scaling and normalization block 730 to generate a final normalized PuRe score 740 on a 0 to PuReMAX scale.

In embodiments, an estimated ambient illumination input 701 (L) represents the environmental lighting conditions quantified during pupillary assessment. In embodiments, the estimated ambient illumination 701 can be expressed in lux units or as a logarithmic transform log₁₀(lux) to linearize relationships across the photopic range from about 0.1 lux to about 3000 lux (and in some embodiments up to about 10,000 lux). In embodiments, the estimated ambient illumination 701 is derived from video frame analysis, dedicated light sensor measurements, camera settings (exposure, gain, ISO), and/or machine learning models that predict ambient light from statistical features extracted from pre-flash and flash-transition frames.

In embodiments, a pupil constriction measure 703 (A) quantifies the magnitude of the pupillary light reflex response. In embodiments, the pupil constriction measure 703 can be expressed as a relative constriction percentage (CAMP/INIT×100%) or as an absolute constriction amplitude (CAMP=INIT−MIN), where INIT denotes the initial baseline pupil diameter prior to stimulus and MIN denotes the minimum pupil diameter after stimulus. In embodiments, the pupil constriction measure 703 is extracted from pupillary parameter measurement engines that analyze video sequences captured during controlled flash stimulation, performing pupil segmentation, diameter measurement, and temporal filtering to reduce frame-to-frame noise.

In embodiments, calibration data 705 encodes empirically determined relationships between ambient illumination levels and saturation function parameters. In various implementations, It implies that the relationships (which were empirically determined calibration data may comprise a mathematical relation, an algorithmic, or a data structure capturing the a relation relationships between ambient illumination levels and saturation function parameters. For example, calibration data 705 may comprise a lookup table, a set of coefficients in an equation, a machine learning model, a mapping function or the like.

In embodiments, calibration data 705 encodes empirically determined relationships between ambient illumination levels and saturation function parameters.

In embodiments, the calibration data 705 is derived from normative datasets collected from healthy control subjects without neurological impairment who are not under sedatives or anesthetics, measured across multiple ambient illumination conditions. In embodiments, the calibration data 705 can include regression coefficients, lookup tables, or trained model parameters that describe how mean constriction amplitudes and saturation function parameters vary with log₁₀(lux). In embodiments, the calibration data 705 resides in non-volatile memory storage with data structures organized for rapid retrieval based on lighting level indices. In embodiments, calibration data 705 encodes empirically determined relationships between ambient illumination levels and saturation function parameters by storing such relationships in one or more machine-readable forms, including lookup tables, mathematical coefficients, mapping functions, or trained model parameters.

In embodiments, a parameter determination module 710 computes the illumination-conditioned saturation function parameters k and b that define the mapping from constriction amplitude to normalized score at the current ambient lighting level. In embodiments, the parameter determination module 710 retrieves calibration data 705 and applies mathematical transformations based on the estimated ambient illumination 701 to determine parameters appropriate for prevailing lighting conditions. The parameter determination module 710 employs processing circuits that execute logarithmic, exponential, and interpolation calculations using CPU floating-point units, GPU compute capabilities, or specialized mathematical coprocessors with 16-128 bit precision.

In embodiments, a rate parameter k calculation 712 determines the rate parameter k that controls the steepness and scaling of the saturation function. In embodiments, the rate parameter k is computed as a function of ambient illumination: k=g(L), where g(·) represents an illumination-dependent mapping derived from calibration data 705. In embodiments, k can be determined using linear interpolation between reference k values at dim and bright illumination levels, polynomial approximations, spline interpolation (degrees 1-10), or lookup table methods (100-100,000 entries). Typically, k values range from about 0.01 to about 1.0, although these ranges serve as examples.

In embodiments, a shape parameter b calculation 714 determines the shape parameter b that controls the nonlinearity and curvature of the saturation function. In embodiments, the shape parameter b is computed as a function of ambient illumination: b=h(L), where h(·) represents an illumination-dependent mapping. In embodiments, b can be computed using the formula b=[In(−In(d₁))−In(−In(d₂))]/[In(Δmean)−In(Δthreshold)], where d₁ and d₂ represent fractional PuRe values (example values d₁≈0.2 and d₂≈0.8), and Δmean and Δthreshold are illumination-dependent constriction amplitudes retrieved from calibration data 705. Typically, b values range from about 0.5 to about 2.0, although these ranges serve as examples.

In embodiments, a saturation function computation kernel 720 implements the core mathematical transformation that maps pupil constriction measures to saturation values exhibiting light-invariant characteristics. In embodiments, the saturation function computation kernel 720 evaluates the expression f (Δ; k, b)=1−exp(−k·Δ^{b}), where Δ represents the pupil constriction measure 703, k is the rate parameter from the rate parameter k calculation 712, b is the shape parameter from the shape parameter b calculation 714, and exp denotes the natural exponential operator. The saturation function computation kernel 720 comprises dedicated arithmetic circuits that perform power operations (Δ^{b}) and exponential evaluations through optimized mathematical techniques including Taylor series approximations, CORDIC algorithms, or hardware transcendental function units. In embodiments, the saturation function computation kernel 720 can implement alternative saturation functions including sigmoid (logistic) forms, hyperbolic tangent functions, piecewise linear approximations, or lookup-table-based evaluations.

In embodiments, a score scaling and normalization block 730 transforms the saturation value from the saturation function computation kernel 720 into a final normalized PuRe score on a standardized scale. In embodiments, the score scaling and normalization block 730 implements the scaling equation 732 PuRe=PuReMAX×f (Δ; k, b), where PuReMAX is a maximum score parameter 734 typically set to 5.0 to yield scores on a 0-5 scale. In embodiments, the score scaling and normalization block 730 can apply linear scaling, apply minimum-nonzero coercion to prevent underflow, round outputs to discrete intervals (e.g., 0.1 steps), and override the mapping when Δ=0 to output PuRe=0 indicating no detected constriction. In embodiments, the score scaling and normalization block 730 executes on CPU arithmetic logic units or GPU cores with floating-point precision ranging from 16 to 128 bits.

In embodiments, a final normalized PuRe score 740 represents the output of the saturation function mathematical framework 700 on a standardized scale (typically 0 to 5 or 0 to PuReMAX). In embodiments, the final normalized PuRe score 740 exhibits light-invariant properties, demonstrating minimal correlation (|r|<0.15, and preferably |r|<0.1) with ambient light levels across the photopic range while preserving sensitivity to neurologically relevant changes. In embodiments, the final normalized PuRe score 740 is output to user interfaces for clinical interpretation, stored in electronic medical records, compared against clinical thresholds (e.g., PuRe≤3.0) for classification, and/or used in multi-parameter diagnostic models for intracranial pressure assessment, traumatic brain injury evaluation, or neurological status monitoring.

In embodiments, a clinical interpretation engine transforms numerical PuRe scores into clinically meaningful assessments, providing contextualized information to support diagnostic decision-making. In embodiments, the clinical interpretation engine can generate textual descriptions categorizing pupillary responses as “brisk,” “normal,” “sluggish,” “minimal,” or “non-reactive” based on score ranges. The clinical interpretation engine comprises processing circuits that execute rule-based inference systems, mapping numerical scores to standardized clinical terminologies. In embodiments, the clinical interpretation engine can incorporate patient-specific contextual factors, including demographic information and medication history, when formulating interpretative assessments. In some cases, the clinical interpretation engine may comprise a clinical threshold comparator 224).

In embodiments, an uncertainty quantifier 722 computes statistical measures characterizing the reliability and precision of calculated PuRe scores. In embodiments, the uncertainty quantifier 722 can determine score standard deviations based on propagated uncertainties from underlying diameter measurements and temporal variability across frame sequences. The uncertainty quantifier 722 employs processing circuits that implement error propagation techniques to track how measurement uncertainties propagate through the saturation function transformation operations.

In embodiments, a confidence interval generator 724 constructs statistical intervals around computed PuRe scores, which quantify the range of plausible true values consistent with the observed measurements. In embodiments, the confidence interval generator 724 can determine 95 percent confidence intervals spanning from PuRe−1.96·SE to PuRe+1.96·SE, where SE represents the standard error provided by the uncertainty quantifier 722. The confidence interval generator 724 comprises processing circuits that execute statistical calculations to determine appropriate interval widths based on measurement sample sizes and desired confidence levels.

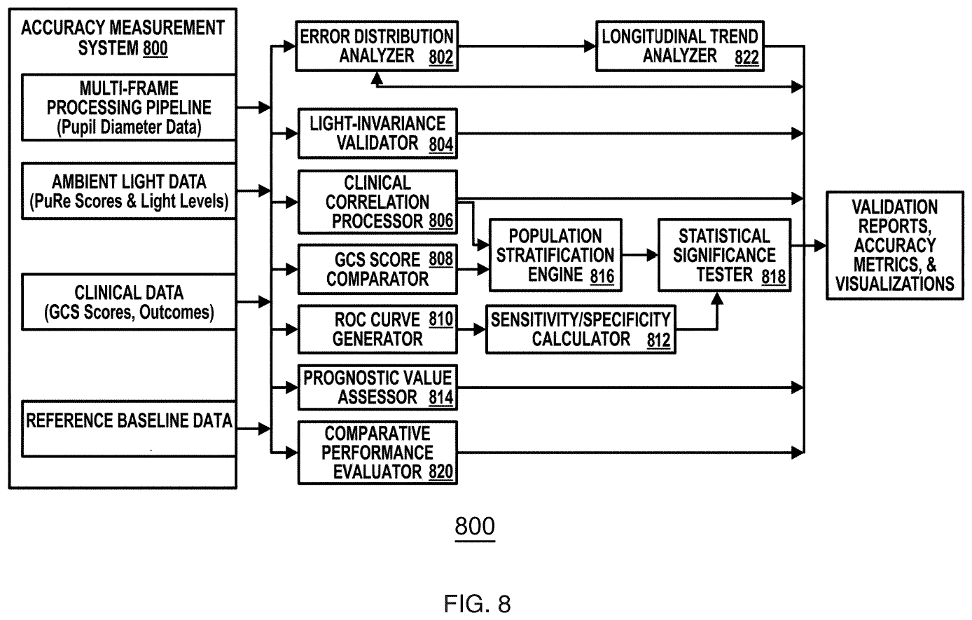

Regarding FIG. 8, an accuracy measurement system 800 provides comprehensive validation capabilities for computational pupillometry measurements. In embodiments, the accuracy measurement system 800 receives pupillary response data and processes this temporal information to demonstrate measurement consistency, clinical correlation, and prognostic value. In some implementations, the clinical validation system 800 may receive inputs including: (i) recording accuracy and quality metrics; (ii) ambient illumination context; (iii) calculated PLR scores for each eye; (iv) contralateral-eye information; (v) clinical metrics such as GCS and ICP obtained from EMR systems; and (vi) baseline normative data. The system outputs validation and context-aware assessments that supplement the primary score.

The accuracy measurement system 800 includes processing circuits that validate technical performance and clinical utility. In embodiments, the accuracy measurement system 800 can integrate multiple processing stages that analyze measurement precision, environmental robustness, clinical relevance, and longitudinal tracking. The accuracy measurement system 800 executes on distributed processing circuits, including validation processors, statistical analysis processors, and database management processors.

In embodiments, as indicated by the arrows in FIG. 8, pupillometry outputs are supplied to the accuracy measurement system 800, which routes such outputs through one or more validation stages (e.g., an error distribution analyzer 802 and a light-invariance validator 804) and generates one or more validation reports, thresholds, and/or performance summaries used to demonstrate technical and clinical utility.

In embodiments, an error distribution analyzer 802 within the accuracy measurement system 800 calculates frame-to-frame measurement precision. The error distribution analyzer 802 includes processing circuits that receive pupil diameter measurements from sequential video frames and compute statistical distributions of measurement error. In embodiments, the error distribution analyzer 802 can apply spectral low-pass filtering processing circuits to pupillogram data to generate smoothed reference trajectories. The error distribution analyzer 802 implements digital filter processing circuits that execute Butterworth filters, Chebyshev filters, or finite impulse response filters to attenuate high-frequency noise. The error distribution analyzer 802 calculates frame-by-frame deviations between raw measurements and filtered trajectories. In embodiments, the error distribution analyzer 802 can generate statistical distribution data structures organized by stratification factors, including iris color, ambient light measurements, and recording quality. The error distribution analyzer 802 computes root mean square error values, mean absolute error metrics, and maximum error values. In embodiments, the error distribution analyzer 802 can achieve measurement accuracies of approximately ±0.025 millimeters, although this exemplary value may vary depending on implementation details and patient characteristics.

In embodiments, a light-invariance validator 804 within the accuracy measurement system 800 processes pupillary measurements captured under varying ambient illumination conditions. The light-invariance validator 804 includes processing circuits that receive pupil reactivity score values alongside corresponding ambient light measurements expressed in lux units and/or transformed values (e.g., log₁₀(lux)) used for statistical analysis. In embodiments, the light-invariance validator 804 can organize measurement data into lighting categories based on ambient light intensity thresholds. The light-invariance validator 804 implements classification processing circuits that classify measurements as dim when the ambient light level falls below 100 lux and as bright when the ambient light level equals or exceeds 100 lux. The light-invariance validator 804 applies statistical correlation analysis processing circuits that compute Pearson and/or Spearman rank correlation coefficients between paired pupil reactivity scores and ambient illumination metrics. In embodiments, the light-invariance validator 804 computes Spearman rank correlation between PuRe and log₁₀(lux) over measurements spanning a practical operating range (e.g., about 0.1 to about 3000 lux, or about 10 to about 1000 lux). In embodiments, the light-invariance validator 804 can achieve absolute Spearman correlation values below approximately 0.15 (and in some datasets below approximately 0.05), indicating weak association between PuRe and ambient illumination. In embodiments, the light-invariance validator 804 additionally applies binned comparisons across illumination regimes and/or statistical tests (e.g., ANOVA or non-parametric equivalents) that show no significant dependence of PuRe on illumination in normative data. The light-invariance validator 804 performs comparative analysis between light-invariant PuRe and traditional parameters (e.g., INIT, CAMP, and/or Δ), and can demonstrate that such traditional parameters exhibit significantly stronger dependence on ambient illumination (e.g., larger correlation coefficients and/or statistically significant differences between illumination bins).

In embodiments, a clinical correlation processor 806 within the accuracy measurement system 800 integrates pupillometry data with established neurological assessment tools. The clinical correlation processor 806 includes processing circuits that receive pupil reactivity score values alongside corresponding Glasgow Coma Scale assessment values. In embodiments, the clinical correlation processor 806 can implement data synchronization processing circuits that match measurements to GCS assessments based on the proximity of timestamps within predetermined temporal windows. The clinical correlation processor 806 applies non-parametric statistical correlation processing circuits that execute Spearman rank correlation calculations by converting both values to rank-ordered sequences. In embodiments, the clinical correlation processor 806 can achieve correlation coefficients exceeding 0.7, such as approximately 0.746; however, the actual correlation strength may vary. The clinical correlation processor 806 calculates statistical significance metrics that generate p-values, achieving p-values below 0.001 in exemplary implementations.

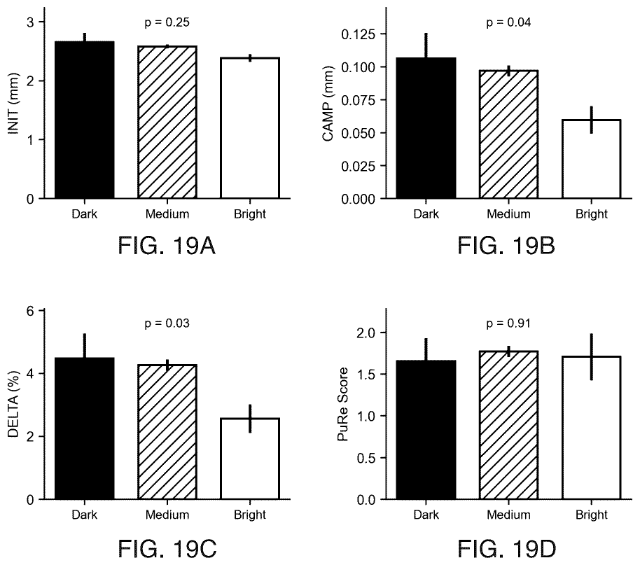

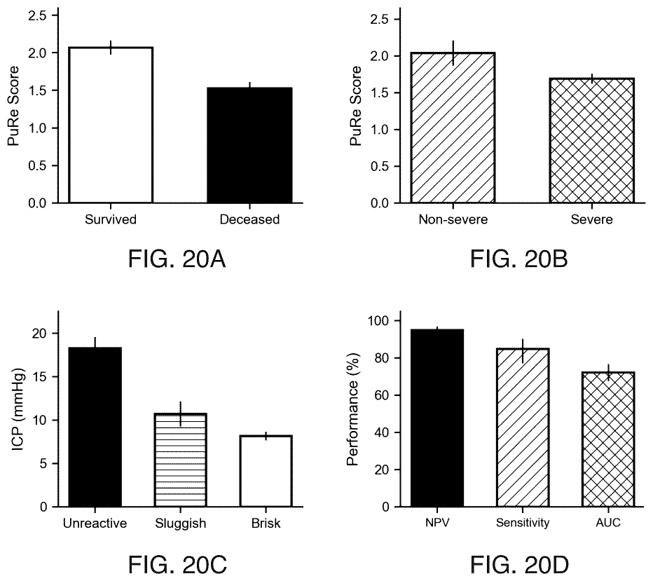

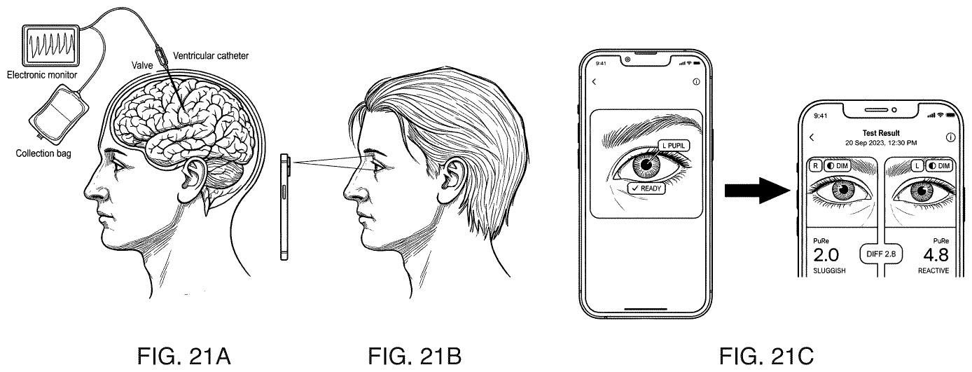

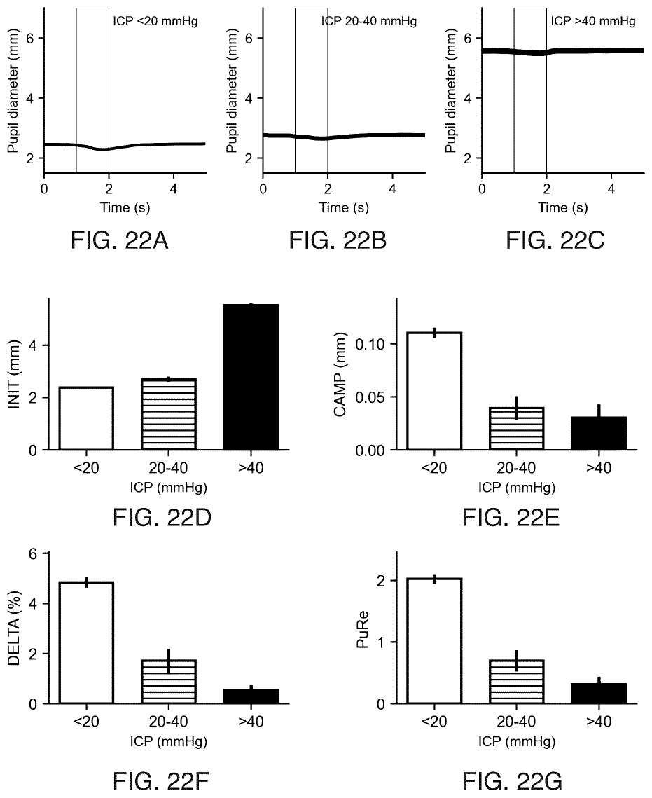

In embodiments, the accuracy measurement system 800 additionally evaluates associations between PuRe scores and intracranial pressure (ICP) measurements and/or intracranial hypertension classifications. In embodiments, intracranial hypertension is defined as an ICP meeting or exceeding a threshold (e.g., ≥20 mmHg). In an exemplary prospective neuro-intensive care study, nineteen patients contributed 731 pupillometry recordings, of which 634 (87%) had concurrent invasive ICP measurements and 112 (18%) showed elevated ICP. In this example, PuRe score exhibited an inverse correlation with ICP (Spearman ρ=−0.17, p<0.001). At an exemplary screening threshold (PuRe≤1.3), sensitivity was 85.7% (95% CI: 78.0-91.0%) and specificity was 61.3% (95% CI: 57.1-65.4%), yielding a negative predictive value (NPV; TN/(TN+FN)) of 95.2% (95% CI: 92.4-97.0%) with an AUC of 0.72 (95% CI: 0.68-0.77). In embodiments, PuRe maintains stable discrimination across ambient illumination conditions (e.g., ANOVA p=0.91) while one or more raw constriction parameters remain illumination-dependent (e.g., CAMP p=0.04, Δp=0.03). In embodiments, such lighting invariance is evaluated using binned illumination categories (e.g., dark, medium, and bright bins defined by one or more lux thresholds), and results are shown as in FIGS. 19A-19D. FIGS. 19A-19B are plots illustrating stability of a normalized pupil reactivity score in a sample neuro-intensive-care-unit patient cohort across ambient illumination conditions compared to one or more raw constriction parameters, in accordance with embodiments of the present disclosure. In embodiments, a screening threshold and/or a model configuration for intracranial hypertension risk classification is selected based on a target NPV for excluding intracranial hypertension (e.g., at least about 90%, such as about 90% to about 99%), and the device outputs the NPV and/or related performance metrics with the intracranial hypertension risk classification. In embodiments, NPV depends on prevalence in an evaluated population and can be computed and/or reported for one or more prevalence regimes. In embodiments, one or more such diagnostic performance metrics (including NPV, sensitivity, and/or AUC) are reported and/or visualized as in FIGS. 20A-20D. FIGS. 20A-20D are plots illustrating diagnostic performance of a normalized pupil reactivity score for intracranial hypertension screening, in accordance with embodiments of the present disclosure. In embodiments, the device presents an explanatory output indicating that a PuRe score exceeding a screening threshold corresponds to low likelihood of intracranial hypertension and can be used as a screening output to rule out intracranial hypertension in a two-tier neuromonitoring workflow. In embodiments, the two-tier workflow comprises using a first tier of high-frequency non-invasive pupillometry screening (e.g., at bedside) and using a second tier of confirmatory evaluation for recordings with depressed and/or deteriorating PuRe (e.g., invasive ICP monitoring, imaging, and/or one or more other non-invasive surrogates), as conceptually illustrated in FIGS. 21A-21C. FIGS. 21A-21C illustrate an example two-tier neuromonitoring method comprising a non-invasive pupillometry device that provides high-frequency screening and an invasive ICP monitor that provides confirmatory measurements in accordance with embodiments of the present disclosure. In embodiments, the device additionally outputs one or more ICP-stratified pupillograms and/or parameter summaries (e.g., PuRe, INIT, CAMP, and/or A) to support clinical interpretation, as shown in FIGS. 22A-22G. FIGS. 22A-22G are plots illustrating pupillograms (FIGS. 22A-22C) and pupillometry parameter differences (FIGS. 22D-22G) across intracranial pressure levels, in accordance with embodiments of the present disclosure. These values are provided as non-limiting examples.

In embodiments, a GCS score comparator 808 within the accuracy measurement system 800 performs temporal alignment between pupil reactivity score trajectories and GCS score progression. The GCS score comparator 808 includes processing circuits that receive timestamped pupil reactivity score measurements and timestamped GCS assessment data. In embodiments, the GCS score comparator 808 can implement temporal alignment processing circuits that organize measurements into chronologically ordered sequences. The GCS score comparator 808 creates data structures storing paired time-series arrays. The GCS score comparator 808 generates synchronized visualization data structures showing parallel tracking through dual-axis line graphs. In embodiments, the GCS score comparator 808 can process example patient data showing progression from approximately 2.5 to approximately 4.3 for pupil reactivity scores and from 3 to 15 for GCS scores, though these represent illustrative examples. The GCS score comparator 808 implements change detection processing circuits that identify critical transition periods.

In embodiments, an ROC curve generator 810 within the accuracy measurement system 800 calculates receiver operating characteristic curves that quantify diagnostic accuracy. The ROC curve generator 810 includes processing circuits that receive measurement datasets containing pupil reactivity score values and corresponding binary classification labels. In embodiments, the ROC curve generator 810 can implement labeling processing circuits that assign severe neurological impairment labels to measurements from patients with GCS at or below 8. The ROC curve generator 810 implements threshold scanning processing circuits that systematically evaluate classification performance across a range of thresholds. The ROC curve generator 810 generates candidate threshold values and iterates through each, classifying measurements accordingly. In embodiments, the ROC curve generator 810 can implement confusion matrix processing circuits that count true positives, false positives, true negatives, and false negatives for each threshold. The ROC curve generator 810 calculates true positive rates and false positive rates. The ROC curve generator 810 calculates area under the curve metrics using numerical integration processing circuits. In embodiments, the ROC curve generator 810 can achieve AUC values exceeding 0.9, such as approximately 0.940, though specific values vary.

In embodiments, a sensitivity/specificity calculator 812 within the accuracy measurement system 800 determines optimal decision thresholds. The sensitivity/specificity calculator 812 includes processing circuits that receive confusion matrix data structures for each threshold. In embodiments, the sensitivity/specificity calculator 812 can calculate sensitivity values by dividing true positive counts by the sum of true positive and false negative counts. The sensitivity/specificity calculator 812 calculates specificity values representing the proportion of actual non-severe cases correctly identified. The sensitivity/specificity calculator 812 implements threshold optimization processing circuits that apply optimization criteria such as maximizing Youden's J statistic. In embodiments, the sensitivity/specificity calculator 812 can identify that a threshold at or below 3.0 achieves approximately 84.8% sensitivity and approximately 90.0% specificity, although these exemplary values may vary. The sensitivity/specificity calculator 812 computes overall accuracy metrics of approximately 86.0%.

In embodiments, a prognostic value assessor 814 within the accuracy measurement system 800 evaluates the capability to predict patient outcomes. The prognostic value assessor 814 includes processing circuits that receive longitudinal pupil reactivity score data alongside ultimate patient outcome data. In embodiments, the prognostic value assessor 814 can implement outcome data retrieval processing circuits that query electronic medical record systems. The prognostic value assessor 814 creates linked data structures that associate early measurements with ultimate outcomes. The prognostic value assessor 814 utilizes survival analysis processing circuits, including Kaplan-Meier estimator processing circuits, to calculate survival probability functions. In embodiments, the prognostic value assessor 814 can implement Cox proportional hazards model processing circuits that estimate hazard ratios. The prognostic value assessor 814 calculates median pupil reactivity scores for different outcome groups, such as approximately 0.00 for non-survivors versus approximately 2.82 for survivors, though these exemplary values may vary. In embodiments, the prognostic value assessor 814 can apply statistical significance testing processing circuits that achieve p-values of less than 0.001.

In embodiments, a population stratification engine 816 within the accuracy measurement system 800 organizes patient data into clinically relevant subgroups. The population stratification engine 816 includes processing circuits that receive metadata associated with each measurement, including patient demographic information, diagnosis codes, and medication administration records. In embodiments, the population stratification engine 816 can implement metadata parsing processing circuits that extract classification variables. The population stratification engine 816 implements severity classification processing circuits that separate measurements from patients with a GCS at or below 8 from those with a GCS above 8. The population stratification engine 816 generates statistical summary data structures showing measurement distributions. In embodiments, the population stratification engine 816 can calculate median pupil reactivity scores such as approximately 1.85 for severe cases and approximately 4.11 for non-severe cases, though these exemplary values may vary. The population stratification engine 816 implements diagnosis-based stratification processing circuits that categorize patients into diagnostic groups.

In embodiments, a statistical significance tester 818 within the accuracy measurement system 800 applies hypothesis testing procedures. The statistical significance tester 818 includes processing circuits that receive comparison data structures from other processing stages. In embodiments, the statistical significance tester 818 can implement a library of statistical test processing circuits that support both parametric and non-parametric tests. The statistical significance tester 818 implements t-test processing circuits for comparing mean values and Mann-Whitney U test processing circuits for comparing distributions without normal distribution assumptions. The statistical significance tester 818 calculates p-values by implementing probability distribution processing circuits that evaluate test statistics against theoretical reference distributions. In embodiments, the statistical significance tester 818 can apply multiple comparison correction processing circuits when testing multiple hypotheses simultaneously. The statistical significance tester 818 generates annotation data structures that convey the strength of statistical evidence using symbols that indicate significance levels.

In embodiments, a comparative performance evaluator 820 within the accuracy measurement system 800 benchmarks computational pupillometry techniques against traditional infrared pupillometry systems. The comparative performance evaluator 820 includes processing circuits that receive measurement accuracy data alongside published specifications from some existing devices. In embodiments, the comparative performance evaluator 820 can implement literature retrieval processing circuits that access stored manufacturer specifications and peer-reviewed publications. The comparative performance evaluator 820 generates bar chart visualization data structures showing measurement accuracy across different systems. The comparative performance evaluator 820 creates data structures that show computational approaches achieve approximately 0.025 millimeter accuracy, compared to some of the existing systems that may achieve approximately 0.03 to 0.10 millimeter accuracy, although these exemplary values may vary. In embodiments, the comparative performance evaluator 820 can perform statistical hypothesis testing on processing circuits. The comparative performance evaluator 820 compares light-invariant behavior against light-dependent behavior of traditional parameters, demonstrating that some of the exiting or commonly used metrics vary by approximately 41% to 133% across different lighting conditions.

In embodiments, a longitudinal trend analyzer 822 within the accuracy measurement system 800 processes time-series pupillometry data to identify patterns. The longitudinal trend analyzer 822 includes processing circuits that receive timestamped pupil reactivity score sequences. In embodiments, the longitudinal trend analyzer 822 can implement temporal data organization processing circuits that sort measurements chronologically and calculate time intervals between successive measurements. The longitudinal trend analyzer 822 employs smoothing processing circuits, including moving average processing circuits and spline fitting processing circuits, which construct piecewise polynomial functions. The longitudinal trend analyzer 822 generates graphical visualization data structures, showing pupil reactivity score trajectories plotted against time. In embodiments, the longitudinal trend analyzer 822 can create annotation data structures marking critical clinical events. The longitudinal trend analyzer 822 implements change-point detection processing circuits that apply statistical techniques to identify times when data-generating processes change. In embodiments, the longitudinal trend analyzer 822 can integrate pupillometry trends with parallel trajectories of other clinical variables, including vital signs, laboratory values, and neurological assessments.

In embodiments, the accuracy measurement system 800 operates across the full clinical validation workflow. The accuracy measurement system 800 implements data aggregation processing circuits that collect measurements across multiple patients, measurement sessions, and healthcare facilities. In embodiments, the accuracy measurement system 800 can process clinical study data from neurocritical care settings where patients present with diverse neurological conditions, including hemorrhagic stroke, traumatic brain injury, hydrocephalus, brain tumors, and intracranial hypertension. The accuracy measurement system 800 demonstrates that computational pupillometry techniques achieve measurement precision of approximately ±0.025 millimeters across all iris colors, validating light invariance across ambient conditions ranging from approximately 4 lux to 1200 lux (and in some embodiments across about 0.1 lux to about 3000 lux), with absolute Spearman rank correlation coefficients below approximately 0.15 between PuRe and log₁₀(lux). In embodiments, the accuracy measurement system 800 can confirm clinical correlation, achieving Spearman correlation coefficients of at least 0.7, establish diagnostic accuracy with ROC AUC values exceeding 0.9, identify optimal thresholds achieving approximately 84.8% sensitivity and approximately 90.0% specificity, and validate prognostic utility with p-values less than 0.001. However, all these exemplary values vary across populations. The accuracy measurement system 800 generates standardized reports that present validation results in formats suitable for clinical decision-making, regulatory submissions, and scientific publication.

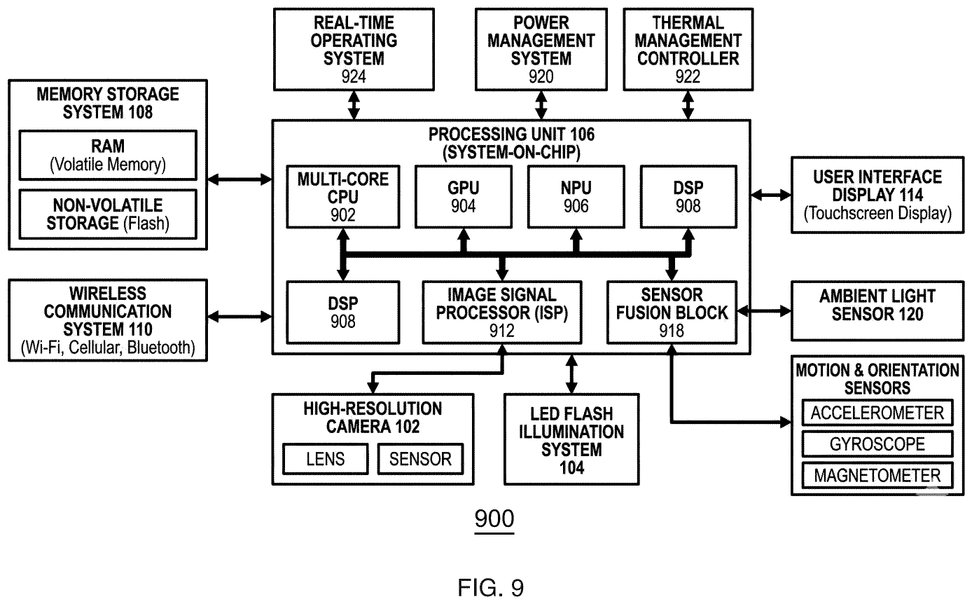

Regarding FIG. 9, a smartphone hardware platform 900 provides an integrated processing infrastructure that enables real-time computational pupillometry with light-invariant measurement capabilities through a distributed, heterogeneous computing architecture that combines specialized processing circuits, imaging sensors, adaptive illumination systems, and communication subsystems.

In embodiments, as indicated by the arrows in FIG. 9, image data captured by a camera sensor 102 are processed by an image signal processor 912 and stored in a memory 108 for access by processing elements including a multi-core central processing unit (CPU) architecture 902, a graphics processing unit (GPU) 904, and a neural processing unit (NPU) 908 to compute pupillometry parameters, ambient illumination estimates, quality metrics, and one or more scores. In embodiments, the CPU architecture 902 may coordinate timing and control an adaptive flash control system 104, and results, data, and images generated and output by the processing unit 106 may be rendered and/or displayed via a user interface 114 (e.g., a display of the user interface 114) and/or transmitted via a wireless communication system (e.g., a modem) 110.

In embodiments, a multi-core CPU architecture 902 executes deterministic mathematical processing operations for pupillometry score calculations within the smartphone hardware platform 900. The multi-core CPU architecture 902 comprises multiple independent processor cores that perform arithmetic operations, including exponential evaluations required for saturation correction processing. The multi-core CPU architecture 902 applies saturation correction techniques, incorporating exponential transformations that utilize pupillary parameters, including constriction amplitudes, constriction velocities, and dilation velocities, along with ambient light measurements, to generate light-invariant scores. The multi-core CPU architecture 902 executes timestamp synchronization between video frames and flash activation events to enable precise temporal alignment, which is necessary for accurate light level estimation through pre-flash and post-flash frame comparisons.

In embodiments, a graphics processing unit 904 provides massively parallel processing capabilities within the smartphone hardware platform 900. The graphics processing unit 904 comprises processing cores that execute identical instructions on multiple data elements simultaneously, performing pixel-level operations across entire video frames in parallel. The graphics processing unit 904 calculates statistical aggregations, including mean, median, and root-mean-square intensity values, extracted from frames before and after flash transitions. This generates compact statistical representations that subsequent machine learning models process to predict ambient light levels for light-invariant score computation. The graphics processing unit 904 implements convolution operations that apply spatial filters for noise reduction and edge enhancement, and executes morphological operations on segmented pupil masks to refine boundaries.

In embodiments, a neural processing unit 908 provides specialized hardware acceleration for artificial intelligence inference operations within the smartphone hardware platform 900. The neural processing unit 908 comprises dedicated circuit blocks designed for executing neural network computations through matrix multiplication engines that perform multiply-accumulate operations across arrays of numerical values representing neural network weights and input activations. The neural processing unit 908 executes deep learning models for pupil detection and segmentation, processing video frame data through multiple convolutional layers that extract hierarchical feature representations. Early layers detect edges and texture patterns, while deeper layers identify iris boundaries and pupil contours. The neural processing unit 910 performs inference operations with minimal latency, completing the processing of individual video frames within milliseconds to enable real-time feedback during pupillometry measurements. However, actual processing times vary depending on the complexity of the neural network architecture and hardware specifications.

In embodiments, a high-resolution camera sensor 102 captures optical imagery within the smartphone hardware platform 900. The high-resolution camera sensor 102 comprises a two-dimensional array of photodetector elements that convert incident photons into electrical charge, integrating charge over controlled exposure durations and transferring accumulated charge through readout circuitry that converts charge quantities into digital values representing pixel intensities. The high-resolution camera sensor 102 captures video sequences at frame rates ranging from 30 to 240 frames per second, with 60 frames per second representing a typical operating point; however, these values are examples, and actual rates may vary. The high-resolution camera sensor 102 synchronizes frame capture timing with illumination events from the adaptive flash control system 104, enabling the systematic capture of pre-flash frames before illumination activates and post-flash frames after illumination reaches its steady-state intensity, for accurate temporal alignment between captured imagery and illumination states.

In embodiments, an adaptive flash control system 104 manages the delivery of illumination within the smartphone hardware platform 900. The adaptive flash control system 104 comprises light-emitting elements that generate visible-spectrum illumination for stimulating the pupillary light reflex. It includes driver circuits that control the electrical current flowing through the light-emitting elements, modulating the intensity of the light output. The adaptive flash control system 104 adjusts illumination intensity based on measurement distance by receiving distance estimates and applying inverse-square law corrections. It adjusts based on ambient light conditions by receiving estimates of ambient light levels and increasing the baseline illumination intensity in brighter environments, where larger light increments elicit measurable pupillary responses, while reducing intensity in darker environments. The adaptive flash control system 104 implements precise temporal control over illumination delivery, activating light emission at specific timestamps synchronized with video frame capture.

In embodiments, an image signal processor 912 performs initial processing operations on image data within the smartphone hardware platform 900. The image signal processor 9124 comprises dedicated hardware circuits that execute processing pipelines on raw sensor data from the high-resolution camera sensor 102, implementing black level correction, defect pixel correction, and lens shading correction. The image signal processor 912 executes demosaicing operations that convert color filter array data into full-color images, applies color correction matrices, performs white balance adjustments, and implements gamma correction. The image signal processor 912 implements exposure control feedback loops by analyzing captured frame statistics and generating control signals that adjust camera sensor parameters for subsequent frames.

In embodiments, a memory hierarchy 108 provides data storage within the smartphone hardware platform 900. The memory hierarchy 108 comprises volatile random-access memory, providing fast read and write operations, and non-volatile storage, providing persistent data retention across power cycles. The memory hierarchy 108 allocates buffer space for video frame sequences captured during pupillometry measurements, stores neural network model parameters, including weight matrices and bias vectors that define trained neural network architectures, and maintains pupillometry measurement results, patient identification data, and associated metadata in local databases that enable offline operation when network connectivity to cloud infrastructure is unavailable. The memory hierarchy 108 implements encryption for protected health information, applying encryption techniques before writing sensitive data to non-volatile storage.

In embodiments, a wireless communication system 110 may enable data transmission within the smartphone hardware platform 900. In some cases, the wireless communication system 110 may comprise one or more wireless communication chips. In some embodiments, the wireless communication system 110 may comprise one or more radio frequency transceivers that implement multiple wireless protocols, including cellular communication standards, WiFi standards, Bluetooth standards, and near-field communication standards. The wireless communication system 110 transmit pupillometry measurement data to remote servers, establish connections to the backend cloud infrastructure through available networks, implement transport protocols that ensure reliable data delivery, and coordinate with encryption subsystems to secure the transmitted data, protecting patient health information. The wireless communication system 110 communicate with electronic medical record systems, transmitting measurement data in standardized healthcare data formats, including Health Level Seven Fast Healthcare Interoperability Resources representations.

In embodiments, a sensor fusion block 918 integrates data from multiple sensing elements within the smartphone hardware platform 900. The sensor fusion block 918 comprises processing circuits that receive data streams from accelerometers measuring linear acceleration, gyroscopes measuring rotational rates, and magnetometers measuring magnetic field orientation. These circuits implement filtering techniques that combine data from multiple sensors to produce estimates with lower noise and higher accuracy. The sensor fusion block 918 monitors accelerometer and gyroscope readings during measurement periods to detect excessive hand motion that might compromise image stability, calculates motion magnitude metrics, provides these metrics to quality validation systems, and triggers warnings when detected motion exceeds predetermined thresholds. The sensor fusion block 918 enables motion compensation during image analysis by providing motion state estimates that correlate with captured video frames.

In embodiments, a power management system 920 regulates energy delivery within the smartphone hardware platform 900. The power management system 920 comprises voltage regulators that convert the battery voltage to multiple supply voltages required by different subsystems and implements dynamic voltage and frequency scaling by adjusting the supply voltages and clock frequencies delivered to processing elements based on the computational workload. The power management system 920 provides current-limited supplies to light-emitting elements within the adaptive flash control system 104 and coordinates with the thermal management controller 922 to prevent overheating by implementing thermal throttling policies that reduce power delivery when temperatures approach safety limits.

In embodiments, a thermal management controller 922 regulates temperature within the smartphone hardware platform 900. The thermal management controller 922 comprises temperature sensors positioned at thermally critical locations and includes control logic circuits that read temperature sensor values and implement thermal mitigation strategies. The thermal management controller 922 interfaces with the power management system 920 to reduce energy delivery to heat-generating elements when temperatures exceed predetermined thresholds. It coordinates with task schedulers to postpone or throttle non-critical processing when thermal conditions are marginal and implements duty cycling for intensive operations. The thermal management controller 922 implements flash duty cycle limits, preventing excessive heating of light-emitting elements within the adaptive flash control system 912 during repeated measurements.

In embodiments, a real-time operating system 924 manages software execution within the smartphone hardware platform 900. The real-time operating system 924 comprises kernel software executing on the multi-core CPU architecture 906, which provides services including process scheduling, memory management, inter-process communication, and device driver interfaces. This implementation supports preemptive multitasking and priority-based scheduling, where higher-priority tasks preempt lower-priority tasks. The real-time operating system 924 schedules camera control processes that configure the high-resolution camera sensor 902 and initiate frame capture, schedules flash control processes that command the adaptive flash control system 912 to activate illumination at precise timestamps, and schedules processing tasks that transfer captured frames to the graphics processing unit 908 or neural processing unit 910 for analysis. The real-time operating system 924 assigns high priority to processes handling frame capture and flash control to ensure these processes receive processing resources within bounded time intervals, thereby ensuring accurate timing coordination.

In embodiments, a hardware acceleration engine 926 provides specialized processing capabilities within the smartphone hardware platform 900. The hardware acceleration engine 926 comprises dedicated circuit blocks that implement specific computational operations with higher performance or energy efficiency than can be achieved through software execution on general-purpose processors. The hardware acceleration engine 926 implements hardware-based video scaling circuits that resample frames to different resolutions, perform format conversion operations transforming between different color representations, and accelerate Advanced Encryption Standard operations that encrypt protected health information before storage in the memory hierarchy 916 or transmission through the wireless communication system 110. The hardware acceleration engine 926 includes floating-point arithmetic circuits that perform exponential, logarithmic, and trigonometric operations with hardware-level efficiency for pupillometry calculations.

The smartphone hardware platform 900 enables computational pupillometry through coordinated operation of its heterogeneous processing elements. The distributed processing architecture executes neural network inference on the neural processing unit 910 for pupil detection and segmentation, mathematical calculations on the multi-core CPU architecture 906 for score computation using saturation correction techniques incorporating pupillary parameters and ambient light measurements, parallel image operations on the graphics processing unit 908 for frame analysis including statistical feature extraction used for ambient light estimation, and specialized operations on the hardware acceleration engine 926 for video processing and cryptographic protection. The sensor fusion block 918 provides motion detection for quality validation. The power management system 920 and thermal management controller 922 maintain stable and efficient operation. The real-time operating system 924 coordinates timing-critical operations, including flash activation synchronized with frame capture. The system processes pupillometry videos, achieving pupil diameter measurement accuracy of approximately ±0.025 millimeters, as one validated implementation example; however, actual accuracy varies based on measurement conditions. The platform 900 supports real-time processing with a frame-to-frame latency of less than about 250 milliseconds in typical implementations, although actual latency may vary. The platform 900 enables offline operation with local data storage, followed by cloud synchronization when connectivity is restored. It integrates with electronic medical records through standardized healthcare data protocols and maintains regulatory-compliant security for protected health information through encryption, access controls, and audit logging.

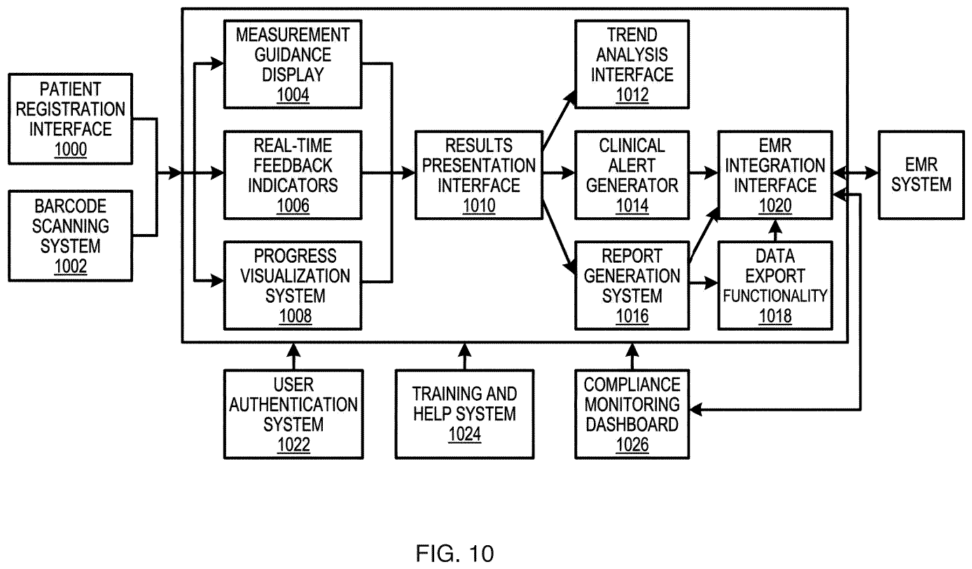

FIG. 10 is a block diagram of a clinical user interface and workflow integration system. In some embodiments, the processing unit 106 of the computing device 100 may execute machine readable instructions to implement the clinical user interface and workflow integration system shown in FIG. 10. In some embodiments, the clinical user interface and workflow integration system may be configured to authenticate and register a user and allow a registered and/or authenticated user to enter data, request a report, observe and analyze pupillometry results, observe alerts, or otherwise interact with the computing system 100. In some embodiments, the clinical user interface and workflow integration system may communicate with an EMR system (e.g., the EMR system 126 described above with respect to FIG. 1) to send and receive patient data (e.g., pupillometry results and reports).

In some embodiments, the clinical user interface and workflow integration system may comprise patient registration interface 1000 that can be configured to render graphical input elements (e.g., on the user interface display 114 to capture patient identification information before a pupillometry measurement session. In embodiments, the patient registration interface 1000 can display text entry fields for alphanumeric patient identifiers, dropdown selection menus for demographic data, and touch-responsive buttons that initiate registration workflows. The patient registration interface 1000 validates entered data against predefined formatting rules and stores patient information for subsequent measurement sessions.

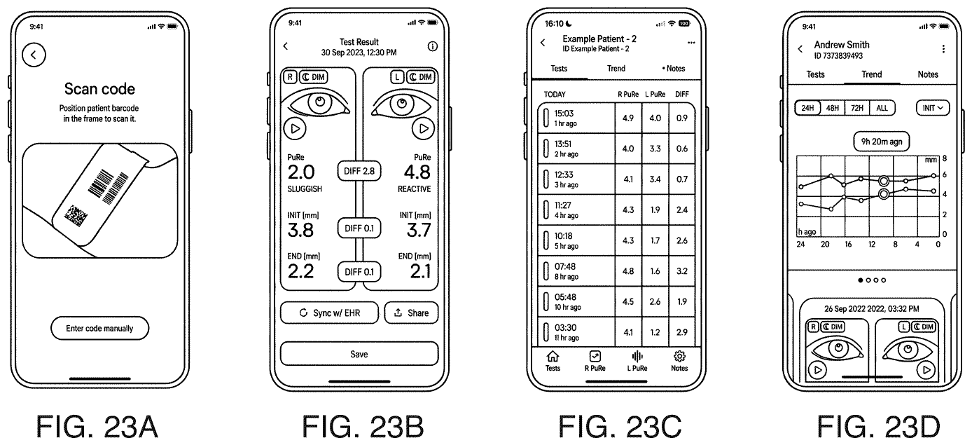

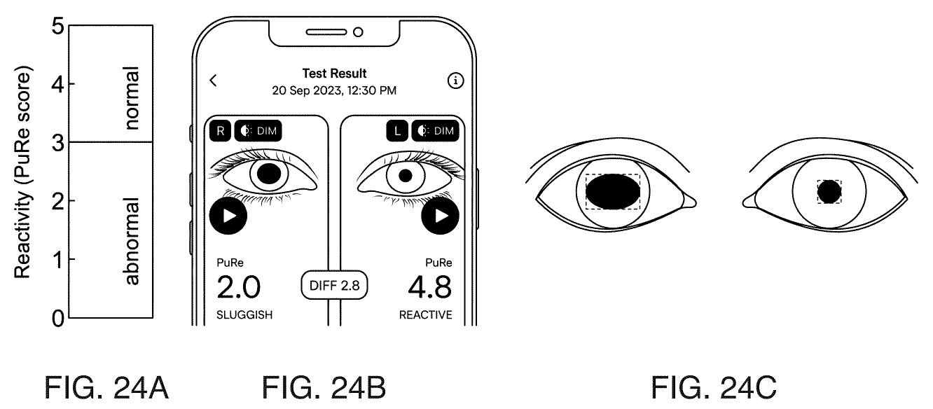

In embodiments, as indicated by the arrows in FIG. 10, patient identifiers captured by the patient registration interface 1000 are provided to downstream workflow screens, and live capture guidance is provided by a measurement guidance display 1004 and real-time feedback indicators 1006 that receive sensor and video analytics. In embodiments, a capture trigger (e.g., in the process visualization system) 1008 initiates recording and scoring, results are rendered by a results presentation interface 1010 and a trend analysis interface 1012, and a clinical alert generator 1014 produces alerts when one or more thresholds are satisfied. In embodiments, exemplary user interface screens for patient registration, results presentation, patient history review, and trend visualization are shown in FIGS. 23A-23D. FIGS. 23A-23D are diagrams illustrating example user interface screens for patient registration, measurement presentation, patient history review, and longitudinal trend visualization, in accordance with embodiments of the present disclosure. An anisocoria example with differential reactivity between eyes is shown in FIGS. 24A-24C. FIGS. 24A-24C are diagrams illustrating example normal and abnormal ranges for pupil reactivity score (FIG. 24A), and example test results (FIG. 24B) for an eye with anisocoria, abnormal pupil shape (FIG. 24C), depicting differential pupillary reactivity between eyes, in accordance with embodiments of the present disclosure.

In embodiments, a barcode scanning system 1002 activates imaging capture optimized for decoding barcode patterns on patient wristbands. The barcode scanning system 1002 renders a rectangular target region that guides barcode positioning within the field of view. In embodiments, the barcode scanning system 1002 can execute edge detection operations to identify barcode structures and employ decoding circuits that convert detected patterns into alphanumeric character strings representing patient identifiers. The barcode scanning system 1002 validates decoded information against checksum digits and generates auditory confirmation tones when successful capture occurs.

In embodiments, a measurement guidance display 1004 renders visual instructions and dynamic feedback that direct healthcare providers through proper positioning during pupillometry assessments. The measurement guidance display 1004 displays graphical representations showing optimal distances and angles for measurements. In embodiments, the measurement guidance display 1004 can overlay semi-transparent target regions on live preview images, delineating zones where proper eye positioning should occur. The measurement guidance display 1004 analyzes real-time video streams, calculating spatial relationships between detected facial landmarks and generating positioning metrics. In embodiments, the measurement guidance display 1004 can render directional arrows and textual instructions specifying corrective movements. The measurement guidance display 1004 renders color-coded status indicators, utilizing green elements for optimal conditions, yellow elements for suboptimal but acceptable conditions, and red elements for conditions that prevent measurement initiation.

In embodiments, real-time feedback indicators 1006 generate dynamic visual and auditory signals that convey instantaneous quality assessments during measurement phases. The real-time feedback indicators 1006 receive data streams from motion sensors, ambient light sensors, and image analysis circuits, synthesizing these inputs to produce quality metrics. In embodiments, the real-time feedback indicators 1006 can display stability measurements derived from accelerometer data, rendering graphical elements that visualize the magnitude of motion. The real-time feedback indicators 1006 evaluate detected eye positions, calculating confidence scores that quantify the reliability of pupil boundary detection, and translate these scores into visual representations comprising color-coded circles, bars, or numerical percentage displays. In embodiments, the real-time feedback indicators 1006 can monitor ambient light levels, comparing measurements against acceptable ranges and generating warning indicators when illumination deviates from optimal ranges.

In embodiments, a progress visualization system 1008 renders sequential workflow representations that depict the current stage within multi-step measurement sequences. The progress visualization system 1008 accesses workflow state information, retrieves stage identifiers, and calculates completion percentages. In embodiments, the progress visualization system 1008 can display horizontal progress bars that fill incrementally as successive stages are completed, including patient registration, quality validation, video capture, pupil detection, parameter extraction, and score calculation. The progress visualization system 1008 renders numbered step indicators, highlighting the active step with distinct visual styling. In embodiments, the progress visualization system 1008 can present circular or radial progress indicators that sweep through angular ranges representing the completion of measurements.

In embodiments, a results presentation interface 1010 arranges measurement outcomes and calculated parameters in structured layouts, organizing pupillometry data for clinical interpretation. The results presentation interface 1010 retrieves measurement data, accessing stored pupil reactivity scores, diameter measurements, temporal parameters, and associated metadata. In embodiments, the results presentation interface 1010 can display primary pupil reactivity scores as prominently positioned numerical values, with specific threshold values representing boundaries between normal and abnormal responses, though these parameters serve as examples. The results presentation interface 1010 renders separate result panels for left-eye and right-eye measurements, positioning these panels adjacent to facilitate comparison. In embodiments, the results presentation interface 1010 can apply color coding schemes, displaying higher scores in green hues, intermediate scores in yellow or orange hues, and lower scores in red hues.

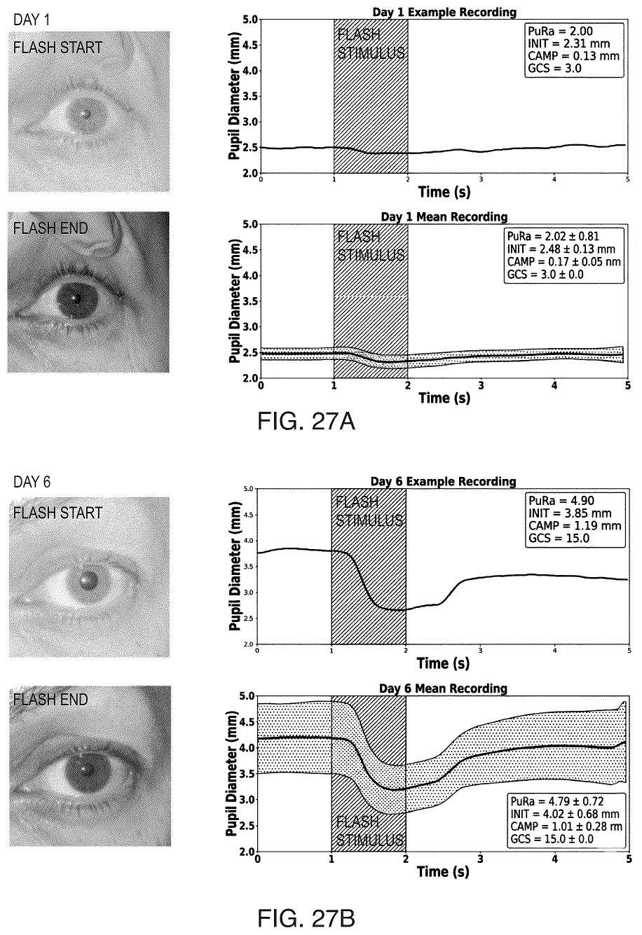

In embodiments, a trend analysis interface 1012 aggregates temporal sequences of pupillometry measurements, visualizing changes in pupillary function across hours, days, or extended periods. The trend analysis interface retrieves 1012 queries from databases to organize historical measurement records in chronological sequence. In embodiments, the trend analysis interface 1012 can render line graphs plotting pupil reactivity scores as functions of measurement time, connecting sequential data points with line segments that reveal patterns of improvement, deterioration, or stability. The trend analysis interface 1012 applies smoothing techniques to plotted data, reducing noise while preserving clinically significant variations. In embodiments, the trend analysis interface 1012 can overlay reference threshold lines at clinically significant values. Examples of longitudinal monitoring results across multiple days are illustrated in FIGS. 27A-27B. results across multiple days showing patient's stroke recovery, including exemplary frames and pupillograms, in accordance with embodiments of the present disclosure.

In embodiments, a clinical alert generator 1014 analyzes measurement data using computational processing to identify conditions requiring clinical attention and automatically generates notification signals. The clinical alert generator 1014 applies threshold comparison logic to measured pupil reactivity scores, detecting when values fall below predefined critical levels or when asymmetry measurements exceed predefined difference thresholds. However, these threshold values serve as examples. In embodiments, the clinical alert generator 1014 can execute rate-of-change analysis on sequential measurements, calculating temporal derivatives and identifying rapid deterioration patterns. The clinical alert generator 1014 transmits notification signals through secure communication pathways, establishing connections to hospital paging systems or messaging platforms. In embodiments, the clinical alert generator 1014 can display prominent visual alerts utilizing high-contrast color schemes and produce distinctive alert tones with specific frequency patterns.

In embodiments, a report generation system 1016 compiles measurement data, analysis results, and contextual information into formatted documents suitable for medical records. The report generation system 1016 accesses structured measurement data, retrieving parameters associated with completed assessments, including primary scores, temporal parameters, and quality metrics. In embodiments, the report generation system 1016 can apply document templates, populating predefined fields with measurement-specific data while maintaining consistent formatting. The report generation system 1016 executes text formatting operations that arrange data into tables, lists, and paragraphs. In embodiments, the report generation system 1016 can incorporate graphical elements, including trend plots and result visualizations.

In embodiments, data export functionality 1018 serializes measurement data into structured file formats compatible with external analysis tools, research databases, and healthcare information systems. The data export functionality 1018 retrieves selected datasets, applying query logic that filters measurements based on temporal ranges, patient identifiers, or quality criteria. In embodiments, the data export functionality 1018 can generate comma-separated value files that arrange measurement parameters in a tabular format or implement JavaScript Object Notation (JSON) formatting to structure hierarchical data relationships. The data export functionality 1018 applies data anonymization processing when generating research datasets, removing or encrypting patient identification information. In embodiments, the data export functionality 1018 can transmit exported data files through secure transfer protocols that implement encryption during transit.

In embodiments, an EMR integration interface 1020 establishes bidirectional communication pathways with electronic medical record systems, implementing standardized healthcare data exchange protocols. The EMR integration interface 1020 utilizes secure communication channels to transmit measurement results, patient identifiers, and metadata through network connections employing encryption techniques. In embodiments, the EMR integration interface 1020 can implement Health Level Seven Fast Healthcare Interoperability Resources messaging protocols that structure transmitted data according to healthcare data standards. The EMR integration interface 1020 formats pupillometry data into observation resources that represent individual measurements with appropriate coding, units, and timestamps. In embodiments, the EMR integration interface 1020 can query electronic medical record systems to retrieve patient demographic information and clinical notes that provide contextual information.

In embodiments, a user authentication system 1022 implements security controls that restrict access to pupillometry measurement capabilities and patient data to authorized healthcare personnel. The user authentication system 1022 presents login screens that prompt users to enter identification credentials, including username and password combinations or biometric data. In embodiments, the user authentication system 1022 can capture fingerprint patterns using biometric sensors and compare the captured patterns against stored authorized user fingerprints. The user authentication system 1022 implements password hashing techniques that store password representations using one-way cryptographic transformations. In embodiments, the user authentication system 1022 can implement multi-factor authentication requiring users to provide multiple forms of identity verification and role-based access control that associates authenticated users with specific permission sets.

In embodiments, a training and help system 1024 provides instructional content and reference materials accessible through display interfaces that support proper system usage and measurement technique optimization. The training and help system 1024 stores tutorial content, including text-based instructions, still images, video demonstrations, and interactive simulations that teach measurement workflows. In embodiments, the training and help system 1024 can present context-sensitive help information that automatically displays relevant instructional content based on the user's current workflow stage. The training and help system 1024 tracks user interaction patterns, identifying repeated measurement attempts or quality failures that suggest the need for additional training. In embodiments, the training and help system 1024 can include video demonstrations that show proper device and patient positioning, and render interactive tutorials that guide users through simulated measurement sequences.

In embodiments, a compliance monitoring dashboard 1026 aggregates system usage data, quality metrics, and regulatory compliance indicators, supporting institutional oversight and quality assurance review. The compliance monitoring dashboard 1026 queries databases to retrieve comprehensive usage statistics, including the number of measurements performed, success rates, and quality failure rates. In embodiments, the compliance monitoring dashboard 1026 can display temporal trend graphs showing measurement volumes over time. The compliance monitoring dashboard 1026 calculates quality performance metrics, including percentages of measurements meeting predefined quality thresholds and mean quality scores. In embodiments, the compliance monitoring dashboard 1026 can generate comparison displays showing performance metrics across different devices, users, or clinical locations. The compliance monitoring dashboard 1026 presents audit trail information documenting all system access, data modifications, and data transmissions, supporting compliance with healthcare privacy regulations.

The following text includes details of a method(s) or a flow diagram(s) per embodiments of this disclosure. For simplicity of explanation, each method is depicted and described as a set of alterable operations. Additionally, one or more operations can be performed in parallel, concurrently, or in a different sequence. Further, not all the illustrated operations are required to implement each method described by this disclosure.

### Centralized Pupillometry Measurement Management

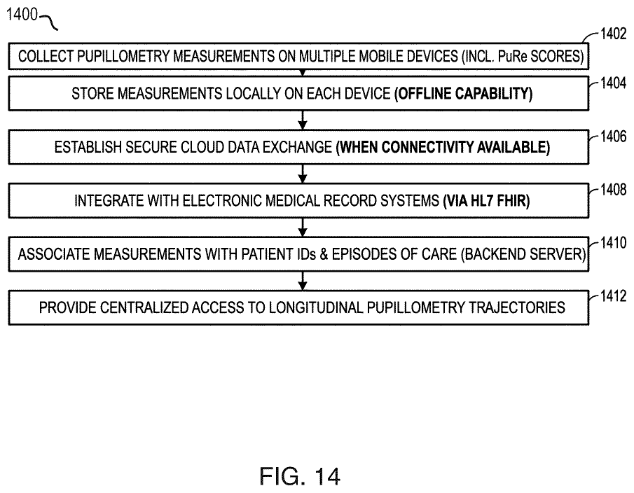

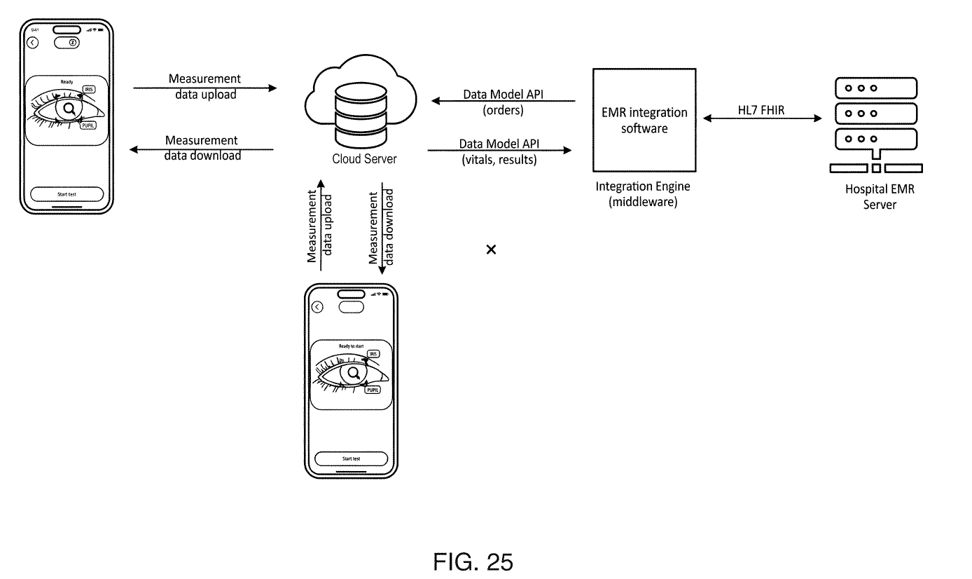

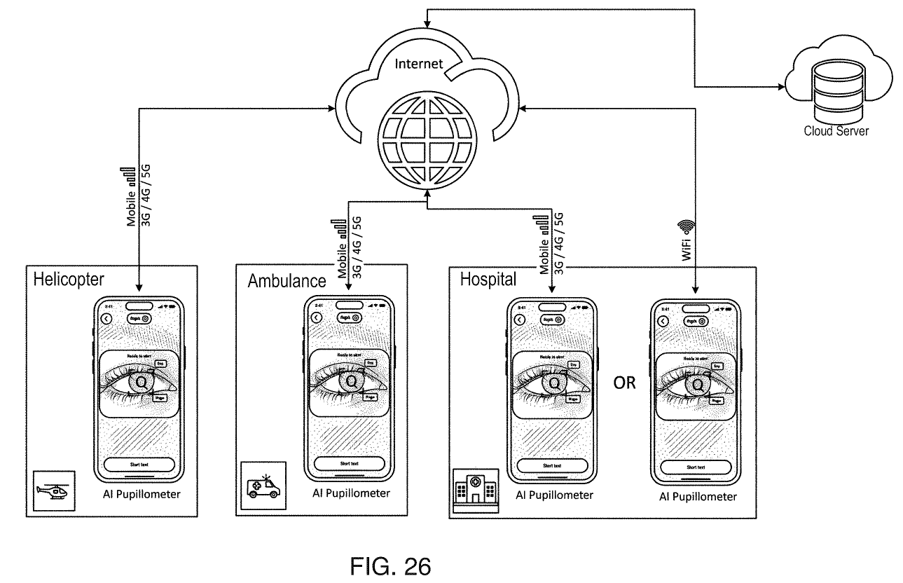

Regarding FIG. 5, a device fleet network 500 operates as a coordination infrastructure that supports synchronization of pupillometry measurements between a pupillometry device and a server such that additional pupillometry devices can access consistent patient records. In embodiments, a first pupillometry device captures a pupillometry measurement, stores the measurement locally, and uploads the measurement to a server through wireless networking protocols. In embodiments, a second pupillometry device retrieves the same patient's synchronized measurement record from the server to support continuity across care settings. In embodiments, the device fleet network 500 includes access control, audit logging, and conflict resolution such that each device presents a consistent view of patient data. In embodiments, an example method flow for such synchronization is described with respect to FIG. 14. In embodiments, example synchronization and deployment diagrams are shown in FIG. 25 and FIG. 26.

In embodiments, as indicated by the arrows in FIG. 5, a measurement record flows from local storage on a first pupillometry device (e.g., a local data storage facility 502) through an offline capability coordinator 504 and a cloud synchronization processor 508 to a server, and then from the server to a second pupillometry device to provide a consistent patient record view. In embodiments, updates (e.g., updated parameters and/or model weights) flow from the server to the pupillometry device(s) through a calibration synchronization process 518 and fleet management controls 520, and conflicts are resolved using a data conflict resolution processor 510.

In embodiments, a local data storage facility 502 can function as an embedded database structure that maintains pupillometry measurements, patient information, and configuration data directly on each mobile device. The local data storage facility 502 employs encrypted database schemas that store video recordings, extracted pupillary parameters, calculated PuRe scores, and associated metadata in a format optimized for mobile device storage constraints. In embodiments, the local data storage facility 502 can utilize SQLite database architecture with custom indexing schemes that enable rapid retrieval of patient records and measurement histories. The local data storage facility 502 implements data compression techniques that reduce storage footprint while maintaining measurement precision, allowing each device to store approximately 10,000 to 50,000 pupillometry recordings, depending on available device memory. The local data storage facility 502 incorporates transaction logging mechanisms that track all data modifications and ensure data integrity even during unexpected interruptions or power failures.

In embodiments, an offline capability coordinator 504 can maintain full pupillometry functionality when network connectivity becomes unavailable or unreliable. The offline capability coordinator 504 implements intelligent caching strategies that preload essential resources, calibration data, and patient information to enable continued operation during network outages. In embodiments, the offline capability coordinator 504 can detect network connectivity loss within 250 milliseconds to 2 seconds and automatically transition the device to offline mode while preserving all measurement capabilities. The offline capability coordinator 504 employs data queuing mechanisms that store new measurements, patient registrations, and events in temporary buffers that automatically synchronize when connectivity resumes.

In embodiments, a network connectivity analyzer 506 can continuously assess the quality and availability of wireless network connections to optimize data transmission and synchronization operations. The network connectivity analyzer 506 implements signal strength assessment techniques that evaluate WiFi signal quality, cellular network performance, and internet connectivity status at regular intervals, typically every 30 seconds to 2 minutes. In embodiments, the network connectivity analyzer 506 can measure network latency, bandwidth availability, and packet loss rates to determine optimal transmission strategies for different types of data.

In embodiments, a cloud synchronization processor 508 can coordinate bidirectional data exchange between a pupillometry device and a centralized server through secure communication protocols. In embodiments, a first pupillometry device transmits only modified or new records (differential synchronization) to the server, reducing bandwidth consumption and improving synchronization efficiency. In embodiments, the server returns one or more updates (e.g., newly created records or corrected records) to the first pupillometry device that originated from another device and/or from an integrated clinical system. In embodiments, synchronization requests are processed using priority-based queuing that prioritizes critical patient data over routine updates or analytics information.

In embodiments, the pupillometry device participates in continual learning and model update workflows for one or more computational models used for ambient illumination estimation and/or PuRe score calculation. In some embodiments, a model used for ambient illumination estimation and scoring can be a pre-trained model. In some cases, real-time result generation uses inference only. In some cases, model retraining or recalibration occurs asynchronously (e.g., over days or weeks), optionally using techniques such as hard-example mining, and updated parameters are later deployed to devices. In embodiments, a first pupillometry device uploads de-identified or access-controlled datasets including ambient illumination estimates, pupillary parameters, computed PuRe scores, and associated metadata to centralized storage for aggregation and analysis. In embodiments, ground-truth labels (e.g., invasive ICP measurements, GCS scores, clinical outcomes, and/or clinician annotations) are received by the first device via user input and/or via integration with an electronic medical record system and are included in an upload to the server. In embodiments, an aggregated analytics and model update process generates updated parameters and/or updated model weights (e.g., for a machine learning light prediction model and/or a PuRe scoring model), and distributes the updates to the first pupillometry device and/or to a second pupillometry device via a calibration synchronization process 518 and fleet management controls 520. In embodiments, the update process includes federated learning, where local model updates are computed on-device and aggregated without transmitting raw video. In embodiments, each pupillometry result is associated with a model identifier and/or version number to support auditability and retrospective analysis.

In embodiments, a data conflict resolution module or processor 510 can automatically identify and resolve inconsistencies that arise when multiple devices modify patient records or configuration data simultaneously. The data conflict resolution processor 510 may implement a process comprising a multi-tier decision framework configured to processes conflicts through one or more of timestamp-based precedence rules, field-level merging strategies, and semantic analysis methods. In embodiments, the data conflict resolution processor 510 can employ timestamp comparison algorithms that examine modification times down to millisecond precision, automatically selecting the most recent valid change when conflicts occur within a 5-second window. For example, when Device A records a PuRe score of 3.2 at timestamp 2024-10-19T14:30:15.234Z and Device B simultaneously records a PuRe score of 3.8 at timestamp 2024-10-19T14:30:15.567Z for the same patient, the processor 510 selects the later timestamp entry while flagging the discrepancy for clinical review. The data conflict resolution processor 510 utilizes field-level merging strategies that combine non-conflicting data elements from multiple sources, such as preserving demographic information from one record while accepting measurement data from another. In specific implementations, the processor 510 applies semantic analysis rules that understand clinical significance hierarchies, automatically prioritizing measurement data over administrative fields, preserving critical vital signs over routine observations, and maintaining medication administration records over scheduling information. The data conflict resolution processor 510 maintains detailed conflict resolution logs that document all automatic decisions, manual interventions, and rejected changes, creating comprehensive audit trails that include original values, resolved values, conflict types, resolution methods, timestamps, and user identifications for regulatory compliance and quality assurance purposes.

In embodiments, an EMR integration processor 512 can facilitate seamless communication between the pupillometry architecture and existing hospital electronic medical record infrastructures through standardized healthcare data exchange protocols. The EMR integration processor 512 implements HL7 FHIR protocol handlers that translate pupillometry data into standardized healthcare message formats compatible with diverse EMR platforms. In embodiments, the EMR integration processor 512 can establish secure API connections that authenticate with hospital information technology infrastructures using OAuth 2.0 or similar authentication protocols while maintaining HIPAA compliance throughout all data exchanges.

In embodiments, an HL7 FHIR protocol handler 514 can process healthcare data exchange messages according to Fast Healthcare Interoperability Resources standards, enabling compatibility with modern EMR infrastructures and healthcare information exchanges. The HL7 FHIR protocol handler 514 implements resource creation and modification operations that package pupillometry measurements into standardized FHIR observation resources with appropriate clinical context and metadata. In embodiments, the HL7 FHIR protocol handler 514 can generate FHIR-compliant patient resources, encounter resources, and diagnostic report resources that integrate pupillometry results with broader clinical documentation workflows.

In embodiments, a patient record linkage processor 516 can establish and maintain accurate associations between pupillometry measurements and specific patient identities across multiple healthcare organizations and device networks. The patient record linkage processor 516 implements probabilistic matching techniques that compare patient identifiers, demographic information, and clinical context to resolve potential duplicate records or identity conflicts. In embodiments, the patient record linkage processor 516 can utilize master patient index integration that queries centralized patient databases to ensure consistent patient identification across different healthcare facilities and departments.

In embodiments, an audit trail generator 518 can create comprehensive logs of all activities, data modifications, and user interactions to support regulatory compliance, quality assurance, and security monitoring requirements. The audit trail generator 518 implements immutable logging mechanisms that prevent modification or deletion of audit records while maintaining detailed timestamps, user identifications, and action descriptions. In embodiments, the audit trail generator 518 can generate audit logs that comply with healthcare regulations such as HIPAA, FDA requirements for medical device software, and hospital accreditation standards.

In embodiments, a role-based access controller 520 can enforce granular permissions and authentication requirements that restrict access based on user roles, responsibilities, and organizational policies. The role-based access controller 520 implements multi-factor authentication mechanisms that require combinations of passwords, biometric verification, or hardware tokens to ensure secure access to sensitive patient data and functionality. In embodiments, the role-based access controller 520 can define hierarchical permission structures that grant different levels of access to physicians, nurses, technicians, administrators, and research personnel based on their clinical responsibilities and data access requirements.

In embodiments, a data encryption facility 522 can protect sensitive patient information and configuration data through advanced cryptographic techniques that ensure confidentiality during storage and transmission operations. The data encryption facility 522 implements AES-256 encryption standards for data at rest, securing all stored pupillometry recordings, patient information, and configuration data with cryptographic keys managed through secure key management infrastructures. In embodiments, the data encryption facility 522 can employ TLS 1.3 or higher encryption protocols for all network communications, ensuring that data transmission between devices, cloud services, and EMR infrastructures remains protected from interception or tampering.

In embodiments, a backup and recovery processor 524 can maintain data integrity and availability through automated backup procedures and disaster recovery capabilities that protect against data loss from hardware failures, security incidents, or natural disasters. The backup and recovery processor 524 implements incremental backup strategies that capture data changes at regular intervals, typically every 6 to 24 hours, while maintaining multiple backup generations to enable point-in-time recovery operations. In embodiments, the backup and recovery processor 524 can replicate critical data across geographically distributed data centers to ensure availability during regional outages.

In embodiments, a population health analytics processor 526 can process aggregated pupillometry data from across the device fleet to generate insights about patient populations, treatment effectiveness, and clinical trends while maintaining patient privacy through appropriate de-identification techniques. The population health analytics processor 526 implements statistical analysis methods that identify correlations between pupillary measurements and clinical outcomes, medication effects, or demographic factors across large patient cohorts. In embodiments, the population health analytics processor 526 can generate predictive models that utilize machine learning techniques to identify patients at risk for neurological deterioration based on pupillometry trends and clinical context.

### Example Processes

In embodiments, FIG. 18 illustrates an end-to-end sequence of operation. In embodiments, the system captures frames 1800, performs a quality gate 1802, estimates ambient illumination 1804, measures pupillary parameters 1806, computes a normalized score 1808, and outputs and/or synchronizes results 1810. In embodiments, individual subsystem details are described with respect to FIG. 1 (device architecture), FIG. 2 (scoring pipeline), FIG. 4 (quality control), and FIG. 5 (synchronization).

FIG. 11 is a flow diagram of a method 1100 of calculating a normalized pupil reactivity PuRe score using a sequence of video frames. In some cases, the normalized PuRe score may be less sensitive to ambient illumination compared to some of the existing pupillometry systems that do not take into account ambient illumination in PuRe score calculation. In some cases, the method 1100 may normalize pupillometry measurements across varying environmental lighting conditions to ensure consistent pupil reactivity scoring regardless of ambient illumination levels. In some embodiments, the method 1100 may be performed by the processing unit 106 of the computing device 100 based on video frames captured by the imaging device 102.

In some embodiments, the method 1100, at 1102, can include capturing a sequence of video frames of a subject's eye using a mobile device camera. Additionally, at 1104, the method 1100 can include analyzing video frames captured before and after controlled light stimulation to extract statistical intensity features. Further, the method 1100, at 1106, can include processing the extracted features through a computational model to estimate ambient light conditions. At 1108, the method 1100 can also include measuring pupillary response parameters from the video sequence. At 1110, the method 1100 can include calculating a normalized pupil reactivity score using a saturation function that incorporates both the pupillary response parameters and the estimated ambient light conditions to provide consistent scoring across varying environmental lighting.

Further, each operation can include any combination of techniques implemented by the embodiments described herein.

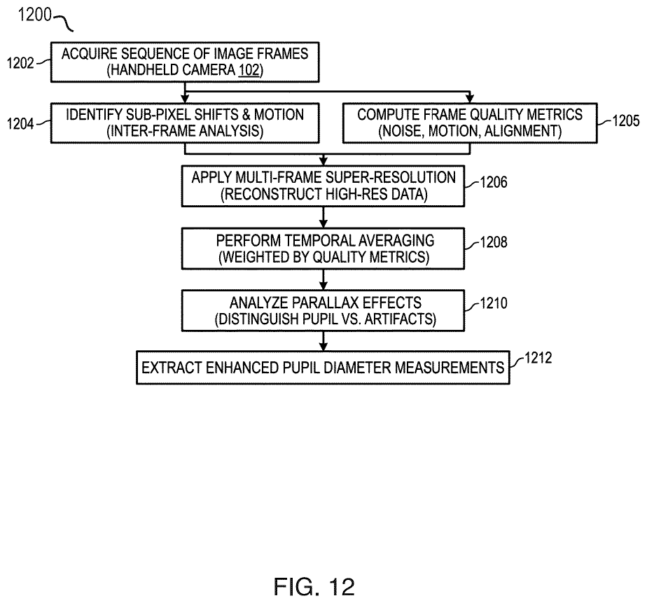

FIG. 12 is a flow diagram of a method 1200 of enhancing precision of certain pupillometry measurements and results through multi-frame processing and integration. In some cases, the method 1200 may leverage natural micro-movement detection and advanced image reconstruction algorithms to overcome single-frame resolution and noise limitations. In some cases, the method 1200 may be performed by the processing unit 106 of the computing device 100 based on video frames captured by the imaging device 102.

For example, the method 1200, at 1202, can include acquiring a sequence of image frames of a subject's eye using a handheld imaging device. Additionally, at 1204, the method 1200 can include identifying sub-pixel shifts between successive frames caused by natural micro-movements. At 1205, the method 1200 can include computing frame quality metrics including noise levels, motion magnitude, and alignment accuracy to weight frames appropriately during subsequent fusion. Further, the method 1200, at 1206, can include applying multi-frame super-resolution algorithms to the sequence to reconstruct higher-resolution pupil boundary information. At 1208, the method 1200 can also include performing temporal averaging across aligned frames to reduce measurement noise. At 1210, the method 1200 can include utilizing parallax effects from micro-movements to distinguish pupil features from artifacts. At 1212, the method 1200 can include extracting pupil diameter measurements with enhanced precision from the processed frame sequence.

Further, each operation can include any combination of techniques implemented by the embodiments described herein.

FIG. 13, is a flow diagram of a method 1300 for monitoring, improving, and evaluating quality and reliability of pupillometry measurement and results. In some case, the method 1300 may comprise evaluating and improving quality of pupillometry measurements and results through comprehensive validation, real-time monitoring, and post-acquisition analysis processes that may verify optimal recording conditions and measurement integrity. In some cases, the method 1300 may be performed by the processing unit 106 of the computing system 100.

For example, the method 1300, at 1302, can include performing pre-recording validation including eye detection confidence assessment, optimal positioning verification, and environmental condition monitoring. Additionally, at 1304, the method 1300 can include initiating pupillary response recording only upon satisfactory validation results. Further, the method 1300, at 1306, can include conducting real-time quality monitoring during data acquisition with feedback provision. At 1308, the method 1300 can also include executing post-recording analysis, including measurement validity verification and artifact detection. At 1310, the method 1300 can include generating quality scores based on multiple assessment criteria. At 1312, the method 1300 can include providing quality-assured pupillometry results with associated confidence metrics.

Further, each operation can include any combination of techniques implemented by the embodiments described herein.

FIG. 14, is a diagram of a method 1400 for synchronizing pupillometry measurement data across multiple devices such that data, improvements, updates, and measurement records generate by a first device are available to and can be used by a second device. In some cases, the method may maintain patient record consistency and enable seamless integration with a electronic medical record systems. In some cases, the method 1400 may be performed at least partially by a sever or a computing system that can be in communication with the two or more computing systems configured to perform pupillometry (e.g., the computing system 100). In some cases, at least one of the computing systems may comprise a mobile computing system (e.g., a smart phone, a tablet or the like).

For example, the method 1400, at 1402, can include collecting pupillometry measurements by a first pupillometry device and storing the measurements locally. Additionally, at 1404, the method 1400 can include maintaining offline capability on the first pupillometry device by queuing measurements and related events for later upload. Further, the method 1400, at 1406, can include establishing secure cloud-based synchronization in which the first pupillometry device uploads one or more measurement records to a server when connectivity is available and downloads one or more records created by a second pupillometry device for the same patient. At 1408, the method 1400 can also include integrating with an electronic medical record system through standardized healthcare data protocols. At 1410, the method 1400 can include maintaining patient record consistency by resolving conflicts and storing audit logs. At 1412, the method 1400 can include providing authorized access to synchronized pupillometry data for healthcare providers via at least one pupillometry device and/or a server.

Further, each operation can include any combination of techniques implemented by the embodiments described herein.

Regarding FIG. 15, a method 1500 relates to correcting pupillometry measurements for confounding factors including age, medications, and physiological variations to isolate neurologically-relevant pupillary changes from non-neurological influences.

For example, the method 1500, at 1502, can include acquiring pupillary response measurements from a subject. Additionally, at 1504, the method 1500 can include collecting demographic information including age and gender data. At 1505, the method 1500 can include obtaining medication history data including active medication lists relevant to pupillary function. Further, the method 1500, at 1506, can include applying age-based normalization using regression models to account for age-related pupillary changes. At 1508, the method 1500 can also include consulting pharmacological databases to determine medication impacts on pupillary responses. At 1510, the method 1500 can include determining iris pigmentation adjustment factors based on iris color. At 1512, the method 1500 can include executing a multi-parameter correction engine with integrated algorithms. At 1514, the method 1500 can include generating corrected neurological-specific pupillary parameters that isolate neurologically-relevant pupillary changes from physiological and pharmacological influences.

In various implementations, computer vision techniques and methods used to extract or compute a parameter (e.g., an eye movement metric) may comprise: an object detection method or model, a segmentation method or model, an open-source model (e.g., “Segment Anything” from Meta), a vision transformer, a Convolutional Neural Network (CNN), or other methods, algorithms, and models. In some cases, the object Detection model may comprise YOLO that can be used to detect bounding boxes for the iris and pupil. In some cases, the object Detection model may be the trained using dedicated datasets. In some cases, a Segmentation model may comprise U-Net that can be used for pixel-wise segmentation of the eye features. In some cases, a vision transformers may comprise a transformer-based model or an open-source model (e.g., “Segment Anything” from Meta). In some cases, a Convolutional Neural Network may comprise an architectures like ResNet or AlexNet that be used to detect key points or directly estimate the diameters of different objects. It should be understood, that the computer vision models are not limited to a single architecture or models and multiple models can be utilized, e.g., in combination. In some cases, these models can include both pre-trained and custom-trained models. In some embodiments, one or more computer vision methods and/models may use deep learning methods, e.g., to ensure generalizability. In some cases, some other computer vision algorithms (e.g., thresholding or Hough transforms), which do not rely on deep learning methods, can be used at least for computing some of the parameters, in some cases, in conjunction with deep learning methods.

In some embodiments, determined ambient light levels may be used to control a light stimulus provide to the eye. For example, in some cases, the system may increase the level light stimulus based on a previously measured or estimated ambient light level. For example, when level light during a pre-stimulus period is high (e.g., bright environments above 300 lux) the stimulus may be increases accordingly.

In some embodiments, one or more computer models used by the system can be pre-trained modules.

As shown in FIG. 13 different modules of the quality control system (shown in FIG. 4) may be used during phase 1, 2, and 3. For example quality control may be used during phase 1 to make a decision to proceed to phase 2, during phase 2 to continue and resume the process, and during phase 3 to generate a confidence score for the results. In some embodiments, the quality control may be used in on or two of these phases. For example in some cases, quality control during phase 2 may be skipped. However, utilizing quality control in all phases may provide more robust results. In some embodiments, quality scores may be calculated primarily in Phase 3.

### EXAMPLE EMBODIMENTS

Various additional example embodiments of the disclosure can be described by the following examples:

Clause 1. A pupillometry system, comprising: an imaging device configured to capture a sequence of video frames of an eye of a subject at least during a stimulus period when the eye is illuminated by a light stimulus; a light source configured to emit the light stimulus toward the eye; a user interface; and at least one processor and a memory storing instructions that, when executed by the at least one processor, cause the at least one processor to: determine a level of ambient illumination associated with the sequence of video frames using one or more of a sensor signal, at least one video frame in the sequence video frames, and a camera exposure setting; determine one or more pupillary response parameters based on the sequence of video frames; compute, based on the level of ambient illumination and the one or more pupillary response parameters, a normalized pupil reactivity score; and transmit the normalized pupil reactivity score to one or both the user interface and an external system separated from the pupillometry system.

Clause 2. The pupillometry system of clause 1, wherein the imaging device is further configured to capture at least one video frame before onset of the stimulus period.

Clause 3. The pupillometry system of clause 1, wherein the imaging device is further configured to capture at least one video frame after termination of the stimulus period.

Clause 4. The pupillometry system of clause 1, further comprising a light sensor configured to generate the sensor signal indicative of the level of ambient illumination.

Clause 5. The pupillometry system of clause 1, wherein executing the instructions causes the at least one processor to determine the level of ambient illumination by implementing a computational model configured to estimate the level of ambient illumination based on the at least one video frame.

Clause 6. The pupillometry system of clause 5, wherein estimating the level of ambient illumination comprises extracting a statistical intensity feature from a region of interest determined based at least in part on one or more of pupil segmentation or a defined region of the video frame, including an entire video frame, and at least one camera setting, wherein camera settings comprises one or more of an exposure time, a sensor gain, ISO, and white balance, and wherein the statistical intensity feature comprises one or more of a mean, a median, and a root-mean-square intensity value.

Clause 7. The pupillometry system of clause 5, wherein the computational model comprises a machine learning model trained using calibration data collected across multiple illumination levels wherein calibration data encodes a relationship between an ambient illumination level and statistical intensity features of the video frame.

Clause 8. The pupillometry system of clause 5, wherein estimating the level of ambient illumination comprises one or more of: compensating for stimulus illumination using one or more pre-stimulus frames, and excluding one or more frames affected by the light stimulus, and using one or more frames spanning a flash-onset transition or a flash-offset transition to compute a difference between (a) a first statistical intensity feature extracted from at least one frame immediately before the transition and (b) a second statistical intensity feature extracted from at least one frame immediately after the transition, and estimating the level of ambient illumination based at least in part on the difference.

Clause 9. The pupillometry system of clause 1, wherein computing the normalized pupil reactivity score comprises applying a function that maps a constriction metric to a normalized score using one or more parameters, wherein the one or more parameters are selected or calculated based on the determined ambient illumination.

Clause 10. The pupillometry system of clause 9, wherein the function comprises one or both an exponential saturation function and a sigmoidal function.

Clause 11. The pupillometry system of clause 1, further comprising classifying the eye as reactive or unreactive based on at least one threshold, wherein classifying the eye as unreactive comprises comparing a constriction amplitude or a change in pupil diameter to a threshold that depends on at least one of ambient illumination or baseline pupil diameter.

Clause 12. The pupillometry system of clause 1, wherein executing the instructions further causes the at least one processor to: determine one or more eye movement metrics based on the sequence of video frames using computer vision techniques comprising machine-learning-based detection or segmentation models, and determine a neurological status indicator based on the one or more eye movement metrics, wherein the one or more eye movement metrics include one or more of gaze deviation, fixation stability, saccade metrics, and nystagmus metrics.

Clause 13. The pupillometry system of clause 1, wherein executing the instructions further causes the at least one processor to use the light source to emit a baseline illumination during at least a portion of a pre-stimulus interval, the baseline illumination having an intensity at the eye less than about 50 lux.

Clause 14. The pupillometry system of clause 1, wherein executing the instructions further causes the at least one processor to determine one or both an intracranial hypertension risk classification and a predicted intracranial pressure value, based at least in part on the normalized pupil reactivity score.

Clause 15. The pupillometry system of clause 14, wherein executing the instructions further causes the at least one processor to generate an alert in response to determining that the intracranial hypertension risk classification exceeds a specified threshold.

Clause 16. The pupillometry system of clause 13, wherein intracranial hypertension corresponds to an intracranial pressure meeting or exceeding 20 mmHg.

Clause 17. The pupillometry system of clause 1, wherein executing the instructions further causes the at least one processor to generate a patient severity classification corresponding to a Glasgow Coma Scale category based at least in part on the normalized pupil reactivity score.

Clause 18. The pupillometry system of clause 1, wherein executing the instructions further causes the at least one processor to compute a quality score for the sequence of video frames, inhibit transmitting the normalized pupil reactivity score when the quality score fails to satisfy a threshold, and provide real-time guidance to a user to recapture the sequence.

Clause 19. The pupillometry system of clause 18, wherein the at least one processor computes the quality score based on one or more of a detected blink event, a measured gaze stability, magnitude of detected motion of the eye, focus of a video frame, presence of an occlusion within a video frame, and a determined stability of ambient illumination.

Clause 20. A computer-implemented method for pupillometry performed by a pupillometry system comprising an imaging device, a light source, a user interface, and at least one processor, the method comprising, by the at least one processor: capturing, using the imaging device, a sequence of video frames of an eye of a subject at least during a stimulus period when the eye is illuminated by a light stimulus; emitting, using the light source, the light stimulus during capture of the sequence of video frames; determining a level of ambient illumination associated with the sequence of video frames using one or more of a sensor signal, at least one video frame in the sequence video frames, and a camera exposure setting; determining one or more pupillary response parameters from the sequence of video frames; computing, a normalized pupil reactivity score based on the level of ambient illumination and the one or more pupillary response parameters; and transmitting the normalized pupil reactivity score to one or both the user interface and an external system separated from the pupillometry system.

Clause 21. A networked pupillometry system, comprising: a server system; and a plurality of pupillometry devices communicatively coupled to the server system, the plurality of pupillometry devices including at least a first pupillometry device and a second pupillometry device, wherein the first pupillometry device comprises: an imaging device configured to capture a sequence of video frames of an eye of a subject at least during a light stimulus period; a light source configured to emit a light stimulus toward the eye; and at least one processor and a memory storing instructions that, when executed by the at least one processor, cause the at least one processor to: generate, using the sequence of video frames, a first pupillometry measurement record comprising one or more pupillary response parameters for the eye of the subject; and transmit the first pupillometry measurement record to the server system, in association with a first patient identifier identifying the subject; wherein the server system comprises at least one server processor and a server memory storing instructions that, when executed, cause the server system to: receive a plurality of pupillometry measurement records from the plurality of pupillometry devices; store the plurality of pupillometry measurement records each in association with a patient identifier; and provide, to the second pupillometry device, a synchronized patient record view comprising at least the first pupillometry measurement record generated by the first pupillometry device for the patient identifier.

Clause 22. The system of clause 21, wherein the first pupillometry device is configured to, in response to loss or degradation of network connectivity, store the first pupillometry measurement record in a local data storage facility, queue the first pupillometry measurement record for later upload, and automatically synchronize the queued first pupillometry measurement record to the server system when connectivity is restored.

Clause 23. The system of clause 21, wherein the server system is configured to resolve a conflicting update to patient-associated data using a data conflict resolution process comprising one or more of timestamp-based precedence, field-level merging, and semantic analysis, and wherein the server system is configured to generate conflict-resolution logs comprising original values, resolved values, timestamps, and user identifications to form an audit trail.

Clause 24. The system of clause 21, wherein the server system is configured to enforce role-based access control for the synchronized patient record view and to apply cryptographic protection to the plurality of pupillometry measurement records during storage and network transmission.

Clause 25. The system of clause 21, wherein the server system comprises an electronic medical record (EMR) integration processor configured to transmit at least a portion of the first pupillometry measurement record to an EMR system using a standardized healthcare data exchange protocol comprising HL7 Fast Healthcare Interoperability Resources (FHIR).

Clause 26. The system of clause 21, wherein an individual pupillometry measurement record of the plurality of pupillometry measurement records is stored with one or both a model identifier and a version number associated with at least one computational model used by the system, and wherein the server system is configured to distribute one or both an updated model and an updated model parameter to at least a subset of the plurality of pupillometry devices.

Clause 27. The system of clause 26, wherein generating the first pupillometry measurement record further comprises performing biometric identification or authentication by combining one or more of an iris feature, a pupil dynamic, and an eye-movement feature, and performing liveness verification based on one or more of a dynamic pupillary response and an eye-movement signature.

Clause 28. The system of clause 21, wherein generating the first pupillometry measurement record further comprises determining ambient illumination and computing a normalized pupil reactivity score based on the ambient illumination and the one or more pupillary response parameters.

Clause 29. The system of clause 28, wherein generating the first pupillometry measurement record further comprises computing a quality score and implementing a two-tier quality process including real-time guidance and hard gating.

Clause 30. The system of clause 28, wherein generating the first pupillometry measurement record comprises multi-frame processing including frame alignment and multi-frame super-resolution.

Clause 31. The system of clause 28, wherein generating the first pupillometry measurement record further comprises generating a confounder-corrected output.

Clause 32. A computer-implemented method, comprising by a processor of a server system of a networked pupillometry system. receiving, from a plurality of pupillometry devices, measurement pupillometry records including a first measurement pupillometry record, in association with a first patient identifier, from a first pupillometry device of the plurality of pupillometry devices, wherein the first patient identifier identifies a subject; store at least the first pupillometry measurement record and the first patient identifier; and provide, to a second pupillometry device of the plurality of pupillometry devices, a synchronized patient record view comprising the first pupillometry measurement record generated by the first pupillometry device; wherein the first pupillometry comprises: an imaging device configured to capture a sequence of video frames of an eye of the subject at least during a light stimulus period; a light source configured to emit the light stimulus toward the eye; and at least one processor and a memory storing instructions that, when executed by the at least one processor, cause the at least one processor to: generate, using the sequence of video frames, a pupillometry measurement record comprising one or more pupillary response parameters for the eye of the subject; and transmit the pupillometry measurement record to the server system, in association with the first patient identifier.

Clause 33. The method of clause 32, further comprising offline queuing and later synchronization of pupillometry measurement records.

Clause 34. The method of clause 32, further comprising conflict resolution and audit logging.

Clause 35. The method of clause 32, further comprising transmitting pupillometry measurement records to an electronic medical record system.

Clause 36. The method of clause 32, further comprising storing a model identifier and/or version number with each pupillometry measurement record.

Clause 37. The method of clause 32, further comprising performing biometric identification and liveness verification using iris features, pupil dynamics, and eye-movement features.

Clause 38. The method of clause 32, further comprising computing a normalized pupil reactivity score based on ambient illumination.

Clause 39. The method of clause 32, further comprising computing a neurological status indicator based on pupillometry outputs.

Clause 40. A non-transitory computer-readable medium storing instructions that, when executed, by one or more processors, cause the one or more processors to perform operations comprising: receiving, from a plurality of pupillometry devices, measurement pupillometry records including a first measurement pupillometry record, in association with a first patient identifier, from a first pupillometry device of the plurality of pupillometry devices, wherein the first patient identifier identifies a subject; store at least the first pupillometry measurement record and the first patient identifier; and provide, to a second pupillometry device of the plurality of pupillometry devices, a synchronized patient record view comprising the first pupillometry measurement record generated by the first pupillometry device; wherein the first pupillometry comprises: an imaging device configured to capture a sequence of video frames of an eye of the subject at least during a light stimulus period; a light source configured to emit the light stimulus toward the eye; and at least one processor and a memory storing instructions that, when executed by the at least one processor, cause the at least one processor to: generate, using the sequence of video frames, a pupillometry measurement record comprising one or more pupillary response parameters for the eye of the subject; and transmit the pupillometry measurement record to the one or more processors, in association with the first patient identifier.

Clause 41. A non-transitory computer-readable medium storing instructions that, when executed, cause performance of the method of any one of clauses 32-39

Clause 42. A networked pupillometry system, comprising: a server system; and a plurality of pupillometry devices communicatively coupled to the server system, the plurality of pupillometry devices including at least a first pupillometry device having a first device configuration identifier and a second pupillometry device having a second device configuration identifier different from the first device configuration identifier, wherein each pupillometry device comprises: an imaging device configured to capture a sequence of video frames of an eye of a subject at least during a light stimulus period; a light source configured to emit a light stimulus toward the eye; and at least one processor and a memory storing instructions that, when executed by the at least one processor, cause the at least one processor to: determine the device configuration identifier for the pupillometry device; obtain, from the server system, a device-specific calibration parameter set associated with the device configuration identifier; estimate, using at least one video frame in the sequence and one or more camera settings and in accordance with the device-specific calibration parameter set, an ambient illumination level associated with the sequence; determine one or more pupillary response parameters for the eye of the subject from the sequence; compute a normalized pupil reactivity score based on the ambient illumination level and the one or more pupillary response parameters; generate a pupillometry measurement record comprising at least the normalized pupil reactivity score, the one or more pupillary response parameters, the device configuration identifier, and a version identifier for the device-specific calibration parameter set; and transmit the pupillometry measurement record to the server system in association with a patient identifier identifying the subject, wherein the server system comprises at least one server processor and a server memory storing instructions that, when executed, cause the server system to: store a plurality of device-specific calibration parameter sets each in association with a respective device configuration identifier, the plurality of device-specific calibration parameter sets comprising at least (i) a first device-specific calibration parameter set associated with the first device configuration identifier and (ii) a second device-specific calibration parameter set associated with the second device configuration identifier; receive a plurality of pupillometry measurement records from the plurality of pupillometry devices; store the plurality of pupillometry measurement records each in association with a patient identifier; and provide, to the second pupillometry device, a synchronized patient record view comprising at least a pupillometry measurement record associated with the patient identifier and generated by the first pupillometry device.

Clause 43. The system of clause 42, wherein the first pupillometry device is configured to, in response to loss or degradation of network connectivity, store a pupillometry measurement record in a local data storage facility, queue the pupillometry measurement record for later upload, and automatically synchronize the queued pupillometry measurement record to the server system when connectivity is restored.

Clause 44. The system of clause 42, wherein the server system is configured to resolve a conflicting update to patient-associated data using a data conflict resolution process comprising one or more of timestamp-based precedence, field-level merging, and semantic analysis, and wherein the server system is configured to generate conflict-resolution logs comprising original values, resolved values, timestamps, and user identifications to form an audit trail.

Clause 45. The system of clause 42, wherein the server system is configured to enforce role-based access control for the synchronized patient record view and to apply cryptographic protection to the plurality of pupillometry measurement records during storage and network transmission.

Clause 46. The system of clause 42, wherein the server system comprises an electronic medical record (EMR) integration processor configured to transmit at least a portion of a pupillometry measurement record to an EMR system using a standardized healthcare data exchange protocol comprising HL7 Fast Healthcare Interoperability Resources (FHIR).

Clause 47. The system of clause 42, wherein an individual pupillometry measurement record of the plurality of pupillometry measurement records is stored with one or both a model identifier and a version number associated with at least one computational model used to estimate the ambient illumination level or compute the normalized pupil reactivity score, and wherein the server system is configured to distribute one or both an updated model and an updated model parameter set to at least a subset of the plurality of pupillometry devices.

Clause 48. The system of clause 42, wherein generating a pupillometry measurement record further comprises performing biometric identification or authentication by combining one or more of an iris feature, a pupil dynamic, and an eye-movement feature, and performing liveness verification based on one or more of a dynamic pupillary response and an eye-movement signature.

Clause 49. The system of clause 42, wherein the device-specific calibration parameter set comprises one or more device-specific coefficients that relate one or more statistical intensity features derived from at least one video frame in the sequence and one or more of the camera settings to the ambient illumination level, and wherein the camera settings comprise one or more of exposure time, sensor gain, ISO, and white balance.

Clause 50. The system of clause 42, wherein computing the normalized pupil reactivity score comprises applying a saturation function that maps a raw pupillary response metric to the normalized pupil reactivity score, wherein one or more parameters of the saturation function are set based on the ambient illumination level.

Clause 51. The system of clause 1, wherein at least the first pupillometry device is configured to adjust an intensity of the light stimulus emitted by the light source based on (i) an estimated device-to-eye distance and (ii) the ambient illumination level.

Clause 52. The system of clause 47, wherein distributing one or both the updated model and the updated model parameter set comprises federated learning in which local model updates are computed on-device and aggregated by the server system without transmitting raw video frames from the plurality of pupillometry devices.

Clause 53. A computer-implemented method performed by a processor of a server system of a networked pupillometry system, the method comprising: storing, in the server system, a plurality of device-specific calibration parameter sets each in association with a respective device configuration identifier; receiving, from a first pupillometry device of a plurality of pupillometry devices, a request including a first device configuration identifier; in response to the request, selecting a first device-specific calibration parameter set associated with the first device configuration identifier, and providing the first device-specific calibration parameter set to the first pupillometry device; receiving, from the first pupillometry device and in association with a patient identifier identifying a subject, a first pupillometry measurement record comprising: (i) a normalized pupil reactivity score computed based on an ambient illumination level estimated in accordance with the first device-specific calibration parameter set and (ii) (ii) one or more pupillary response parameters, and further comprising a version identifier for the first device-specific calibration parameter set; storing the first pupillometry measurement record in association with the patient identifier; and providing, to a second pupillometry device of the plurality of pupillometry devices, a synchronized patient record view comprising the first pupillometry measurement record.

Clause 54. The method of clause 53, wherein receiving the first pupillometry measurement record comprises receiving the first pupillometry measurement record after the first pupillometry device queues the first pupillometry measurement record while operating in an offline mode and thereafter restores network connectivity.

Clause 55. The method of clause 53, further comprising resolving, by the server system, a conflicting update to patient-associated data using one or more of timestamp-based precedence, field-level merging, and semantic analysis, and generating a conflict-resolution log comprising timestamps and user identifications to form an audit trail.

Clause 56. The method of clause 53, further comprising enforcing, by the server system, role-based access control for the synchronized patient record view and applying cryptographic protection to pupillometry measurement records during storage and network transmission.

Clause 57. The method of clause 53, further comprising transmitting, by the server system, at least a portion of the first pupillometry measurement record to an electronic medical record system using an HL7 FHIR protocol handler.

Clause 58. The method of clause 53, further comprising storing a model identifier and/or version number with the first pupillometry measurement record, and distributing an updated model and/or an updated model parameter set to at least a subset of the plurality of pupillometry devices.

Clause 59. The method of clause 58, wherein distributing the updated model and/or the updated model parameter set comprises federated learning in which local updates are computed on-device and aggregated without transmitting raw video frames from the plurality of pupillometry devices.

Clause 60. The method of clause 53, wherein the first pupillometry measurement record further comprises the first device configuration identifier and one or more camera settings used to estimate the ambient illumination level.

Clause 61. A non-transitory computer-readable medium storing instructions that, when executed by one or more processors of a server system of a networked pupillometry system, cause the one or more processors to perform the method of any one of clauses 53-60.

### Terminology

Further, each operation can include any combination of techniques implemented by the embodiments described herein.

Using the teachings disclosed herein, a skilled artisan can implement the above-described systems and methods in digital electronic circuitry, computer hardware, firmware, or software. The implementation can be a computer program product. Additionally, the implementation can include a machine-readable storage device for execution by or to control the operation of a data processing apparatus. The implementation can, for example, be a programmable processor, a computer, or multiple computers.

A computer program can be in any programming language, including compiled or interpreted languages. The computer program can have any deployed form, including a stand-alone program, subroutine, element, or other units suitable for a computing environment. One or more computers can execute a deployed computer program.

One or more programmable processors can perform the method steps by executing a computer program to perform the concepts described herein by operating on input data and generating output. An apparatus can also perform the steps of the method. The apparatus can be a special-purpose logic circuitry. For example, the circuitry is an FPGA (field-programmable gate array) or an ASIC (application-specific integrated circuit). Subroutines and software agents can refer to portions of the computer program, the processor, the special circuitry, software, or hardware that implements that functionality.

Processors suitable for executing a computer program include, by way of example, both general and special purpose microprocessors and any one or more processors of any digital computer. A processor can receive instructions and data from a read-only memory, a random-access memory, or both. Thus, for example, a computer's essential elements are a processor for executing instructions and one or more memory devices for storing instructions and data. Additionally, a computer can receive data from or transfer data to one or more mass storage device(s) for storing data (e.g., magnetic, magneto-optical disks, solid-state drives (SSDs), hard disk drives, and flash memory).

Data transmission and instructions can also occur over a communications network. Information carriers that embody computer program instructions and data include all nonvolatile memory forms, including semiconductor memory devices. The information carriers can, for example, be EPROM, EEPROM, flash memory devices, magnetic disks, internal hard disks, removable disks, solid-state drives, and flash memory devices. In addition, the processor and the memory can be supplemented by or incorporated into special-purpose logic circuitry.

A computer with a display device enabling user interaction can implement the above-described techniques, such as a display, keyboard, mouse, or any other input/output peripheral. The display device can, for example, be a cathode ray tube (CRT) or a liquid crystal display (LCD) monitor. The user can provide input to the computer (e.g., interact with a user interface element). In addition, other kinds of devices can enable user interaction. Other devices can, for example, be feedback provided to the user in any form of sensory feedback (e.g., visual feedback, auditory feedback, or tactile feedback). For example, input from the user can be in any form, including acoustic, speech, or tactile input.

A distributed computing system with a back-end component can also implement the above-described techniques. The back-end component can, for example, be a data server, a middleware component, or an application server. Further, a distributing computing system with a front-end component can implement the above-described techniques. The front-end component can, for example, be a client computer with a graphical user interface, a web browser through which a user can interact with an example implementation or other graphical user interfaces for a transmitting device. Finally, the system's components can interconnect using any form or medium of digital data communication (e.g., a communication network). Examples of communication network(s) include a local area network (LAN), a wide area network (WAN), the Internet, a wired network(s), or a wireless network(s).

The system can include a client(s) and server(s). The client and server (e.g., a remote server) can interact through a communication network. For example, a client-and-server relationship can arise when computer programs run on the respective computers and have a client-server relationship. Further, the system can include a storage array(s) that delivers distributed storage services to the client(s) or server(s).

Packet-based network(s) can include, for example, the Internet, a carrier internet protocol (IP) network (e.g., local area network (LAN), wide area network (WAN), campus area network (CAN), metropolitan area network (MAN), home area network (HAN)), a private IP network, an IP private branch exchange (IPBX), a wireless network (e.g., radio access network (RAN), IEEE 802.11 network(s), IEEE 802.16 network(s), general packet radio service (GPRS) network, HiperLAN), or other packet-based networks. Circuit-based network(s) can include, for example, a public switched telephone network (PSTN), a private branch exchange (PBX), a wireless network, or other circuit-based networks. Finally, wireless network(s) can include RAN, Bluetooth, code-division multiple access (CDMA) networks, time division multiple access (TDMA) networks, and global systems for mobile communications (GSM) networks.

The transmitting device can include, for example, a computer, a computer with a browser device, a telephone, an IP phone, a mobile device (e.g., cellular phone, personal digital assistant (PDA) device, laptop computer, electronic mail device), or other communication devices. The browser device includes, for example, a computer (e.g., desktop computer, laptop computer) with a World Wide Web browser (e.g., Safari® or Chrome®). The mobile computing device includes, for example, a smartphone.

Comprise, include, or plural forms of each are open-ended, include the listed parts, and contain additional unlisted elements. Unless explicitly disclaimed, the term ‘or’ is open-ended and includes one or more of the listed parts, items, elements, and combinations thereof.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Disjunctive language such as the phrase “at least one of X, Y, or Z,” unless specifically stated otherwise, is otherwise understood with the context as used in general to present that an item, term, etc., may be either X, Y, or Z, or any combination thereof (for example, X, Y, and/or Z). Thus, such disjunctive language is not generally intended to, and should not, imply that certain examples require at least one of X, at least one of Y, or at least one of Z to each be present.

All figures, tables, and appendices, as well as patents, applications, and publications, referred to above, are hereby incorporated by reference. Additionally, all publications, patents, and patent applications mentioned in this specification are herein incorporated by reference to the same extent as if each individual publication, patent, or patent application was specifically and individually indicated to be incorporated by reference.

Some embodiments have been described in connection with the accompanying drawing. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

In this description, references to “an embodiment,” “one embodiment,” or the like, mean that the particular feature, function, structure or characteristic being described is included in at least one embodiment of the technique introduced herein. Occurrences of such phrases in this specification do not necessarily all refer to the same embodiment. On the other hand, the embodiments referred to are also not necessarily mutually exclusive

What is claimed is:

1. A networked pupillometry system, comprising:
a server system; and a plurality of pupillometry devices communicatively coupled to the server system, the plurality of pupillometry devices including at least a first pupillometry device having a first device configuration identifier and a second pupillometry device having a second device configuration identifier different from the first device configuration identifier, wherein each pupillometry device comprises:
an imaging device configured to capture a sequence of video frames of an eye of a subject at least during a light stimulus period; a light source configured to emit a light stimulus toward the eye; and at least one processor and a memory storing instructions that, when executed by the at least one processor, cause the at least one processor to:
determine the device configuration identifier for the pupillometry device;
obtain, from the server system, a device-specific calibration parameter set associated with the device configuration identifier;
estimate, using at least one video frame in the sequence and one or more camera settings and in accordance with the device-specific calibration parameter set, an ambient illumination level associated with the sequence;
determine one or more pupillary response parameters for the eye of the subject from the sequence; compute a normalized pupil reactivity score based on the ambient illumination level and the one or more pupillary response parameters;
generate a pupillometry measurement record comprising at least the normalized pupil reactivity score, the one or more pupillary response parameters, the device configuration identifier, and a version identifier for the device-specific calibration parameter set; and
transmit the pupillometry measurement record to the server system in association with a patient identifier identifying the subject,
wherein the server system comprises at least one server processor and a server memory storing instructions that, when executed, cause the server system to:
store a plurality of device-specific calibration parameter sets each in association with a respective device configuration identifier, the plurality of device-specific calibration parameter sets comprising at least (i) a first device-specific calibration parameter set associated with the first device configuration identifier and (ii) a second device-specific calibration parameter set associated with the second device configuration identifier;
receive a plurality of pupillometry measurement records from the plurality of pupillometry devices;
store the plurality of pupillometry measurement records each in association with a patient identifier; and
provide, to the second pupillometry device, a synchronized patient record view comprising at least a pupillometry measurement record associated with the patient identifier and generated by the first pupillometry device.

2. The system of claim 1, wherein the first pupillometry device is configured to, in response to loss or degradation of network connectivity, store a pupillometry measurement record in a local data storage facility, queue the pupillometry measurement record for later upload, and automatically synchronize the queued pupillometry measurement record to the server system when connectivity is restored.

3. The system of claim 1, wherein the server system is configured to resolve a conflicting update to patient-associated data using a data conflict resolution process comprising one or more of timestamp-based precedence, field-level merging, and semantic analysis, and wherein the server system is configured to generate conflict-resolution logs comprising original values, resolved values, timestamps, and user identifications to form an audit trail.

4. The system of claim 1, wherein the server system is configured to enforce role-based access control for the synchronized patient record view and to apply cryptographic protection to the plurality of pupillometry measurement records during storage and network transmission.

5. The system of claim 1, wherein the server system comprises an electronic medical record (EMR) integration processor configured to transmit at least a portion of a pupillometry measurement record to an EMR system using a standardized healthcare data exchange protocol comprising HL7 Fast Healthcare Interoperability Resources (FHIR).

6. The system of claim 1, wherein an individual pupillometry measurement record of the plurality of pupillometry measurement records is stored with one or both a model identifier and a version number associated with at least one computational model used to estimate the ambient illumination level or compute the normalized pupil reactivity score, and wherein the server system is configured to distribute one or both an updated model and an updated model parameter set to at least a subset of the plurality of pupillometry devices.

7. The system of claim 6, wherein distributing one or both the updated model and the updated model parameter set comprises federated learning in which local model updates are computed on-device and aggregated by the server system without transmitting raw video frames from the plurality of pupillometry devices.

8. The system of claim 1, wherein generating a pupillometry measurement record further comprises performing biometric identification or authentication by combining one or more of an iris feature, a pupil dynamic, and an eye-movement feature, and performing liveness verification based on one or more of a dynamic pupillary response and an eye-movement signature.

9. The system of claim 1, wherein the device-specific calibration parameter set comprises one or more device-specific coefficients that relate one or more statistical intensity features derived from at least one video frame in the sequence and one or more of the camera settings to the ambient illumination level, and wherein the camera settings comprise one or more of exposure time, sensor gain, ISO, and white balance.

10. The system of claim 1, wherein computing the normalized pupil reactivity score comprises applying a saturation function that maps a raw pupillary response metric to the normalized pupil reactivity score, wherein one or more parameters of the saturation function are set based on the ambient illumination level.

11. The system of claim 1, wherein at least the first pupillometry device is configured to adjust an intensity of the light stimulus emitted by the light source based on (i) an estimated device-to-eye distance and (ii) the ambient illumination level.

12. A computer-implemented method performed by a processor of a server system of a networked pupillometry system, the method comprising:
storing, in the server system, a plurality of device-specific calibration parameter sets each in association with a respective device configuration identifier;
receiving, from a first pupillometry device of a plurality of pupillometry devices, a request including a first device configuration identifier; in response to the request,
selecting a first device-specific calibration parameter set associated with the first device configuration identifier, and
providing the first device-specific calibration parameter set to the first pupillometry device;
receiving, from the first pupillometry device and in association with a patient identifier identifying a subject, a first pupillometry measurement record comprising:
(i) a normalized pupil reactivity score computed based on an ambient illumination level estimated in accordance with the first device-specific calibration parameter set and
(ii) (ii) one or more pupillary response parameters, and further comprising a version identifier for the first device-specific calibration parameter set;
storing the first pupillometry measurement record in association with the patient identifier; and
providing, to a second pupillometry device of the plurality of pupillometry devices, a synchronized patient record view comprising the first pupillometry measurement record.

13. The method of claim 12, wherein receiving the first pupillometry measurement record comprises receiving the first pupillometry measurement record after the first pupillometry device queues the first pupillometry measurement record while operating in an offline mode and thereafter restores network connectivity.

14. The method of claim 12, further comprising resolving, by the server system, a conflicting update to patient-associated data using one or more of timestamp-based precedence, field-level merging, and semantic analysis, and generating a conflict-resolution log comprising timestamps and user identifications to form an audit trail.

15. The method of claim 12, further comprising enforcing, by the server system, role-based access control for the synchronized patient record view and applying cryptographic protection to pupillometry measurement records during storage and network transmission.

16. The method of claim 12, further comprising transmitting, by the server system, at least a portion of the first pupillometry measurement record to an electronic medical record system using an HL7 FHIR protocol handler.

17. The method of claim 12, further comprising storing a model identifier and/or version number with the first pupillometry measurement record, and distributing an updated model and/or an updated model parameter set to at least a subset of the plurality of pupillometry devices.

18. The method of claim 17, wherein distributing the updated model and/or the updated model parameter set comprises federated learning in which local updates are computed on-device and aggregated without transmitting raw video frames from the plurality of pupillometry devices.

19. The method of claim 12, wherein the first pupillometry measurement record further comprises the first device configuration identifier and one or more camera settings used to estimate the ambient illumination level.

20. A non-transitory computer-readable medium storing instructions that, when executed by one or more processors of a server system of a networked pupillometry system, cause the one or more processors to perform the method of claim 12.
